(19) United States
(12) Patent Application Publication
(10) Pub. No.: US 2008/0027044 A1
(43) Pub. Date: Jan. 31, 2008

(54) Prodrug antibiotic screens

(76) Inventors: Kim Lewis, Newton, MA (US); Gabriele Casadei, Cambridge, MA (US)

Correspondence Address: WILMERHALE/BOSTON, 60 STATE STREET, BOSTON, MA, 02109, US

(21) Appl. No.: 11/818,166

(22) Filed: Jun. 13, 2007

Related U.S. Application Data
(60) Provisional application No. 60/813,171, filed on Jun. 13, 2006.

(51) Int. Cl. A61K 31/549; A61K 31/17; A61K 31/351; A61K 31/381; A61K 31/40; A61K 31/403; A61P 43/00; A61P 31/00; A61K 31/4184; A61K 31/435; A61K 31/498; A61K 31/4985

(52) U.S. Cl. 514/223.8; 514/235.2; 514/256; 514/266.4; 514/267; 514/290; 514/312; 514/314; 514/394; 514/395; 514/411; 514/422; 514/443; 514/445; 514/448; 514/459; 514/585; 514/596

(57) ABSTRACT

Methods for identifying prodrug activity utilizing screens with microbial mutants, including null and conditional mutants, transiently gene-repressed microbes, and gene-overexpressing microbes, as well as pooled collections of these are provided. Effective prodrug antibiotics differentially kill or inhibit microbes overexpressing prodrug activating genes and allow non-expressing strains to grow and be readily identified. Methods for detecting prodrug antibiotic activity by detecting a lack of differential sensitivity of multidrug efflux mutant or deficient strains are also provided. Novel pharmaceutical compounds identified by the methods of the invention, pharmaceutical formulations, and methods for treating an infection comprising administering to a subject in need thereof an effective amount of a Compound of the Invention are also provided.

### CROSS REFERENCE TO RELATED APPLICATIONS

This application claims priority of U.S. Provisional Appln. No. 60/813,171, filed Jun. 13, 2006, which is incorporated herein in its entirety.

### FIELD OF THE INVENTION

The invention is in the field of microbial chemistry.

### BACKGROUND OF THE INVENTION

A renewed focus on antimicrobial drug discovery is critical as pathogens are getting increasingly more resistant to available drugs (Lewis et al. (2002) Drug Efflux. In “Bacterial Resistance to Antimicrobials: Mechanisms, Genetics, Medical Practice and Public Health.” Lewis, K., Salyers A., Taber H. and Wax, R. (eds). New York: Marcel Dekker, pp. 61-90; Levy et al. (2004) Nat. Med. 10:S122-129). If novel compounds are not developed, there would be a return to the pre-antibiotic era of epidemics and pandemics. A full-scale epidemic caused by an untreatable infection could change the way of present life. In this regard, infectious disease may be the single most important unsolved human health problem.

Virtually all classes of antibiotics in use today derive from natural product discoveries made in the 1940's and 1950's (Walsh, C. (2003a) Antibiotics. Actions, Origins, Resistance. Washington, DC: ASM Press). Culturable microorganisms, the source of most antibiotics, make up only approximately 1% of the total number of microbial species and their over-mining largely accounts for the end of the “golden era” of antibiotic discovery (Osbourne et al. (2000) ASM News 66:411-417). Synthetic approaches produced a single class of compounds, the fluoroquinolones based on the natural product, nalidixic acid. The fluoroquinolones are the only broad-spectrum synthetic class, and the only new addition to broad-spectrum compounds in the last 40 years. The next novel class of synthetics, the oxazolidinones, was discovered 30 years later, and only acts against Gram-positive species of bacteria.

A major need in antimicrobial drug discovery is for novel broad-spectrum compounds, because in many, if not most, cases there is simply not time to identify the exact nature of the pathogen. This is especially true in the case of a bioterrorist attack.

Synthetic compounds have thus far failed to replace natural antibiotics and lead to novel classes of broad-spectrum compounds, despite the combined efforts of combinatorial synthesis, high-throughput screening, advanced medicinal chemistry, genomics and proteomics, and rational drug design. As a result, many companies have closed their anti-infective divisions (see, e.g., Dougherty et al. (2002) Curr. Pharm. Des. 8:1119-1135; Boggs et al. (2004) Clin. Microbiol. Infect. 10 Suppl 4:32-36; Bush, K. (2004) Clin. Microbiol. Infect. 10 Suppl 4:10-17; Projan et al. (2004) Clin. Microbiol. Infect. 10 Suppl 4:18-22; Schmid, M. B. (2004) Nat. Rev. Microbiol. 2:739-746; Silver, L. (2005) IDrugs 8(8):651-655).

The problem with obtaining new synthetic antibiotics may be related in part to the fact that the synthetic antibiotics are invariably pumped out across the outer membrane barrier of Gram-negative bacteria by Multidrug Resistance pumps (MDRs). In the early 1990s, the first “trans-envelope” MDR in E. coli that can extrude chemically unrelated compounds from the cell was described (Lomovskaya et al. (1992) Proc. Natl. Acad. Sci. USA 89:8938-8942). Many additional broad-spectrum MDRs were subsequently discovered (Ma et al. (1993) J. Bacteriol. 175:6299-6313; Poole et al. (1993) J. Bacteriol. 175:7363-7372; Hagman et al. (1995) Microbiol. 141:611-622). The outer membrane of Gram-negative bacteria is a barrier for amphipathic compounds (which essentially all drugs are), and MDRs extrude drugs across this barrier. Evolution produced antibiotics that can largely bypass this dual barrier/extrusion mechanism, but synthetic compounds almost invariably fail. There is not currently available a rational means to create compounds that will be both active and capable of penetrating into Gram-negative bacteria. Blocking the MDRs is a possibility (Stermitz et al. (2002) Planta. Med. 68:1140-1141) (Renau et al. (2002) Bioorg. Med. Chem. Lett. 12:763-766), but whether it is realistic to discover a non-toxic, “broad-spectrum” MDR inhibitor remains unclear.

Apart from broad-spectrum compounds, there is an even greater unmet need for sterilizing antimicrobials. The inability to sterilize an infection is accepted as an inevitable shortcoming of antibiotics, and approaches to develop such compounds are simply non-existent (Lewis, K. (2001a) Chemother. 45:999-1007; Coates et al. (2002) Nat. Rev. Drug Discov. 1:895-910). The FDA only requires testing of rapidly propagating cultures for drug approval. Yet, the unmet need for compounds that can eradicate, rather than suppress, an infection and be effective against slow-growing biofilm infections is acute.

Methods of identifying broad-spectrum or sterilizing compounds are currently non-existent. This application fulfills these unmet needs.

### SUMMARY OF THE INVENTION

Aspects of the invention are based, in part, upon a new strategy for screening compounds for prodrug antibiotic activity by examining mutant populations of microorganisms or populations of microorganisms with transiently suppressed prodrug-activating gene expression, for their relative resistance to such active prodrug compounds when compared to an otherwise isogenic wild-type microorganism. The invention is also based in part upon the enhanced sensitivity to prodrug compounds of microbial strains in which prodrug-activating genes are overexpressed or their function is otherwise enhanced.

Accordingly, in one aspect, the invention provides a method of identifying a compound with prodrug antibiotic activity. The method includes contacting a mutant microbial organism that is mutant in one or more genes with a candidate prodrug antibiotic compound; detecting a level of growth of the mutant microbial organism in the presence of the candidate prodrug antibiotic compound; and comparing the level of growth of the mutant microbial organism in the presence of the candidate prodrug antibiotic compound to the level of growth of a wild-type microbial organism in the presence of the candidate prodrug antibiotic compound. The wild-type microbial organism is not mutant in the one or more genes that are mutant in the mutant microbial organism. By this method, a greater level of growth of the mutant microbial organism in the presence of the candidate compound than the level of growth of the wild-type microbial organism in the presence of the candidate compound is indicative of a prodrug antibiotic activity of the candidate compound.

In some embodiments, the microbial organism is a bacterium (e.g., a strain of Escherichia coli, Salmonella typhimurium, Staphylococcus aureus, Pseudomonas aeruginosa, Haemophilus influenza, Mycobacterium tuberculosis, Enterococcus faecalis, etc.). In other embodiments, the microbial organism is a fungal organism (e.g., a yeast such as Candida albicans, Candida glabrata, Saccharomyces cerevisiae, Aspergillus nidulans, Aspergillus, fumigatus, Aspergillus niger, etc.). In yet other embodiments, the microbial organism is a protozoan (e.g., Cryptosporidium parvum, Naegleria fowleri, Acanthomeaba SPP, Isospora belli, Microsporidia, etc.). In particular embodiments, the mutant microbial organism has a loss-of-function mutation in one or more genes, and, in some cases the loss-of-function mutation is a null mutation. In other embodiments, the mutant microbial organism has a conditional mutation in one or more genes, and in some cases, the conditional mutation is a temperature-sensitive mutation. In certain embodiments, the conditional mutation is in an essential gene.

In further embodiments, the prodrug antibiotic activity kills or inhibits the growth of a pathogenic organism. In some embodiments, the pathogenic organism is a bacterium. In particular embodiments, the bacterium belong to various Gram-positive and Gram-negative bacterial strains such as Bacillus, Burkholderia, Enterobacter, Escherichia, Helicobacter, Klebsiella, Mycobacterium, Neisseria, Pseudomonas, Staphylococcus, Streptococcus, and Yersinia including drug resistant strains thereof. Non-limiting examples of bacterial pathogenic organisms include Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, and Yersinia pestis. In still further embodiments, the prodrug antibiotic activity kills or inhibits the growth of a pathogenic fungal organism such as Candida albicans, Candida glabrata, Aspergillus niger, Aspergillus fumigatus, Cryptococcus neoformans, and Pneumocystis carinii. In other embodiments, the prodrug antibiotic activity kills or inhibits the growth of a pathogenic helminth (e.g., flatworms (flukes and tapeworms) and roundworms). In further embodiments, the prodrug antibiotic activity kills or inhibits the growth of a pathogenic protozoan organism such as Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleriafowleri, Acanthomeaba SPP, Isospora belli, and Microsporidia.

In particular embodiments, the level of microbial growth is determined in a liquid growth medium. In other embodiments, the level of microbial growth is determined in a plate assay. In certain embodiments, the step of contacting the mutant microbial organism with the candidate prodrug antibiotic compound comprises simultaneously contacting a plurality of mutant microbial organisms that are mutant in different genes. In particular embodiments, the plurality of mutant microbial organisms that are mutant in different genes is a mutant library of otherwise isogenic microbes having singular mutational identities. By this embodiment of the invention, the precise mutant prodrug activating gene affecting activation of a candidate prodrug compound need not be known in advance, and the combined mutatnt pool can be readily screened for resistant outgrowers corresponding to the mutants affecting this relevant prodrug activating gene.

In yet further particular embodiments, the candidate prodrug antibiotic compound is one found in a chemical library, such as the Compound Library of The New England Regional Center of Excellence for Biodefense and Emerging Infectious Diseases, the Compound Library of the National Institutes of Health Molecular Library Screening Center, The ChemBridge Library, the ChemDiv Library, and the MayBridge Library.

In another aspect, the invention provides a method of identifying a compound with prodrug antibiotic activity which includes the steps of contacting a microbial organism with a candidate prodrug antibiotic compound while one or more genes of the organism is repressed; detecting a level of growth of the gene-repressed microbial organism in the presence of the candidate prodrug antibiotic compound; and comparing the level of growth of the gene-repressed microbial organism in the presence of the candidate prodrug antibiotic compound to the level of growth of the same microbial organism in which the one or more genes of the organism is not repressed in the presence of the same candidate prodrug antibiotic compound. By this method, a greater level of growth of the gene-repressed microbial organism in the presence of the compound than the level of growth of the non-gene-repressed microbial organism in the presence of the compound is indicative of a prodrug antibiotic activity of the candidate compound.

In some embodiments, the one or more genes that are repressed in the gene-repressed microbial organism are repressed using a composition such as an antisense oligonucleotide, a ribozyme, a small interfering RNA, or an aptamer. In other embodiments, the gene's activity is regulated by a temperature sensitive mutation or by an inducible promoter or other regulatory element(s). In particular embodiments, the one or more genes that are repressed in the gene-repressed microbial organism are repressed using antisense to the one or more genes of the organism, e.g. an antisense RNA transcript that is produced from a partial or complete cDNA of the one or more genes cloned behind a promoter in the antisense orientation. In certain embodiments, the one or more of the genes being repressed is an essential gene.

In some embodiments, the microbial organism is a bacterium (e.g., a strain of Escherichia coli, Salmonella typhimurium, Staphylococcus aureus, Pseudomonas aeruginosa, Haemophilus influenza, Mycobacterium tuberculosis, Enterococcus faecalis, etc.). In other embodiments, the microbial organism is a fungal organism (e.g., a yeast such as Candida albicans, Candida glabrata, Saccharomyces cerevisiae, Aspergillus nidulans, Aspergillus, fumigatus, Aspergillus niger, etc.). In yet other embodiments, the microbial organism is a protozoan (e.g., Cryptosporidium parvum, Naegleria fowleri, Acanthomeaba SPP, Isospora belli, Microsporidia, etc.). In particular embodiments, the mutant microbial organism has a loss-of-function mutation in one or more genes, and, in some cases the loss-of-function mutation is a null mutation. In other embodiments, the mutant microbial organism has a conditional mutation in one or more genes, and in some cases, the conditional mutation is a temperature-sensitive mutation. In certain embodiments, the conditional mutation is in an essential gene.

In further embodiments, the prodrug antibiotic activity kills or inhibits the growth of a pathogenic organism. In some embodiments, the pathogenic organism is a bacterium. In particular embodiments, the bacterium belong to various Gram-positive and Gram-negative bacterial strain such as Bacillus, Burkholderia, Enterobacter, Escherichia, Helicobacter, Klebsiella, Mycobacterium, Neisseria, Pseudomonas, Staphylococcus, Streptococcus, and Yersinia including drug resistant strains thereof. Non-limiting examples of bacterial pathogenic organisms include Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacterpylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, and Yersinia pestis. In still further embodiments, the prodrug antibiotic activity kills or inhibits the growth of a pathogenic fungal organism such as Candida albicans, Candida glabrata, Aspergillus niger, Aspergillus fumigatus, Cryptococcus neoformans, and Pneumocystis carinii. In other embodiments, the prodrug antibiotic activity kills or inhibits the growth of a pathogenic helminth (e.g., flatworms (flukes and tapeworms) and roundworms). In further embodiments, the prodrug antibiotic activity kills or inhibits the growth of a pathogenic protozoan organism such as Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, and Microsporidia.

In particular embodiments, the level of microbial growth is determined in a liquid growth medium. In other embodiments, the level of microbial growth is determined in a plate assay. In yet further particular embodiments, the candidate prodrug antibiotic compound is one found in a chemical library such as the Compound Library of The New England Regional Center of Excellence for Biodefense and Emerging Infectious Diseases, the Compound Library of the National Institutes of Health Molecular Library Screening Center, The ChemBridge Library, the ChemDiv Library, or the MayBridge Library.

In certain embodiments, the step of contacting the gene-repressed microbial organism with the candidate prodrug antibiotic compound comprises simultaneously contacting a plurality of distinct gene-repressed microbial organisms that are repressed in distinct genes but otherwise isogenic. In particular embodiments, the plurality of distinct gene-repressed microbial organisms that are repressed in distinct genes but otherwise isogenic is a microbial organism that is transformed with a cDNA library expressing antisense cDNA derived from the same microbial organism. By this embodiment of the invention, the precise mutant prodrug activating gene affecting activation of a candidate prodrug compound need not be known in advance, and the combined gene-repressed microbial pool can be readily screened for resistant outgrowers corresponding to the genes affecting this relevant prodrug activating gene.

In yet another aspect, the invention provides a method of identifying a compound with prodrug antibiotic activity. In this method, a microbial organism is contacted with a candidate prodrug antibiotic compound while one or more genes of the organism is overexpressed. A level of growth of the overexpressing microbial organism is detected in the presence of the candidate prodrug antibiotic compound. Then, the level of growth of the overexpressing microbial organism in the presence of the candidate prodrug antibiotic compound is compared to the level of growth in the presence of the same candidate prodrug antibiotic compound of the same microbial organism which is not overexpressing the one or more genes. By this method, a lower level of growth of the overexpressing microbial organism in the presence of the candidate compound than the level of growth of the non-overexpressing microbial organism in the presence of the candidate compound is indicative of a prodrug antibiotic activity of the candidate compound.

In some embodiments, the overexpressed gene is a prodrug-activating gene. In certain embodiments, the one or more genes that are overexpressed in the overexpressing microbial organism are overexpressed using a cDNA of the one or more genes of the organism. In particular embodiments, the cDNA is overexpressed using a strong promoter that is active in the microbial organism. In further embodiments, one or more of the genes being overexpressed is an essential gene.

In some embodiments, the microbial organism is a bacterium (e.g., a strain of Escherichia coli, Salmonella typhimurium, Staphylococcus aureus, Pseudomonas aeruginosa, Haemophilus influenza, Mycobacterium tuberculosis, Enterococcus faecalis, etc.). In other embodiments, the microbial organism is a fungal organism (e.g., a yeast such as Candida albicans, Candida glabrata, Saccharomyces cerevisiae, Aspergillus nidulans, Aspergillus, fumigatus, Aspergillus niger, etc.). In yet other embodiments, the microbial organism is a protozoan (e.g., Cryptosporidium parvum, Naegleria fowleri, Acanthomeaba SPP, Isospora belli, Microsporidia, etc.). In particular embodiments, the mutant microbial organism has a loss-of-function mutation in one or more genes, and, in some cases the loss-of-function mutation is a null mutation. In other embodiments, the mutant microbial organism has a conditional mutation in one or more genes, and in some cases, the conditional mutation is a temperature-sensitive mutation. In certain embodiments, the conditional mutation is in an essential gene.

In further embodiments, the prodrug antibiotic activity kills or inhibits the growth of a pathogenic organism. In some embodiments, the pathogenic organism is a bacterium. In particular embodiments, the bacterium belong to various Gram-positive and Gram-negative bacterial strains such as Bacillus, Burkholderia, Enterobacter, Escherichia, Helicobacter, Klebsiella, Mycobacterium, Neisseria, Pseudomonas, Staphylococcus, Streptococcus, and Yersinia including drug resistant strains thereof. Non-limiting examples of bacterial pathogenic organisms include Escherichia coli, Escherichia coli O15 7:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, and Yersinia pestis. In still further embodiments, the prodrug antibiotic activity kills or inhibits the growth of a pathogenic fungal organism such as Candida albicans, Candida glabrata, Aspergillus niger, Aspergillus fumigatus, Cryptococcus neoformans, and Pneumocystis carinii. In other embodiments, the prodrug antibiotic activity kills or inhibits the growth of a pathogenic helminth (e.g., flatworms (flukes and tapeworms) and roundworms). In further embodiments, the prodrug antibiotic activity kills or inhibits the growth of a pathogenic protozoan organism such as Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, and Microsporidia.

In particular embodiments, the level of microbial growth is determined in a liquid growth medium. In other embodiments, the level of microbial growth is determined in a plate assay. In yet further particular embodiments, the candidate prodrug antibiotic compound may be one found in a chemical library such as the Compound Library of The New England Regional Center of Excellence for Biodefense and Emerging Infectious Diseases, the Compound Library of the National Institutes of Health Molecular Library Screening Center, The ChemBridge Library, the ChemDiv Library, or the MayBridge Library.

In certain embodiments, the step of contacting the gene-repressed microbial organism with the candidate prodrug antibiotic compound involves simultaneously contacting a plurality of distinct overexpressing microbial organisms that are overexpressing distinct genes but are otherwise isogenic. In particular embodiments, the plurality of distinct gene-overexpressing microbial organisms that are overexpressing distinct genes but are otherwise isogenic is a microbial organism that transformed with a cDNA library expressing sense cDNA derived from the same microbial organism.

In still another aspect, the invention provides a method of identifying a compound with prodrug antibiotic activity which includes the steps of contacting a microbial organism that is mutant or deficient in multidrug pump efflux; detecting a level of growth of the microbial organism that is mutant or deficient in multidrug pump efflux in the presence of the candidate prodrug antibiotic compound; and comparing the level of growth of the microbial organism that is mutant or deficient in multidrug pump efflux in the presence of the candidate prodrug antibiotic compound to the level of growth of a wild-type microbial organism in the presence of the candidate prodrug antibiotic compound, the wild-type microbial organism not being mutant or deficient in multidrug pump efflux. By this method of the invention, a level of growth, in the presence of the candidate compound, of the multidrug pump efflux mutant or deficient microbial organism that is about equal to (i.e., substantially the same) the level of growth in the presence of the candidate compound of the wild-type microbial organism is indicative of a prodrug antibiotic activity of the candidate compound.

In some embodiments, the microbial organism is a bacterium. In certain embodiments the bacterium is a strain of Escherichia coli (e.g., one that is mutant or deficient in a multidrug pump efflux gene such as AcrA, AcrB, or TolC). In other embodiments the bacteria is a strain of Salmonella typhimurium (e.g., one that is mutant or deficient in a multidrug pump efflux gene such as AcrA, AcrB, or TolC). In still other embodiments the bacterium is a strain of Staphylococcus aureus (e.g., one that is mutant or deficient in a multidrug pump efflux gene such as NorA, NorB, or MepA). In other embodiments, the microbial organism is a fungal organism (e.g., a yeast such as Candida albicans or Saccharomyces cerevisiae). In certain embodiments the fungus has a mutation in or is deficient in a multidrug pump efflux gene such as Pdr5, Mdr1, Cdr1, Cdr2, Cdr3, and Flu1. In particular embodiments, the mutant microbial organism has a loss-of-function mutation in one or more multidrug pump efflux genes. In certain embodiments, the loss-of-function mutation is a null mutation. In other embodiments, the mutant or deficient microbial organism has a conditional mutation in a multidrug pump efflux gene (e.g., a a temperature-sensitive mutation in a multidrug pump efflux gene).

In further embodiments, the mutant or deficient microbial organism is repressed in a multidrug pump efflux gene using a composition such as an antisense oligonucleotide, a ribozyme, a small interfering RNA, or an aptamer. In other embodiments, the multidrug pump efflux gene's activity is regulated by a temperature sensitive mutation or by an inducible promoter or other regulatory element. In particular embodiments, the mutant or deficient microbial organism is repressed in a multidrug pump efflux gene using antisense to a multidrug pump efflux gene. In further particular embodiments, the antisense is produced by a partial or complete cDNA of a multidrug pump efflux gene cloned behind a promoter in the antisense orientation. In other embodiments, the mutant or deficient microbial organism is repressed in a multidrug pump efflux function using a multidrug pump efflux inhibitor such as reserpine, rescinnamine, verapamil, MC207-110, INF 55, INF 271, or Phe-Arg-β-naphthylamide. In further embodiments, the multidrug pump efflux inhibitor is an arylpiperazine, a selective serotonin reuptake inhibitor, a thioxanthese antipsychotic drug or a phenothiazene antipsychotic drug.

In further embodiments, the prodrug antibiotic activity kills or inhibits the growth of a pathogenic organism. In some embodiments, the pathogenic organism is a bacterium. In particular embodiments, the bacterium belong to various Gram-positive and Gram-negative bacterial strains such as Bacillus, Burkholderia, Enterobacter, Escherichia, Helicobacter, Klebsiella, Mycobacterium, Neisseria, Pseudomonas, Staphylococcus, Streptococcus, and Yersinia including drug resistant strains thereof. Non-limiting examples of bacterial pathogenic organisms include Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcusfaecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, and Yersinia pestis. In still further embodiments, the prodrug antibiotic activity kills or inhibits the growth of a pathogenic fungal organism such as Candida albicans, Candida glabrata, Aspergillus niger, Aspergillus fumigatus, Cryptococcus neoformans, and Pneumocystis carinii. In other embodiments, the prodrug antibiotic activity kills or inhibits the growth of a pathogenic helminth (e.g., flatworms (flukes and tapeworms) and roundworms). In further embodiments, the prodrug antibiotic activity kills or inhibits the growth of a pathogenic protozoan organism such as Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, and Microsporidia.

In particular embodiments, the level of microbial growth is determined in a liquid growth medium. In other embodiments, the level of microbial growth is determined in a plate assay. In yet further particular embodiments, the candidate prodrug antibiotic compound is one found in a chemical library such as the Compound Library of The New England Regional Center of Excellence for Biodefense and Emerging Infectious Diseases, the Compound Library of the National Institutes of Health Molecular Library Screening Center, The ChemBridge Library, the ChemDiv Library, or the MayBridge Library.

In some embodiments, the method is a a primary screen for prodrug antibiotic compounds. In other embodiments, the method is a secondary confirmatory screen for prodrug antibiotic activity. In particular embodiments, the primary screen for prodrug antibiotic activity is used in conjunction with a secondary screen according to this aspect of the invention.

In still another aspect, the invention provides prodrug antibiotic compounds identified by any of the above-described methods of the invention. In another aspect, the invention provides methods of inhibiting the growth of, or killing, a pathogen by administering a prodrug compound identified by any of the above-described methods of the invention. In yet another aspect, the invention provides pharmaceutical formulations that contain a prodrug antibiotic compound, identified by any of the above-described methods of the invention, in combination with a pharmaceutically effective carrier. In still another aspect, the invention provides methods of treating a disease (e.g., an infectious disease) comprising administering an effective amount of a prodrug antibiotic compound, identified by any of the above-described methods of the invention, to a subject in need thereof. In another aspect, the invention provides methods of treating an infection by a pathogen comprising administering an effective amount of a prodrug antibiotic compound, identified by any of the above-described methods of the invention, to a subject in need thereof.

In one aspect, the invention provides prodrug compounds of the Formula I: 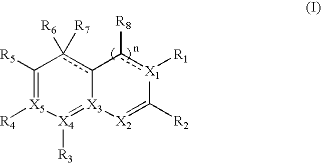
and pharmaceutically acceptable salts, hydrates, and solvates thereof, wherein

R¹ is null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups;

R₂ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl, 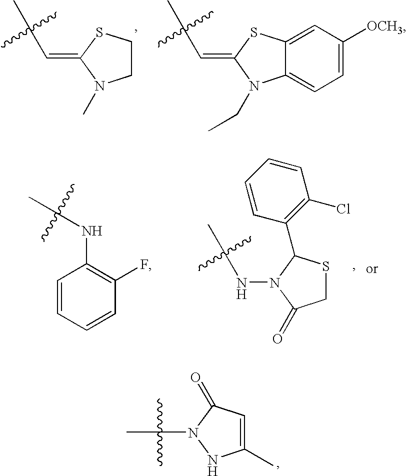
wherein all except —H can be substituted with 0-5 Rₐ groups; or R₁ and R₂ can be taken together to form a 6-membered aryl moiety that can be substituted with 0-4 Rₐ groups;

R₃ and R₄ are each independently null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups; or R₃ and R₄ can be taken together to form a 6-membered aryl moiety that can be substituted with 0-4 Rₐ groups;

R₅ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups; or R₄ and R₅ can be taken together to form a 6-membered aryl moiety that can be substituted with 0-4 Rₐ groups; R₆ and R₇ are each —H, or both R₆ and R₇ can be taken together to form a carbonyl;

R₈ is null, —H, halogen, amino, hydroxyl, cyano, C₁₋₆ alkyl, C₂₋₆ alkenyl, C₂₋₆ alkynyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, heteroarylalkyl,

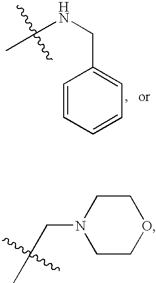

wherein all except H can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃alkyl;

X₁, X₂, X₃, X₄, and X₅ are each independently —N—, —N⁻—, —C(R₁)—, or —C(H)—;

[symbol] denotes a single or double bond;

n is 0 or 1;

x is 0, 1, or 2; and

wherein the prodrug compound is not 1-(benzylamino)-3-(3,6-dichloro-9H-carbazol-9-yl)propan-2-ol, 7-(2-(2-chlorophenyl)-4-oxothiazolidin-3 -ylamino)-1-ethyl-6-fluoro-4-dihydroquinoline-3-carboxylic acid, 1-(3,4-dichlorobenzyl)-1H-benzo [d]imidazol-2-amine, 7-bromo-5-(morpholinomethyl)quinolin-8-ol, 2-(1H-benzo[d]imidazol-2-yl)-5-methyl-1H-pyrazol-3(2H)-one, N⁴-benzyl-N²-(2-fluorophenyl)quinazoline-2,4-diamine, N-(5-ethyl-[1,2,4]triazolo[1,5-c]quinazolin-2-yl)-2,2,2-trifluoroacetamide, (Z)-3 -ethyl-6-methoxy-2-((1,6-dimethylquinolinium-2-yl)methylene)-2,3-dihydrobenzo[d]thiazole, (Z)-2-((1-ethyl-6-methoxy-4-methylbenzo[h]quinolinium-2-yl)methylene)-3-methylthiazolidine, or 1-[2-(4-chlorophenoxy)ethyl]-1H-benzimidazole.
In another aspect, the invention provides prodrug compounds of Formula II: 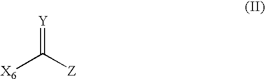
and pharmaceutically acceptable salts, hydrates, and solvates thereof,
wherein

X₆ is NR₉R₁₀, or SR₁₁;

Y is NH, O, or S;

Z is NR₁₂R₁₃;

R₉, R₁₀, R₁₂, and R₁₃, are each independently —H, —OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl-C₁₋₃ alkyl, wherein all except H can be substituted with 0-5 Rₐ groups; or R₁₂ and R₁₃ can be taken together with the nitrogen to which they are attached to form a nitrogen containing 5- or 6-membered monocyclic heterocycle that can be substituted with 0-5 Rₐ groups;

R₁₁ is —H, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, or

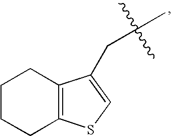

wherein all except H can be substituted with 0-5 Rₐ groups; or R₁₁ and R₁₂ can be taken together to form a 5- or 6-membered monocyclic heterocycle that can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O) C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;

x is 0, 1, or 2; and

wherein the prodrug compound is not 1-(4-chlorophenyl)-1-hydroxy-3-phenylurea, (4,5,6,7-tetrahydrobenzo[b]thiophen-3-yl)methyl carbamimidothioate, 5-ethyltetrahydro-3-(phenylmethyl)-2H-1,3,5-thiadiazine-2-thione, N-(3-chlorophenyl)-3-[(3-chlorophenyl)methyl]tetrahydro-1(2H)-pyrimidinecarbothioamide, or N,N′-bis(3-chlorophenyl)-thiourea.

In another aspect, the invention provides prodrug compounds of Formula III: 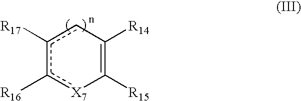
and pharmaceutically acceptable salts, hydrates, and solvates thereof, wherein

R₁₄, R₁₅, R₁₆ and R₁₇ are each independently —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl, or 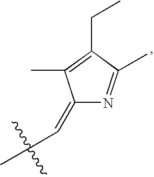
wherein all except H can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O) C ₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;

X₇ is NH, S, O, or O⁺;

[symbol] denotes a single or double bond whereby no more than two of [symbol] can be a double bond;

n is 0 or 1;

x is 0, 1, or 2; and

wherein the prodrug compound is not 2,6-bis(4-(dimethylamino)phenyl)pyrylium,

(E)-3-ethyl-5-((4-ethyl-3,5-dimethyl-2H-pyrrol-2-ylidene)methyl)-2,4-dimethyl-1H-pyrrole, dimethyl 3,4-dihydroxythiophene-2,5-dicarboxylate, or 5-bromo-N-phenyl-2-thiophenecarboxamide.

In one embodiment, the invention provides analogs of prodrug Compound 1 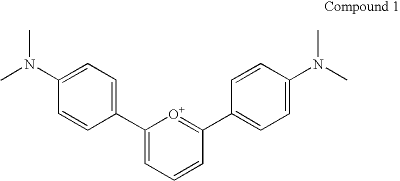
and pharmaceutically acceptable salts, hydrates, and solvates thereof,

wherein any one or more of —H, and —N(CH₃)₂ can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and wherein the analog is not 2,6-bis(4-(dimethylamino)phenyl)pyrylium.
In one embodiment, the invention provides analogs of prodrug Compound 2 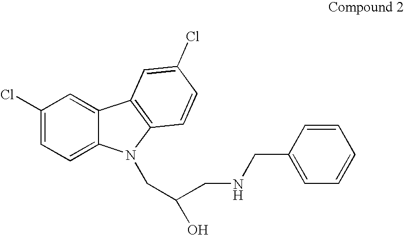
and pharmaceutically acceptable salts, hydrates, and solvates thereof, wherein any one or more of —H, —OH, —Cl, and —CH₂CH(OH)CH₂N(H)benzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 1-(benzylamino)-3-(3,6-dichloro-9H-carbazol-9-yl)propan-2-ol.
In one embodiment, the invention provides analogs of prodrug Compound 3 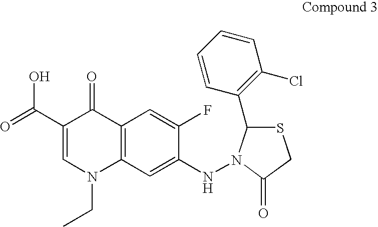
and pharmaceutically acceptable salts, hydrates, and solvates thereof,

wherein any one or more of —H, —F, —CH₂CH₃, —C(O)OH, and chlorophenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 7-(2-(2-chlorophenyl)-4-oxothiazolidin-3-ylamino)-1-ethyl-6-fluoro-4-oxo-1,4-dihydroquinoline-3-carboxylic acid.
In one embodiment, the invention provides analogs of prodrug Compound 4 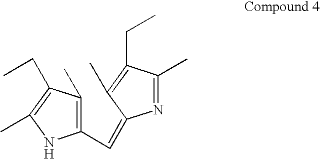
and pharmaceutically acceptable salts, hydrates, and solvates thereof,

wherein any one or more of —H, —CH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; the E configuration can be Z, and wherein the analog is not (E)-3-ethyl-5-((4-ethyl-3,5-dimethyl-2H-pyrrol-2-ylidene)methyl)-2,4-dimethyl-1H-pyrrole.
In one embodiment, the invention provides analogs of prodrug Compound 5 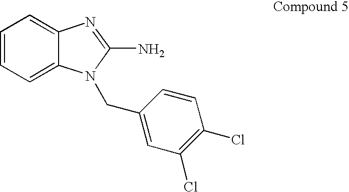
and pharmaceutically acceptable salts, hydrates, and solvates thereof,

wherein any one or more of —H, —NH₂, and dichlorobenzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 1-(3,4-dichlorobenzyl)-1H-benzo[d]imidazol-2-amine.
In one embodiment, the invention provides analogs of prodrug Compound 6 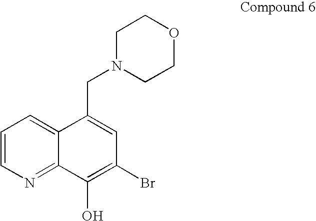
and pharmaceutically acceptable salts, hydrates, and solvates thereof,

wherein any one or more of —H, —Br, —CH₂-morpholino, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 7-bromo-5-(morpholinomethyl)quinolin-8-ol.
In one embodiment, the invention provides analogs of prodrug Compound 7 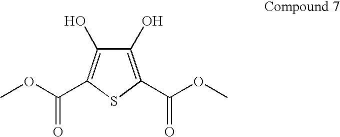
and pharmaceutically acceptable salts, hydrates, and solvates thereof,

wherein any one or more of —H, —OH, —C(O)OCH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; the thiophenyl —S— can be substituted with —O—, and wherein the analog is not dimethyl 3,4-dihydroxythiophene-2,5-dicarboxylate.

In one embodiment, the invention provides analogs of prodrug Compound 8 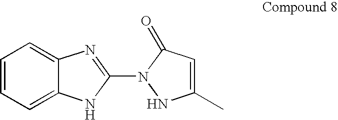
and pharmaceutically acceptable salts, hydrates, and solvates thereof,

wherein any one or more of —H, and —CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 2-(1H-benzo[d]imidazol-2-yl)-5-methyl-1H-pyrazol-3(2H)-one.
In one embodiment, the invention provides analogs of prodrug Compound 9 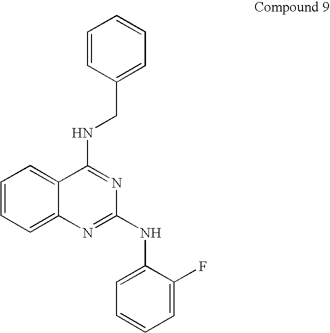
and pharmaceutically acceptable salts, hydrates, and solvates thereof,

wherein any one or more of —H, —F, benzyl, and phenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not N⁴-benzyl-N²-(2-fluorophenyl)quinazoline-2,4-diamine.

In one embodiment, the invention provides analogs of prodrug Compound 10 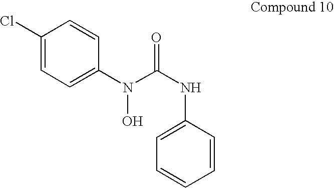
and pharmaceutically acceptable salts, hydrates, and solvates thereof,

wherein any one or more of —H, —OH, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; —C(O) can be substituted with —C(S), and wherein the analog is not 1-(4-chlorophenyl)-1-hydroxy-3-phenylurea.
In one embodiment, the invention provides analogs of prodrug Compound 11 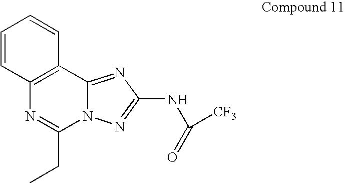
and pharmaceutically acceptable salts, hydrates, and solvates thereof,

wherein any one or more of —H, —F, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not N-(5-ethyl-[1,2,4]triazolo[1,5-c]quinazolin-2-yl)-2,2,2-trifluoroacetamide. In one embodiment, the invention provides analogs of prodrug Compound 12 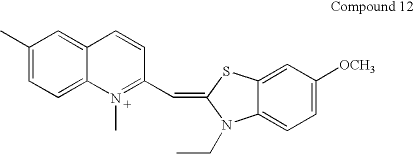
and pharmaceutically acceptable salts, hydrates, and solvates thereof,

wherein any one or more of —H, —CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; the Z configuration can be E, and wherein the analog is not (Z)-3-ethyl-6-methoxy-2-((1,6-dimethylquinolinium-2-yl)methylene)-2,3-dihydrobenzo[d]thiazole.

In one embodiment, the invention provides analogs of prodrug Compound 13 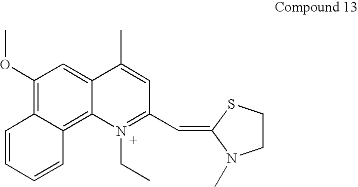
and pharmaceutically acceptable salts, hydrates, and solvates thereof,

wherein any one or more of —H, CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; the Z configuration can be E, and wherein the analog is not (Z)-2-((1-ethyl-6-methoxy-4-methylbenzo[h]quinolinium-2-yl)methylene)-3-methylthiazolidine.
In one embodiment, the invention provides analogs of prodrug Compound 14 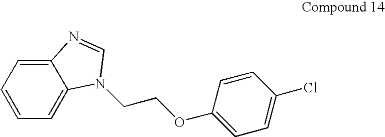
and pharmaceutically acceptable salts, hydrates, and solvates thereof,

wherein any one or more of —H, and —Cl, can be substituted with any one of the following substituents: —H; halogen; —NO₂; —H₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 1-[2-(4-chlorophenoxy)ethyl]-1H-benzimidazole.
In one embodiment, the invention provides analogs of prodrug Compound 15 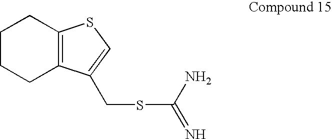
and pharmaceutically acceptable salts, hydrates, and solvates thereof,

wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not (4,5,6,7-tetrahydrobenzo[b]thiophen-3-yl)methyl carbamimidothioate.
In one embodiment, the invention provides analogs of prodrug Compound 16 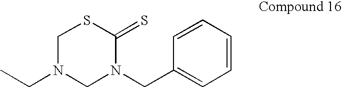
and pharmaceutically acceptable salts, hydrates, and solvates thereof,

wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 5-ethyltetrahydro-3-(phenylmethyl)-2H-1,3,5-thiadiazine-2-thione.
In one embodiment, the invention provides analogs of prodrug Compound 17 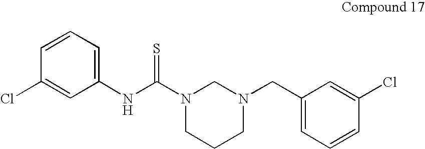
and pharmaceutically acceptable salts, hydrates, and solvates thereof,

wherein any one or more of —H, —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; chlorophenyl can be substituted with heteroaryl, chlorobenzyl can be substituted with alkyl, alkylheteroaryl, and acyl, and wherein the analog is not N-(3-chlorophenyl)-3-[(3-chlorophenyl)methyl]tetrahydro-1(2H)-pyrimidinecarbothioamide.
In one embodiment, the invention provides analogs of prodrug Compound 18 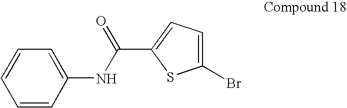
and pharmaceutically acceptable salts, hydrates, and solvates thereof, wherein any one or more of —H, and —Br, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; —C(O) can be substituted with —C(S), S can be substituted with O, and wherein the analog is not 5-bromo-N-phenyl-2-thiophenecarboxamide.
In one embodiment, the invention provides analogs of prodrug Compound 19 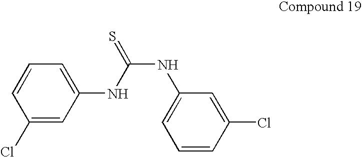
and pharmaceutically acceptable salts, hydrates, and solvates thereof,

wherein any one or more of —H, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; —C(S) can be substituted with —C(O), and wherein the analog is not N,N′-bis(3-chlorophenyl)-thiourea.

In some aspects the invention provides methods of inhibiting the growth of, or killing, a pathogen, the method comprising contacting the pathogen with one or more prodrug compounds of Formulae I, II, and III, 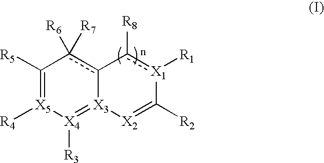
or a pharmaceutically acceptable salt, hydrate, or solvate thereof,
wherein

R¹ is null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups;

R₂ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl, 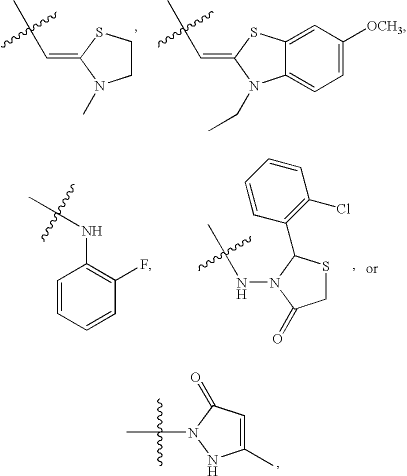
wherein all except —H can be substituted with 0-5 Rₐ groups; or R₁ and R₂ can be taken together to form a 6-membered aryl moiety that can be substituted with 0-4 Rₐ groups;

R₃ and R₄ are each independently null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups; or R₃ and R₄ can be taken together to form a 6-membered aryl moiety that can be substituted with 0-4 Rₐ groups;

R₅ is —H, halogen, amino, hydroxyl, cyano, C₁₋₆ alkyl, C₂₋₆ alkenyl, C₂₋₆ alkynyl, C ₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups; or R₄ and R₅ can be taken together to form a 6-membered aryl moiety that can be substituted with 0-4 Rₐ groups;

R₆ and R₇ are each —H, or both R₆ and R₇ can be taken together to form a carbonyl;

R₈ is null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, heteroarylalkyl,

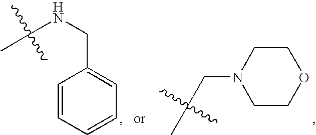

wherein all except H can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O) C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;

X₁, X₂, X₃, X₄, and X₅ are each independently —N—, —N⁺—, —C(R₁)—, or —C(H)—;

[symbol] denotes a single or double bond;

n is 0 or 1; and

x is 0, 1,or 2; 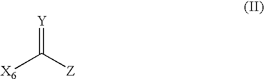
or a pharmaceutically acceptable salt, hydrate, or solvate thereof,
wherein

X₆ is NR₉R₁₀, or SR₁₁;

Y is NH, O, or S;

Z is NR₁₂R₁₃;

R₉, R₁₀, R₁₂, and R₁₃, are each independently —H, —OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, or C₃₋₆ cycloalkyl-C₁₋₃ alkyl, wherein all except H can be substituted with 0-5 Rₐ groups; or R₁₂ and R₁₃ can be taken together with the nitrogen to which they are attached to form a nitrogen containing 5- or 6-membered monocyclic heterocycle that can be substituted with 0-5 Rₐ groups;

R₁₁ is —H, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, or

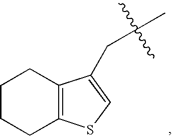

wherein all except H can be substituted with 0-5 Rₐ groups; or R₁₁ and R₁₂ can be taken together to form a 5- or 6-membered monocyclic heterocycle that can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O) C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, NHSO₂C₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;

x is 0, 1, or 2; 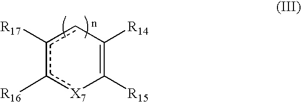
or a pharmaceutically acceptable salt, hydrate, or solvate thereof,
wherein

R₁₄, R₁₅, R₁₆ and R₁₇ are each independently —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl, or 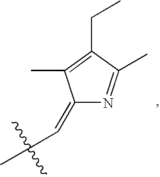
wherein all except H can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;

X₇ is NH, S, O, or O⁺;

[symbol] denotes a single or double bond whereby no more than two of [symbol] can be a double bond;

n is 0 or 1;

x is 0, 1, or 2;

wherein contacting the pathogen with one or more prodrug compounds of Formulae I, II, and III inhibits the growth of, or kills, a pathogen. In certain embodiments of this aspect, the prodrug compound is a compound having Formula I. In other embodiments of this aspect, the prodrug compound is a compound having Formula II. In yet other embodiments of this aspect, the prodrug compound is a compound having Formula III. In certain embodiments of this aspect, the pathogen is selected from the group consisting of a bacterium, a fungus, a protozoan, a helminth, and a combination thereof. In some embodiments of this aspect, the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

In other aspects the invention provides methods of treating an infection by a pathogen in a patient in need thereof, the method comprising administering to the patient an effective amount of one or more prodrug compounds of Formulae I, II, and III, 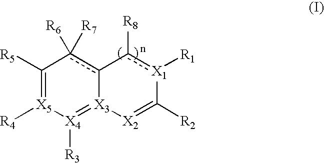
or a pharmaceutically acceptable salt, hydrate, or solvate thereof,
wherein

R¹ is null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylal wherein all except H can be substituted with 0-5 Rₐ groups;

R₂ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl, 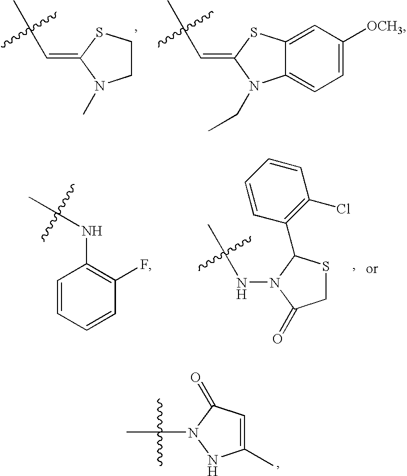
wherein all except —H can be substituted with 0-5 Rₐ groups; or R₁ and R₂ can be taken together to form a 6-membered aryl moiety that can be substituted with 0-4 Rₐ groups;

R₃ and R₄ are each independently null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups; or R₃ and R₄ can be taken together to form a 6-membered aryl moiety that can be substituted with 0-4 Rₐ groups;

R₅ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups; or R₄ and R₅ can be taken together to form a 6-membered aryl moiety that can be substituted with 0-4 Rₐ groups;

R₆ and R₇ are each —H, or both R₆ and R₇ can be taken together to form a carbonyl;

R₈ is null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, heteroarylalkyl, 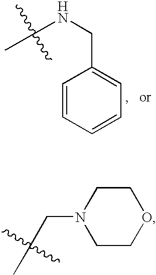
wherein all except H can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O) C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;

X₁, X₂, X₃, X₄, and X₅ are each independently —N—, —N⁺—, —C(R₁)—, or —C(H)—;

[symbol] denotes a single or double bond;

n is 0 or 1; and

x is 0, 1, or 2; 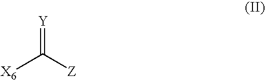
or a pharmaceutically acceptable salt, hydrate, or solvate thereof,
wherein

X₆ is NR₉R₁₀, or SR₁₁;

Y is NH, O, or S;

Z is NR₁₂R₁₃;

R₉, R₁₀, R₁₂, and R₁₃, are each independently —H, —OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, or C₃₋₆ cycloalkyl-C₁₋₃ alkyl, wherein all except H can be substituted with 0-5 Rₐ groups; or R₁₂ and R₁₃ can be taken together with the nitrogen to which they are attached to form a nitrogen containing 5- or 6-membered monocyclic heterocycle that can be substituted with 0-5 Rₐ groups;

R₁₁ is —H, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, or

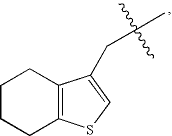

wherein all except H can be substituted with 0-5 Rₐ groups; or R₁₁ and R₁₂ can be taken together to form a 5- or 6-membered monocyclic heterocycle that can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;

x is 0, 1,or 2; 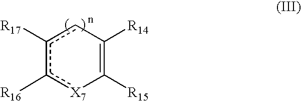
or a pharmaceutically acceptable salt, hydrate, or solvate thereof,
wherein

R₁₄, R₁₅, R₁₆ and R₁₇ are each independently —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl, or 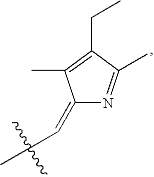
wherein all except H can be substituted with 0-5 Rₐ groups;

Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O) C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl, NHSO₂—C₁₋₆ alkyl, NHSO₂C₃₋₆ cycloalkyl, CO₂C₁₋₆ alkyl, CO₂C₃₋₆ cycloalkyl, CONHC₁₋₆ alkyl; cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;

X₇ is NH, S, O, or O⁺;

[symbol] denotes a single or double bond whereby no more than two of [symbol] can be a double bond;

n is 0 or 1;

x is 0, 1, or 2;

whereby administration of one or more prodrug compounds of Formulae I, II, and III treats the infection by the pathogen. In certain embodiments of this aspect, the prodrug compound is a compound having Formula I. In other embodiments of this aspect, the prodrug compound is a compound having Formula II. In yet other embodiments of this aspect, the prodrug compound is a compound having Formula III. In certain embodiments of this aspect, the pathogen is selected from the group consisting of a bacterium, a fungus, a protozoan, a helminth, and a combination thereof. In some embodiments of this aspect, the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms. In some embodiments, the infection by the pathogen is selected from the group consisting of an upper respiratory tract disease, an infection of a catheter, an infection of an orthopedic prostheses, a urinary tract infection, a gastrointestinal infection, a heart valve infection, endocarditis, a skin infection, a chronic wound, and cystic fibrosis.

In some aspects, the invention provides methods of inhibiting the growth of or eradicating a pathogenic agent by contacting the pathogen with one or more analogs of prodrug Compounds 1-19: 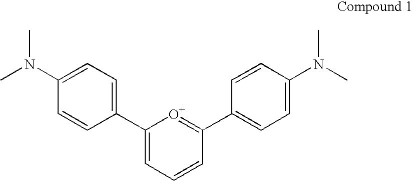
or a pharmaceutically acceptable salt, hydrate, or solvate thereof, wherein any one or more of —H, and —N(CH₃)₂ can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 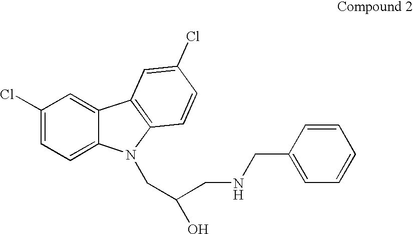
or a pharmaceutically acceptable salt, hydrate, or solvate thereof,

wherein any one or more of —H, —OH, —Cl, and —CH₂CH(OH)CH₂N(H)benzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 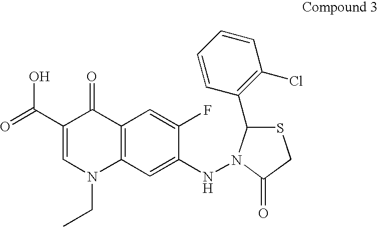
or a pharmaceutically acceptable salt, hydrate, or solvate thereof,

wherein any one or more of —H, —F, —CH2CH3, —C(O)OH, and chlorophenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 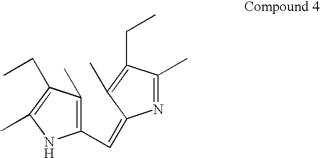
or a pharmaceutically acceptable salt, hydrate, or solvate thereof,

wherein any one or more of —H, —CH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the E configuration can be Z; 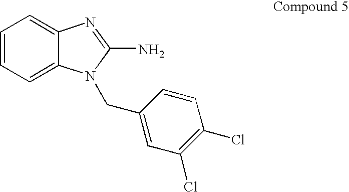
or a pharmaceutically acceptable salt, hydrate, or solvate thereof, wherein any one or more of —H, —NH₂, and dichlorobenzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 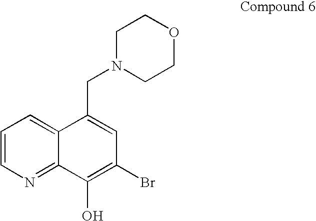
or a pharmaceutically acceptable salt, hydrate, or solvate thereof,

wherein any one or more of —H, —Br, —CH₂-morpholino, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 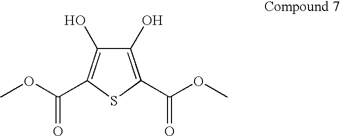
or a pharmaceutically acceptable salt, hydrate, or solvate thereof,

wherein any one or more of —H, —OH, —C(O)OCH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the thiophenyl —S— can be substituted with —O; 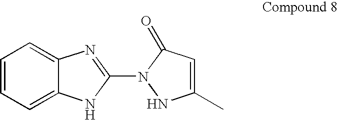
or a pharmaceutically acceptable salt, hydrate, or solvate thereof,

wherein any one or more of —H, and —CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 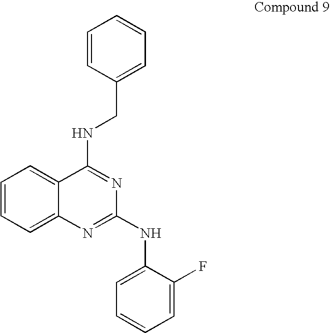
or a pharmaceutically acceptable salt, hydrate, or solvate thereof,

wherein any one or more of —H, —F, benzyl, and phenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 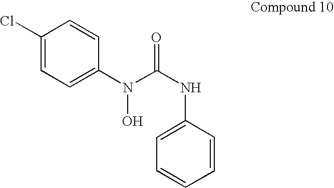
or a pharmaceutically acceptable salt, hydrate, or solvate thereof,

wherein any one or more of —H, —OH, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and —C(O) can be substituted with —C(S); 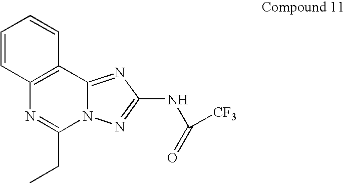
or a pharmaceutically acceptable salt, hydrate, or solvate thereof,

wherein any one or more of —H, —F, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 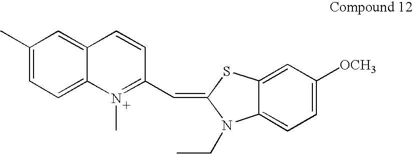
or a pharmaceutically acceptable salt, hydrate, or solvate thereof,

wherein any one or more of —H, —CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the Z configuration can be E; 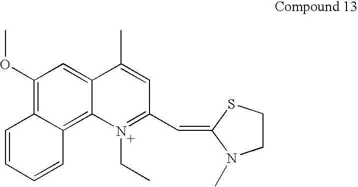
or a pharmaceutically acceptable salt, hydrate, or solvate thereof, wherein any one or more of —H, CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the Z configuration can be E; 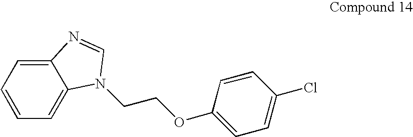
or a pharmaceutically acceptable salt, hydrate, or solvate thereof,

wherein any one or more of —H, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 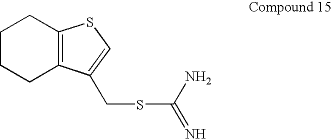
or a pharmaceutically acceptable salt, hydrate, or solvate thereof,

wherein any one or more of the following substituents, —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 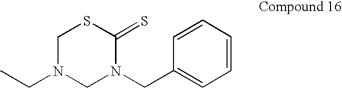
or a pharmaceutically acceptable salt, hydrate, or solvate thereof,

wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 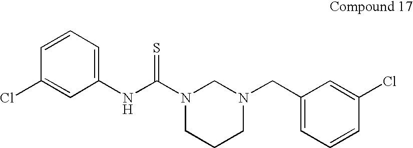
or a pharmaceutically acceptable salt, hydrate, or solvate thereof,

wherein any one or more of —H, —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; chlorophenyl can be substituted with heteroaryl, chlorobenzyl can be substituted with alkyl, alkylheteroaryl, and acyl; 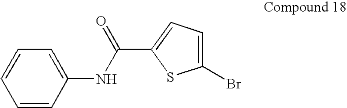
or a pharmaceutically acceptable salt, hydrate, or solvate thereof,

wherein any one or more of —H, and —Br, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; —C(O) can be substituted with —C(S), and S can be substituted with O; 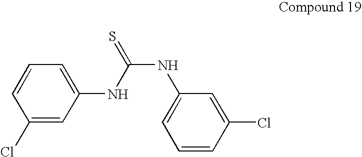
or a pharmaceutically acceptable salt, hydrate, or solvate thereof,

wherein any one or more of —H, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and —C(S) can be substituted with —C(O);

wherein contacting the pathogen with one or more analogs of prodrug Compounds 1-19 inhibits the growth of, or eradicates, the pathogen. In certain embodiments of this aspect, the pathogen is selected from the group consisting of a bacterium, a fungus, a protozoan, a helminth, and a combination thereof. In some embodiments of this aspect, the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

In other aspects, the invention provides methods of treating an infection by a pathogen in a patient in need thereof, the method comprising administering to the patient an effective amount of one or more analogs of prodrug Compounds 1-19: 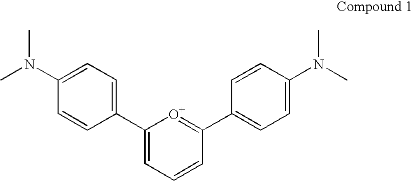
or a pharmaceutically acceptable salt, hydrate, or solvate thereof,

wherein any one or more of —H, and —N(CH₃)₂ can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 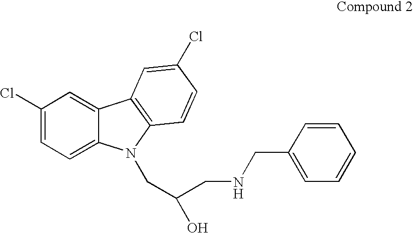
or a pharmaceutically acceptable salt, hydrate, or solvate thereof,

wherein any one or more of —H, —OH, —Cl, and —CH₂CH(OH)CH₂N(H)benzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 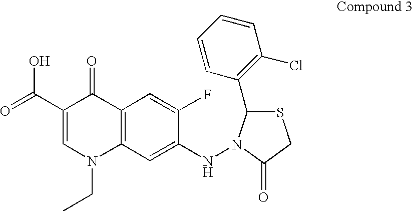
or a pharmaceutically acceptable salt, hydrate, or solvate thereof,

wherein any one or more of —H, —F, —CH2CH3,—C(O)OH, and chlorophenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆ alkyl; C₃₋₆ cycloalkyl; C₃₋₆ cycloalkyl-C₁₋₃ alkyl; alkylaryl; aryl; arylalkyl; heteroaryl; or heteroarylalkyl; 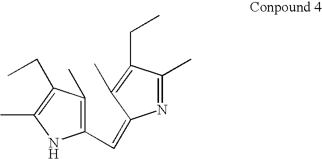
or a pharmaceutically acceptable salt, hydrate, or solvate thereof,

wherein any one or more of —H, —CH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the E configuration can be Z; 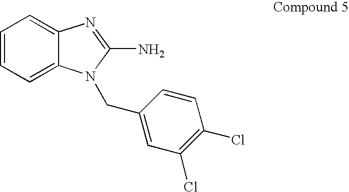
or a pharmaceutically acceptable salt, hydrate, or solvate thereof,

wherein any one or more of —H, —NH₂, and dichlorobenzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 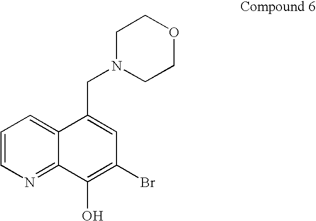
or a pharmaceutically acceptable salt, hydrate, or solvate thereof,

wherein any one or more of —H, —Br, —CH₂-morpholino, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 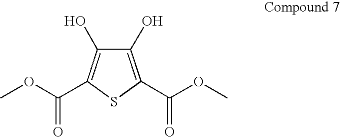
or a pharmaceutically acceptable salt, hydrate, or solvate thereof,

wherein any one or more of —H, —OH, —C(O)OCH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the thiophenyl —S— can be substituted with —O; 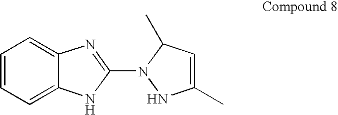
or a pharmaceutically acceptable salt, hydrate, or solvate thereof,

wherein any one or more of —H, and —CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 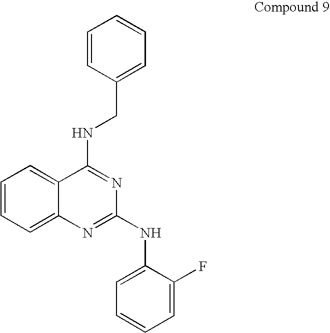
or a pharmaceutically acceptable salt, hydrate, or solvate thereof,

wherein any one or more of —H, —F, benzyl, and phenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 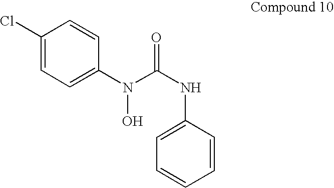
or a pharmaceutically acceptable salt, hydrate, or solvate thereof,

wherein any one or more of —H, —OH, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and —C(O) can be substituted with —C(S); 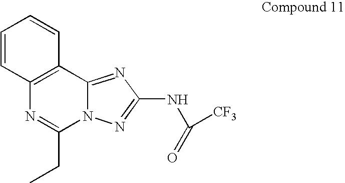
or a pharmaceutically acceptable salt, hydrate, or solvate thereof,

wherein any one or more of —H, —F, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 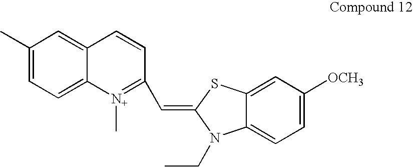
or a pharmaceutically acceptable salt, hydrate, or solvate thereof,

wherein any one or more of —H, —CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the Z configuration can be E; 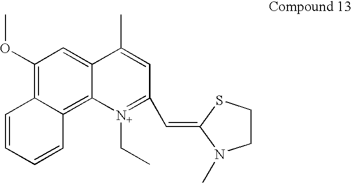
or a pharmaceutically acceptable salt, hydrate, or solvate thereof,

wherein any one or more of —H, CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the Z configuration can be E; 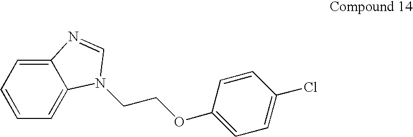
or a pharmaceutically acceptable salt, hydrate, or solvate thereof,

wherein any one or more of —H, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 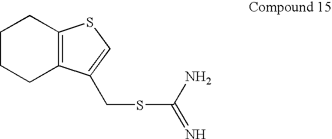
or a pharmaceutically acceptable salt, hydrate, or solvate thereof,

wherein any one or more of the following substituents, —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 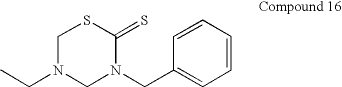
or a pharmaceutically acceptable salt, hydrate, or solvate thereof,

wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 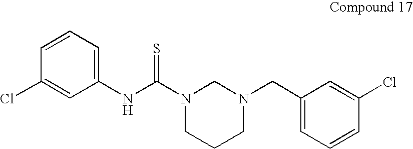
or a pharmaceutically acceptable salt, hydrate, or solvate thereof,

wherein any one or more of —H, —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; chlorophenyl can be substituted with heteroaryl, chlorobenzyl can be substituted with alkyl, alkylheteroaryl, and acyl; 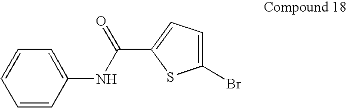
or a pharmaceutically acceptable salt, hydrate, or solvate thereof,

wherein any one or more of —H, and —Br, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; —C(O) can be substituted with —C(S), and S can be substituted with O; 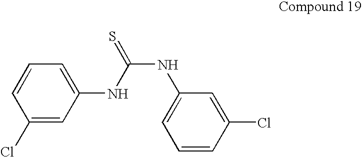
or a pharmaceutically acceptable salt, hydrate, or solvate thereof,

wherein any one or more of —H, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and —C(S) can be substituted with —C(O);

whereby administration of one or more analogs of prodrug Compounds 1-19 treats the pathogenic infection. In certain embodiments of this aspect, the pathogen is selected from the group consisting of a bacterium, a fungus, a protozoan, a helminth, and a combination thereof. In some embodiments of this aspect, the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacterpylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms. In some embodiments, the infection by the pathogen is selected from the group consisting of an upper respiratory tract disease, an infection of a catheter, an infection of an orthopedic prostheses, a urinary tract infection, a gastrointestinal infection, a heart valve infection, endocarditis, a skin infection, a chronic wound, and cystic fibrosis.

In some aspects, the invention provides methods of inhibiting the growth of, or killing a pathogen, the method comprising contacting the pathogen with an effective amount of one or more of prodrug Compounds 1-19: 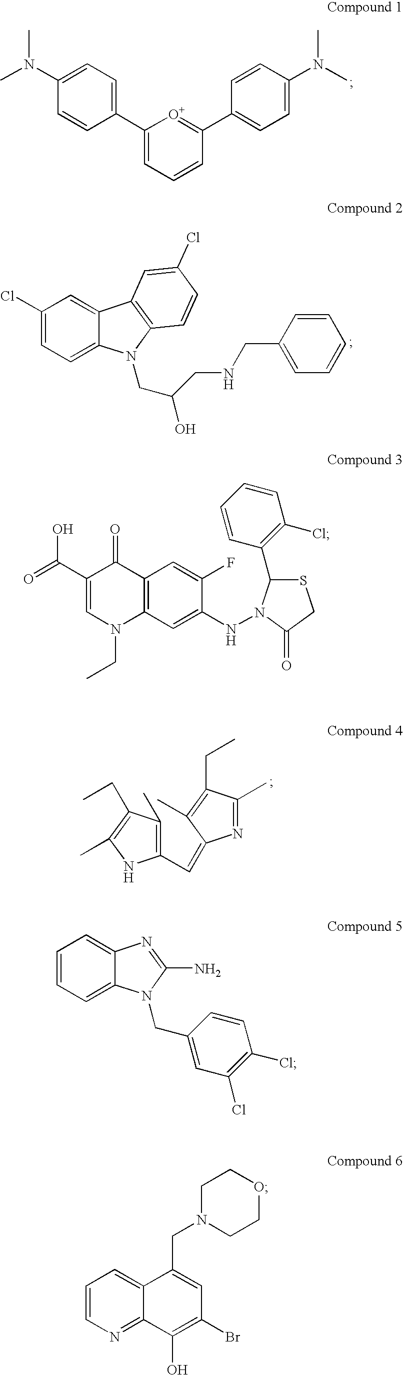 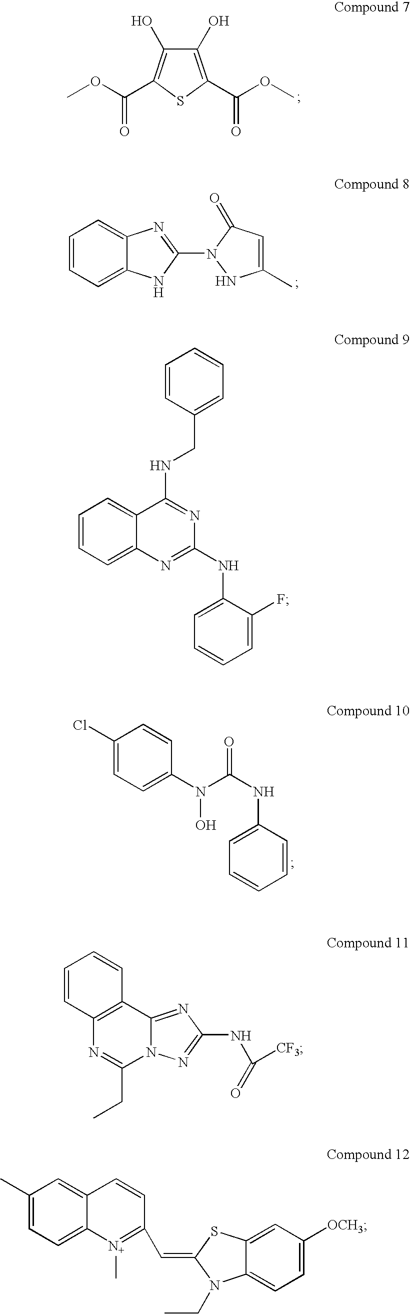 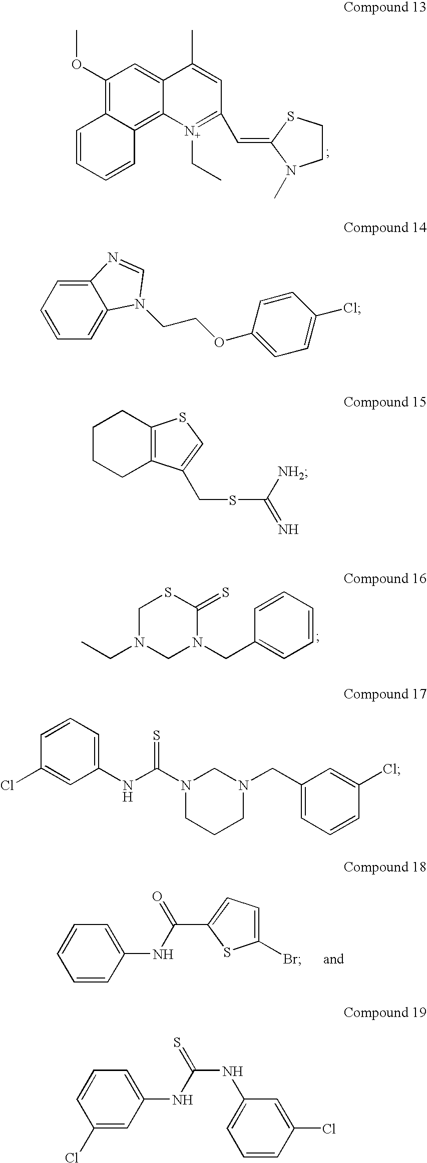
or a pharmaceutically acceptable salt, hydrate, or solvate of Compounds 1-19, wherein contacting the pathogen with one or more of prodrug Compounds 1-19 inhibits the growth of, or kills, the pathogen. In certain embodiments of this aspect, the pathogen is selected from the group consisting of a bacterium, a fungus, a protozoan, a helminth, and a combination thereof. In some embodiments of this aspect, the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

In other aspects, the invention provides methods of treating an infection by a pathogen in a patient in need thereof, the method comprising administering to the patient an effective amount of one or more of prodrug Compounds 1-19: 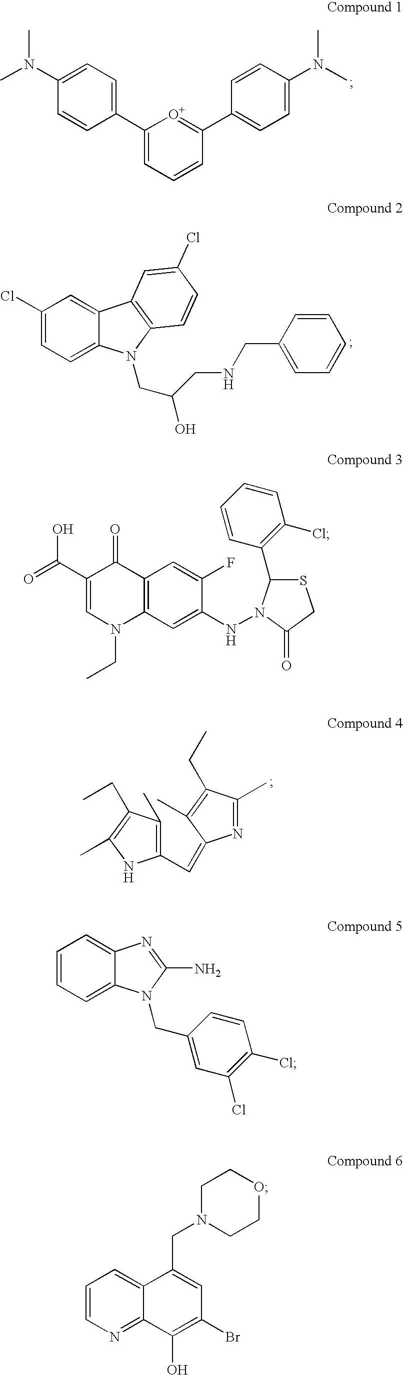 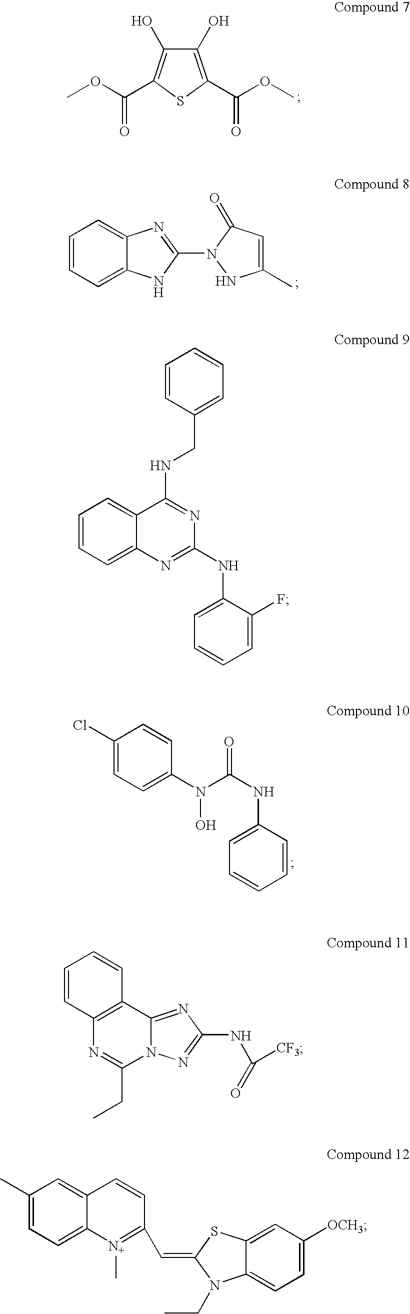 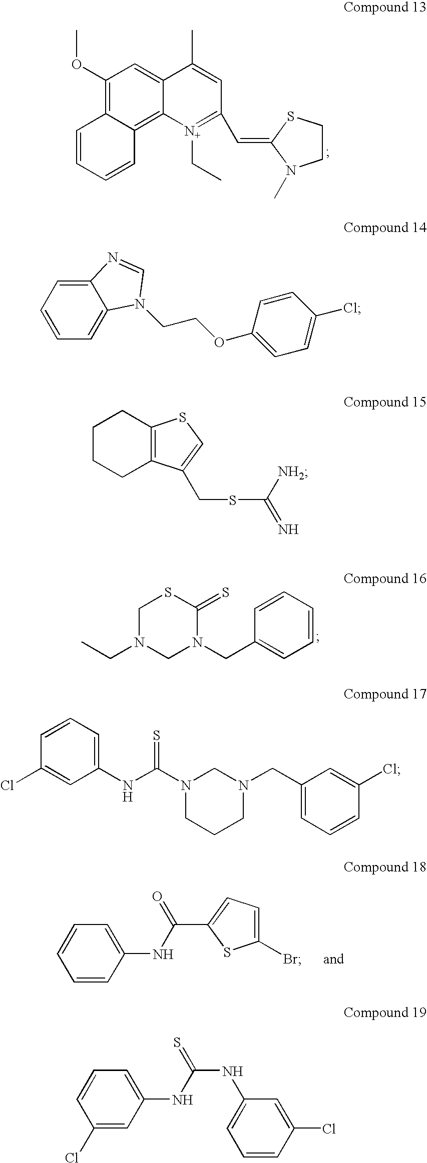
or a pharmaceutically acceptable salt, hydrate, or solvate of Compounds 1-19, whereby administration of one or more of prodrug Compounds 1-19 treats the infection by the pathogen.

In other aspects, the invention provides pharmaceutical compositions comprising compounds or pharmaceutically acceptable salts, hydrates, or solvates of compounds of Formula (I), Formula (II), and Formula (III), and a pharmaceutically acceptable carrier.

In other aspects, the invention provides methods for sterilizing/killing persister cells, comprising contacting the persister cells with an effective amount of a compound or a pharmaceutically acceptable salt of a compound of Formula (I), Formula (II), or Formula (III).

In other aspects, the invention provides pharmaceutical compositions comprising compounds or pharmaceutically acceptable salts, hydrates, or solvates of analogs of Compounds 1-19, and a pharmaceutically acceptable carrier.

In other aspects, the invention provides methods for sterilizing/killing persister cells, comprising contacting the persister cells with an effective amount of an analog of Compounds 1-19 or a pharmaceutically acceptable salt of an analog of Compounds 1-19.

In other aspects, the invention provides pharmaceutical compositions comprising compounds or pharmaceutically acceptable salts, hydrates, or solvates of Compounds 1-19, and a pharmaceutically acceptable carrier.

In other aspects, the invention provides methods for sterilizing/killing persister cells, comprising contacting the persister cells with an effective amount of one or more of Compounds 1-19 or a pharmaceutically acceptable salt of one or more of Compounds 1-19.

### DESCRIPTION OF THE FIGURES

The foregoing and other objects of the present invention, the various features thereof, as well as the invention itself may be more fully understood from the following description, when read together with the accompanying drawings in which:

FIG. 1 is a schematic representation of the properties of an idealized antibiotic agent;

FIG. 2A is a schematic representation of a prodrug screening strategy in which a candidate prodrug is used to contact a microbial cell having reduced (or no) activity of a prodrug activating enzyme;

FIG. 2B is a schematic representation of a prodrug screening strategy in which a candidate prodrug is used to contact a microbial wild type strain;

FIG. 3A is a graphical representation of the level of killing of log-growth, stationary phase and biofilm-phase P. aeruginosa by the bactericidal antibiotic, carbenicillin;

FIG. 3B is a graphical representation of the level of killing of log-growth, stationary phase and biofilm-phase P. aeruginosa by the bactericidal antibiotic, ofloxacin;

FIG. 3C is a graphical representation of the level of killing of log-growth, stationary phase and biofilm-phase P. aeruginosa by the bactericidal antibiotic, tobramycin;

FIG. 3D is a graphical representation of the level of killing of log-growth, stationary phase and biofilm-phase P. aeruginosa by peracetic acid;

FIG. 4 is a schematic representation of the biology of a relapsing biofilm infection;

FIGS. 5A is a schematic representation of the reporter system used to separate persisters from growing cells;

FIG. 5B is a graphical representation of two populations that were detected using forward light-scatter, one that fluoresced brightly (R3), and another that did not (R4);

FIG. 5C are photographical representations of microscopic images of the sorted populations visualized by epifluorescent microscopy (bar, 5 μm);

FIG. 5D is a graphical representation of the survival of cells sorted as described in FIG. 5B and treated with ofloxacin (5 μg/ml) for three hours and then diluted and spotted onto LB agar plates for colony counts;

FIG. 6 is a representation of a heatmap of selected genes expressed in E. coli persister cells;

FIG. 7 is a graphical representation of the properties of a multidrug tolerance of E. coli expressing HipA;

FIG. 8 is a graphical representation of the effects of toxin deletion on persister formation in E. coli;

FIG. 9 is a schematic representation of a model of multidrug tolerance;

FIG. 10 is a graphical representation of the dose-dependent killing of E. coli by metronidazole. Stationary phase cells grown in LB medium in the presence of 1 mM IPTG under anaerobic conditions were challenged for 6 hours with increasing concentrations of metronidazole and then plated on LB agar plates;

FIG. 11A is a schematic representation of the chemical structure of C₂₁H₂₃N₂O⁺, a prodrug antibiotic compounds identified by a prodrug antibiotic screen of the invention;

FIG. 11B is a schematic representation of the chemical structure of C₂₂H₂₀Cl₂N₂O, a prodrug antibiotic compounds identified by a prodrug antibiotic screen of the invention; and

FIG. 12 is a listing of the chemical structure and source of 17 prodrug antibiotic compounds identified by a prodrug antibiotic screen of the invention.

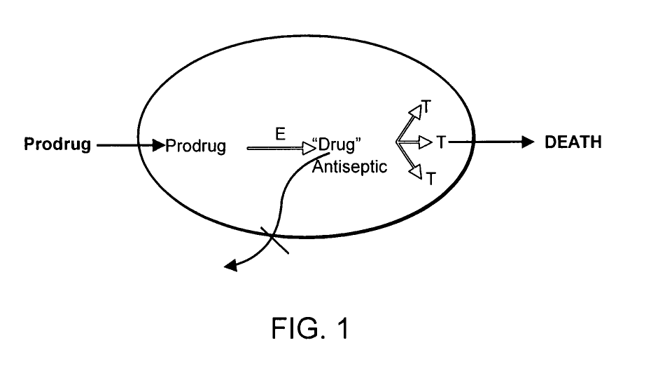

### DETAILED DESCRIPTION OF THE INVENTION

Throughout this application, various publications are referenced. The disclosures of these publications in their entireties are hereby incorporated by reference into this application in order to more fully describe the state of the art as known to those skilled therein as of the date of the invention described and claimed herein. The instant disclosure will govern in the instance that there is any inconsistency between the publications and this disclosure.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

This application relates, in part, to novel methods for drug discovery, drugs identified by these methods, and methods of using these drugs. The methods described herein are based on targeted screens for prodrugs that can function as broad-spectrum antibiotics, sterilizing antibiotics, and/or broad-spectrum sterilizing antibiotics. The screens specifically identify prodrugs that convert into reactive molecules inside a cell of an organism. The activated prodrug then binds to its targets and is irreversibly trapped inside the cell. The activated prodrug is able to bypass efflux by MDR pumps and thus has a broad spectrum of activity. Furthermore, because of its non-specific reactivity, the activated prodrug is able to kill dormant persister cells, leading to a complete sterilization of an infection. The screen also automatically discards generally toxic molecules, a major problem in drug discovery.

Definitions

A “prodrug” is a compound that is converted inside a cell of a pathogen into a reactive molecule which binds to one or more targets and impairs the activity of the cell.

An “antibiotic” is a natural or synthetic compound that inhibits the growth of, or kills, a microorganism (e.g., bacterium, protozoan, fungus). In some instances, the antibiotic is active in inhibiting the growth of or killing other organisms such as helminths.

A “broad-spectrum antibiotic” is an antibiotic that inhibits and/or kills a member of two or more different genuses of a microorganism. For example, an antibiotic that inhibits the growth of and/or kills both E. coli and M. tuberculosis is considered a broad-spectrum antibiotic. Similarly, an antibiotic that kills both S. cerevisiae and C. albicans is considered a broad-spectrum antibiotic.

A “sterilizing antibiotic” is an antibiotic that kills both the growing cells in a population as well as persister cells.

“Persister cells” are antibiotic-tolerant cells produced stochastically by microbial populations.

“Sterilize a microbial population” means to kill a microbial population in the organism the microbe has infected, thereby substantially decreasing or preventing a relapse of the infection by the microbe. For example sterilizing an E. coli O157 population means to kill this pathogenic bacterium in the organism it has infected, thereby reducing or preventing relapse of infection by this pathogen.

“An essential gene” is a gene that is essential to the survival of an organism in a specific environment. Thus, a gene may be essential for survival of a pathogenic organism within the organism it infects (i.e., essential in vivo) but not outside the organism it infects (in vitro).

A population of microbes with “singular mutational identities” means a population of microbes that have mutations in a single genes. For example, a population of bacteria with singular mutational identities means a population of bacteria wherein each bacterium has a mutation in a single gene.

“Alkyl” refers to a hydrocarbon chain that may be a straight chain or branched chain, containing the indicated number of carbon atoms. For example, C₁-C₆ indicates that the group may have from 1 to 6 (inclusive) carbon atoms in it.

“Aryl” refers to cyclic aromatic carbon ring systems made from 6 to 18 carbons. Examples of an aryl group include, but are not limited to, phenyl, napthyl, anthracenyl, tetracenyl, and phenanthrenyl. An aryl group can be unsubstituted or substituted with one or more of the following groups: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Heteroaryl” refers to mono and bicyclic aromatic groups of 4 to 10 atoms containing at least one heteroatom. Heteroatom as used in the term heteroaryl refers to oxygen, sulfur and nitrogen. Examples of monocyclic heteroaryls include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, and pyrimidinyl. Examples of bicyclic heteroaryls include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Arylalkyl” refers to an aryl group with at least one alkyl substitution. Examples of arylalkyl include, but are not limited to, toluenyl, phenylethyl, xylenyl, phenylbutyl, phenylpentyl, and ethylnapthyl. An arylalkyl group can be unsubstituted or substituted with one or more of the following groups: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Heteroarylalkyl” refers to a heteroaryl group with at least one alkyl substitution. A heteroarylalkyl group can be unsubstituted or substituted with one or more of the following: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, isopropyl, n-pentyl, isopentyl, neopentyl, and hexyl.

“C₂-C₆ alkenyl” refers to a straight or branched chain unsaturated hydrocarbon containing 2-6 carbon atoms and at least one double bond. Examples of a C₂-C₆ alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, and isohexene.

“C₃-C₆ alkenyl” refers to a straight or branched chain unsaturated hydrocarbon containing 3-6 carbon atoms and at least one double bond. Examples of a C₃-C₆ alkenyl group include, but are not limited to, propylene, 1-butylene, 2-butylene, isobutylene, sec-butylene, 1-pentene, 2-pentene, isopentene, 1-hexene, 2-hexene, 3-hexene, and isohexene.

“C₂-C₆ alkynyl” refers to a straight or branched chain unsaturated hydrocarbon containing 2-6 carbon atoms and at least one triple bond. Examples of a C₂-C₆ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, and 3-hexyne.

“C₃-C₆ alkynyl” refers to a straight or branched chain unsaturated hydrocarbon containing 3-6 carbon atoms and at least one triple bond. Examples of a C₃-C₆ alkynyl group include, but are not limited to, propyne, 1-butyne, 2-butyne, isobutyne, sec-butyne, 1-pentyne, 2-pentyne, isopentyne, 1-hexyne, 2-hexyne, and 3-hexyne.

“C₁-C₆ alkoxy” refers to a straight or branched chain saturated or unsaturated hydrocarbon containing 1-6 carbon atoms and at least one oxygen atom. Examples of a C₁-C₆ alkoxy include, but are not limited to, methoxy, ethoxy, isopropoxy, butoxy, n-pentoxy, isopentoxy, neopentoxy, and hexoxy.

A “5- to 6-membered monocyclic heterocycle” refers to a monocyclic 5- to 6-membered non-aromatic monocyclic cycloalkyl in which 1-4 of the ring carbon atoms have been independently replaced with a N, O or S atom. When a carbon is replaced by N, the N can be substituted with —H, C₁-C₆ alkyl, or acyl. Representative examples of a 5- to 6-membered monocyclic heterocycle group include, but are not limited to, piperidinyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl.

“Compound of the Invention” as used herein refers to prodrug compounds of Formulae I, II, and III, analogs of prodrug Compounds 1-19, and prodrug Compounds 1-19.

A “subject” is a mammal, e.g., a human, mouse, rat, guinea pig, dog, cat, horse, cow, pig, or non-human primate, such as a monkey, chimpanzee, baboon or rhesus.

The invention also includes pharmaceutical compositions comprising an effective amount of a Compound of the Invention and a pharmaceutically acceptable carrier. The invention includes a Compound of the Invention when provided as a pharmaceutically acceptable prodrug, hydrated salt, such as a pharmaceutically acceptable salt, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

An “effective amount” when used in connection with a Compound of the Invention is an amount effective for treating an infection.

Theoretical Considerations in Antibiotic Prodrug Design

A model antibiotic prodrug is a benign compound that enters into a microbial cell, and is converted by a microbial enzyme into an active, antiseptic-type molecule. This active molecule is more hydrophilic than the prodrug and does not diffuse out of the cell. By the same token, the drug is not a substrate for MDRs that efflux hydrophobic compounds largely based on polarity.

The active molecule binds covalently and non-specifically to one or more “targets” within the cell including, but are not limited to, proteins, peptides, cofactors, DNA and the membrane. The active molecule kills both growing and dormant cells.

There are four known anti-infectives that are prodrugs and resemble the “model antibiotic” described above. These are isoniazid, pyrazinamide, ethionamide, and metronidazole (Table 1).

TABLE 1
Prodrug Antibiotics

| | | Activating | | | Target |
|---|---|---|---|---|---|
| | | enzyme (encoding | | Mechanism | Bacteria/ |
| Molecule | Structure | gene) | Target(s) | of action | Discovery |
| Isoniazid | 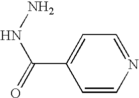 | catalase- peroxidase (katG) (Zhang et al., 1992) | enoyl-acyl carrier protein reductase, fatty acid synthesis (InhA) (Winder and Collins, 1970; Lei et al., 2000) (Larsen et al., 2002); NAD (Vilchese et al., 2005) | Inhibition of synthesis of mycolic acid | M. |
| # tuberculosis1951 | | | | | |
| Pyrazinamide | 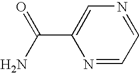 | pyrazinamidase/ nicotinamidase (pnCA) (Hirano et al., 1997) | Non specific(Zhang and Mitchison, 2003) | Cytoplasm acidification/ disruption of membrane functions | M. tuberculosis1952 |
| Ethionamide | 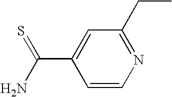 | Monooxygenase (etaA) (DeBarber et al., 2000; Vannelli et al., 2002) | enoyl-acyl carrier protein reductase involved in fatty acid synthesis (InhA) (Larsen et al., 2002); NAD (Vilcheze et al., 2005) | Inhibition of synthesis of mycolic acid | M. tuberculosis |
| # 1956 | | | | | |
| Metronidazole | 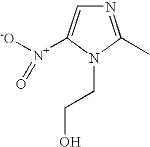 | Nitroreductase (H. pylori rdxA/frxA) (Goodwin et al., 1998; Sisson et al., 2002) | DNA (Edwards, 1977) | | Anaerobic Bacteria 1959 |

The “mechanism of action” of these compounds remains speculative, since the drugs are reactive non-specific molecules capable of hitting more than one target.

Prodrugs were identified in generalized whole-cell screens for antimicrobials inhibiting cell growth, and their prodrug mechanism of action became apparent only after they were introduced into clinical practice. The majority of these compounds were discovered in screens against mycobacteria, and make up the core of anti-M. tuberculosis chemotherapy. M. tuberculosis infection is characterized by slow-growing organisms that can enter into a dormant state highly tolerant to antimicrobials (Stewart et al. (2003) Nat. Rev. Microbiol. 1:97-105, Gomez et al (2004) Tuberculosis (Edinb) 84:29-44). An ability to kill cells, rather than simply inhibit growth, is an essential requirement for tuberculosis drugs. The only prodrug with a fairly broad spectrum is metronidazole, which is converted into an active form in bacterial cells under anaerobic conditions and acts specifically against anaerobic species. Accordingly, there is still no single broad-spectrum prodrug antibiotic available.

All four existing prodrugs were discovered in the 1950's (Table 1). At the time, the total size of libraries available for antiinfective screening was less than equal to 10⁴. The total size of non-redundant, non-combinatorial compound libraries today is 10⁶-10⁷. In some aspects, the instant application provides screens to identify antimicrobial prodrugs from such compound libraries.

Screens to Identify Prodrugs

The screens described herein are useful for identifying prodrug compounds that are converted inside cells of a pathogen into reactive antiseptic molecules that can kill the pathogen and sterilize the infection caused by the pathogen. The rationale is to screen compounds against strains differentially expressing an enzyme potentially capable of activating a prodrug into an active compound. A strain overproducing an activating enzyme is more susceptible to a prodrug than the wild type, whereas a strain with a suppressed activating enzyme is more resistant than the wild type. The screens described herein are a departure from traditional approaches based on disabling a particular protein target. A combination of genomics with high throughput screening (HTS) makes this a straightforward approach. Genomics provides candidate enzymes that can activate prodrugs, and a rational screening design enables efficient identification and validation of hits. Conventional whole cell screens suffer from a high background of non-specifically acting compounds such as membrane-acting or DNA-damaging agents. An attractive feature of the screens described herein is the ability to discard generally toxic compounds, since these will have similar activity against the wild type and strains differentially expressing a prodrug activating enzyme and will be automatically discarded.

a. Screen Using Strains Mutated in an Activating Enzyme

This screen is based on using a microbial organism having a mutation in one or more genes. Among these mutants will be one or more microbial organisms that have mutations in a gene encoding an enzyme that activates a prodrug. The mutation includes, but is not limited to, a loss-of-function mutation, a null mutation, a conditional mutation, or a conditional mutation which is a temperature-sensitive mutation. The mutation may be in an essential gene(s). In ceratin cases, the mutation is in an essential gene in vivo.

In one example, the screen involves contacting a microbial organism that is mutant in one or more genes with a candidate prodrug compound. The screen further involves comparing the level of growth of the mutant microbial organism in the presence of the candidate prodrug compound to the level of growth of a wild type microbial organism in the presence of the candidate prodrug antibiotic compound. A greater level of growth of the mutant microbial organism in the presence of the candidate compound than the level of growth of the wild-type microbial organism in the presence of the candidate compound is indicative of a prodrug activity of the candidate compound. The level of growth of the mutant and wild type organisms can be determined by any method known in the art. In some embodiments, the level of growth is determined in a liquid growth medium. In other embodiments, the level of growth is determined in a plate assay.

The contacting step may be performed with a plurality of mutant microbial organisms that are each mutant in different genes but otherwise isogenic. These mutant microbial strains may be mixed together and the resulting suspension can then be used to contact a candidate prodrug compound. The suspension may be dispensed into wells of a microtiter plate for screening with the candidate prodrug. Growth of cells within a suspension of mutant microbial organisms contacted with a candidate prodrug compound, but not in a suspension of wild type cells contacted with a candidate prodrug, indicates a prodrug hit. If growth occurs in the suspension of mutants, it is because at least one mutant is mutated in a gene encoding a protein that is necessary to convert the candidate prodrug compound into an active drug. Because the prodrug activating enzyme is absent, the prodrug is not converted into its active form and does not kill the cell.

In some cases, resistance may develop against compounds identified by the screen due to null mutations in non-essential activating enzymes. Accordingly, it may be useful to identify prodrug activating enzymes that are essential in vivo. In some cases, the screen identifies prodrug activating enzymes that are essential in vivo (i.e., essential in the organism that the pathogen infects) and therefore not subject to rapid resistance development. In vivo essentiality of a gene of a pathogen within the organism it infects may be determined by any method known in the art. For example, in vivo essentiality of an E. coli gene can be determined by infecting mice with E. coli O157 and following the rate of clearance of knockout mutants: increased clearance indicates essentiality of the gene in vivo.

In certain examples, a secondary screen may then be performed with this prodrug hit compound against each strain of the mutant microbial population dispensed in individual wells, to identify the mutant lacking an activating enzyme for the prodrug. A prodrug has higher activity against a strain expressing an activating enzyme and lower activity against a strain attenuated in this enzyme. This discriminates the prodrug from all other compounds and serves to validate the hits.

b. Screen Using Bacterial Strains Having Diminished Expression of Enzymes

In this version of the screen to identify a prodrug compound, a gene of the microbial organism is repressed. The method involves contacting a microbial organism with a candidate prodrug compound while one or more of its genes is repressed. In certain embodiments the repressed gene is a gene ecoding a prodrug-activating enzyme. The gene's activity may be repressed using an agent including, but not limited to, antisense oligonucleotides, ribozymes, small interfering RNAs, and aptamers. Methods of making antisense oligonucleotides, ribozymes, small interfering RNAs, and aptamers are well known in the art. The gene's activity may also be repressed using temperature sensitive mutations or by regulating expression of the gene, or an activator or repressor of the gene, through an inducible promoter. The gene may be an essential gene in vivo. The level of growth of the gene-repressed microbial organism in the presence of the candidate prodrug antibiotic compound is compared to the level of growth of the same microbial organism in which the one or more genes of the organism is not repressed. A greater level of growth of the gene-repressed microbial organism in the presence of the compound than the level of growth of the non gene-repressed microbial organism in the presence of the compound is indicative of a prodrug antibiotic activity of the candidate compound. In some screens, the step of contacting the gene-repressed microbial organism with the candidate prodrug antibiotic compound comprises simultaneously contacting a plurality of distinct gene-repressed microbial organisms that are repressed in distinct genes but otherwise isogenic.

One or more genes of the microbial organism may be repressed using antisense technology. The antisense molecule for use in this screen may be produced by a partial or complete cDNA cloned behind a promoter in the antisense orientation. In the antisense RNA approach to the screen, a set of E. coli strains with diminished expression of essential enzymes are constructed and used to screen for prodrugs as described above.

c. Screen Using Strains Overexpressing Prodrug Activating Enzymes

This version of the screen for prodrug compounds is based on overexpression of a prodrug activating enzyme in microbial cells. The rationale of the screen is that a microbial cell overexpressing a prodrug activating enzyme is more susceptible to a prodrug than the wild type microbe. In one example with E. coli overexpressing NfnB, the activating enzyme for metronidazole, the overexpression strain showed greater than 50-fold sensitivity as compared to the wild type control (see, Example 4). Metronidazole completely “sterilized” the population of NfnB overexpressing cells—i.e., it eradicated the NfnB overexpressing cells. This is the first observation of sterilization for an antibiotic. This finding also suggests that finding a prodrug with a better fit to its activating enzyme will produce a better therapeutic.

In a specific example, a set of strains from a library overexpressing conserved essential genes coding for potential prodrug-activating enzymes is used for screen development. In order to validate the functionality of the overexpressed recombinant protein, chromosomal disruptions of the gene are created. An ability to make a knockout validates the functional expression of the recombinant protein, and such a strain becomes part of the screening set. In one embodiment, the enzymes share homology to their counterparts in other microbial organisms, and do not have close homologs in humans. Each strain is then screened against a candidate prodrug compound, and a compound showing higher activity in the overexpressing strain as compared to the wild type is identified as a prodrug hit.

d. Screens Using MultiDrug Pump Mutants

This version of the screen for prodrug compounds is based on contacting a microbial organism that is mutant or deficient in multidrug pump efflux with a candidate prodrug compound. Prodrugs activated by prodrug activating enzymes convert into reactive molecules that bind to their targets creating an irreversible sink, thereby inhibiting or preventing multidrug resistance efflux of the activated prodrug. The screen involves comparing the growth of the microbial organism that is mutant or deficient in multidrug pump efflux in the presence of the candidate prodrug antibiotic compound to the level of growth of a wild type microbial organism in the presence of the candidate prodrug antibiotic compound. It is to be understood that the wild type microbial organism is not mutant or deficient in multidrug pump efflux. If the level of growth of the microbial organism that is mutant or deficient in multidrug pump efflux in the presence of the candidate prodrug compound is about equal to the level of growth of the wild type microbial organism in the presence of the compound, the candidate compound is identified as a prodrug compound.

The mutation may be a loss-of-function mutation, a null mutation, or a conditional mutation in a multidrug efflux gene. In some embodiments, the conditional mutation is a temperature-sensitive mutation. If the microbial organism is a bacterium such as Escherichia coli or Salmonella typhimurium, non-limiting examples of multidrug efflux genes include AcrA, AcrB, and TolC. If the microbial organism is a bacterium such as Staphylococcus aureus, non-limiting examples of multidrug efflux genes include NorA, NorB, and MepA. If the microbial organism is a fungus such as Saccharomyces cerevisiae or Candida albicans, non-limiting examples of multidrug efflux genes include Pdr5, Mdr1, Cdr1, Cdr2, Cdr3, and Flu1. In some cases, the microbial organism is made deficient in mutidrug efflux by treating the microbial cell with multidrug pump efflux inhibitors. Non-limiting examples of multidrug pump efflux inhibitors include reserpine, rescinnamine, verapamil, MC207-110, INF 55, INF 271, and PH-Arg-β-naphthylamide.

In all four of the screens described above, the microbial organism includes, but is not limited to bacteria, protozoa, and fungi. Any bacterium, protozoan, or fungus may be used in the screens. Examples of bacteria for use in the screens include, but are not limited to, Escherichia coli, Salmonella typhimurium, Staphylococcus aureus, Pseudomonas aeruginosa, Hemophilus influenza, Mycobacterium tuberculosis, and Enterococcus faecalis. Examples of fungi for use in the screens include, but are not limited to, Saccharomyces cerevisiae, Candida albicans. In all four screens described above, the prodrug compounds identified in the screens can be used to inhibit, reduce, prevent growth of, and/or kill a pathogenic organism. In certain embodiments, the pathogenic organism is a bacterium, a protozoan, a fungus, or a helminth. In some embodiments, the bacteria belong to various Gram-positive and Gram-negative bacteria strains including, but not limited to, Bacillus, Burkholderia, Enterobacter, Escherichia, Helicobacter, Klebsiella, Mycobacterium, Neisseria, Pseudomonas, Staphylococcus, Streptococcus, and Yersinia including drug resistant strains thereof. Non-limiting examples of bacterial pathogenic organisms that can be inhibited or killed by the prodrugs of the screen include, Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Hemophilus influenza, Helicobacterpylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, and Yersinia pestis. Non-limiting examples of pathogenic fungal organisms that can be inhibited or killed by the prodrugs of the screen include, Candida albicans, Candida glabrata, Aspergillus niger, Aspergillus fumigatus, Cryptococcus neoformans, and Pneumocystis carinii. Non-limiting examples of pathogenic protozoan organisms that can be inhibited or killed by the prodrugs of the screen include, Plasmodium falciparum, Plasmodium vivax, Trypanosoma cruzeii, Entameoba histolytica, Entamoeba hartmanii, Dientamoeba fragilis, Giardia lamblia, Cryptosporidiumparvum, Naegleria fowleri, Acanthomeaba SPP, Isospora belli, and Microsporidia. Non-limiting examples of helminthic pathogenic organisms that can be inhibited or killed by the prodrugs of the screen include, flatworms (flukes and tapeworms) and roundworms.

In all four of the screens described above, any candidate prodrug can be assayed. In some embodiments, a candidate prodrug library is used. Nonlimiting examples of candidate prodrug compound libraries include The Compound Library of the New England Regional Center of Excellence for Biodefense and Emergine Infectious Diseases, The Compound Library of the National Institutes of Health Molecular Library Screening Center, The ChemBridge Library, the ChemDiv Library, and the MayBridge Library.

Biofilms and Persisters

Multidrug tolerance of pathogens is in large part the result of the entry of microbial cells into a dormant state. Such dormant cells are likely responsible for latent (chronic) diseases such as, but not limited to, tuberculosis, syphilis, and Lyme disease, which have thus far been suppressed by known antimicrobials, but not eradicated. Because of the importance of developing therapeutics that are capable of killing these dormant cells and thus eradicating infection, the screens described above can be adapted/modified to identify prodrug compounds that have a sterilizing ability against biofilms and persister cells.

Biofilms are bacterial or yeast communities that settle and proliferate on surfaces and are covered by an exopolymer matrix. They are slow-growing and many are in the stationary phase of growth. They can be formed, by most, if not all pathogens. According to the CDC, 65% of all infections in the United States are caused by biofilms that can be formed by common pathogens such as E. coli, P. aeruginosa, S. aureus, E. faecalis, and S. epidermidis. Infections ascribed to biofilms include: childhood middle ear infection and gingivitis; UTI; and infections of indwelling devices such as catheters, heart valves, and orthopedic devices. Biofilm infections also occur in patients with cystic fibrosis. Biofilm infections are highly recalcitrant to antibiotic treatment and adequate therapy against these infections is lacking. While antibiotic treatment will kill most biofilm and planktonic cells, the antibiotics do not kill persisters. The biofilm exopolymer matrix protects against immune cells and persisters that are contained in the biofilm can survive both the onslaught of the antibiotic treatment and the immune system. When antibiotic levels decrease, these persisters can repopulate the biofilm, which will shed off new planktonic cells, producing the relapsing biofilm infection.

Persisters are dormant cells that are tolerant of multiple antibiotics. Bactericidal antibiotics kill cells not by inhibiting its cellular target, but rather by corrupting the target to create a toxic product. For example, aminoglycoside antibiotics kill the cell by interrupting translation, which produces misfolded toxic peptides. Beta-lactam antibiotics, such as penicillin, inhibit peptidoglycan synthesis, which activates autolysin enzymes present in the cell wall leading to digestion of the peptidoglycans and cell death. Fluoroquinolones inhibit the ligase step of DNA gyrase and topoisomerase, without affecting its nicking activity, thereby converting these enzymes into endonucleases. The ability of persister cells to survive killing by antibiotics without expressing or using resistance mechanisms (i.e., tolerance) is mediated by preventing target corruption by a bactericidal agent through the blocking of antibiotic targets. If persisters are dormant and have minimal cell wall synthesis, translation, or topoisomerase activity, then the antibiotics will bind to, but will be unable to corrupt, the function of their targets. In this way, tolerance could enable resistance of persister cells to killing by antibiotics, but at the price of non-proliferation. The simplest method to form a persister cell is through the overproduction of proteins that are toxic to the cell and inhibit growth.

Given the prominent role of tolerance to antibiotics in infectious disease, the need for compounds that can eradicate persisters is clear. Thus, persister cells of a pathogen can be contacted with prodrug candidate compounds obtained from the screens described above to determine if the compounds are active (i.e., kill or eradicate) against persisters. In addition, the prodrug compounds can be tested for their ability to eradicate stationary populations of the pathogen.

Compounds of the Invention

The Prodrug Compounds of Formulae I, II, and III

The present invention provides prodrug compounds according to Formula (1) below: 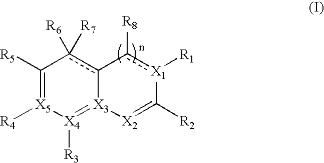
and pharmaceutically acceptable salts, hydrates, and solvates thereof,
wherein

R¹-R⁸, X¹-X₅ and n, are as defined above for the prodrug compounds of Formula (I) and wherein the prodrug compounds are not 1-(benzylamino)-3-(3,6-dichloro-9H-carbazol-9-yl)propan-2-ol, 7-(2-(2-chlorophenyl)-4-oxothiazolidin-3-ylamino)-1-ethyl-6-fluoro-4-oxo-1,4-dihydroquinoline-3-carboxylic acid, 1-(3,4-dichlorobenzyl)-1H-benzo[d]imidazol-2-amine, 7-bromo-5-(morpholinomethyl)quinolin-8-ol, 2-(1H-benzo[d]imidazol-2-yl)-5-methyl-1H-pyrazol-3(2H)-one, N⁴-benzyl-N²-(2-fluorophenyl)quinazoline-2,4-diamine, N-(5-ethyl-[1,2,4]triazolo[1,5-c]quinazolin-2-yl)-2,2,2-trifluoroacetamide, (Z)-3-ethyl-6-methoxy-2-((1,6-dimethylquinolinium-2-yl)methylene)-2,3-dihydrobenzo[d]thiazole, (Z)-2-((1-ethyl-6-methoxy-4-methylbenzo[h]quinolinium-2-yl)methylene)-3-methylthiazolidine, or 1-[2-(4-chlorophenoxy)ethyl]-1H-Benzimidazole.

In one embodiment, n is 0.

In one embodiment, n is 1.

In one embodiment, X₁ is N.

In one embodiment, X₂ is N.

In one embodiment, R₁ and R₂ form an aryl moiety.

In one embodiment, X₃ is N.

In one embodiment, X₅ is N.

In one embodiment, R₂ is an amine.

In one embodiment, X₂ is N⁺.

In one embodiment, R₂ is

In one embodiment, R₂ is

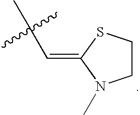

In one embodiment, R₂ is

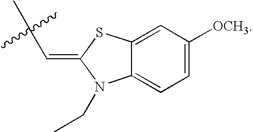

In one embodiment, R₂ is

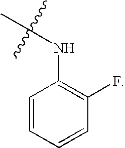

In one embodiment, R₂ is

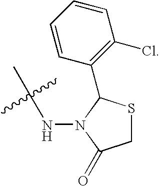

In one embodiment, R₂ is

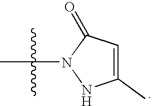

In one embodiment, R₆ and R₇ together form a carbonyl.

In one embodiment, R₈ is an amine.

In one embodiment, [symbol] is double bond.

In one embodiment, X₂ is C(R₁) and R₁ is OH.

In one embodiment, X₂ is C(R₁) and R₁ is —CH₂CH₂O (chlorophenyl).

In one embodiment, X₂ is C(R₁) and R₁ is OH.

The invention also relates to compounds of Formula (II): 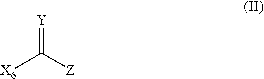
and pharmaceutically acceptable salts, hydrates, and solvates thereof,
wherein

X₆, Y and Z are as defined above for the compounds of Formula (II) and wherein the prodrug compound is not 1-(4-chlorophenyl)-1-hydroxy-3-phenylurea, (4,5,6,7-tetrahydrobenzo[b]thiophen-3-yl)methyl carbamimidothioate, 5-ethyltetrahydro-3-(phenylmethyl)-2H-1,3,5-thiadiazine-2-thione, N-(3-chlorophenyl)-3-[(3-chlorophenyl)methyl]tetrahydro-1(2H)-pyrimidinecarbothioamide, or N,N′-bis(3-chlorophenyl)-thiourea.

In one embodiment, X₆ is NR₉R₁₀.

In one embodiment, X₆ is SR₁₁.

In one embodiment, Y is NH.

In one embodiment, Y is O.

In one embodiment, Y is S.

In one embodiment, R₁₁ is

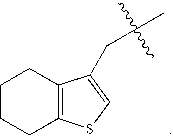

In one embodiment, R₉ and R₁₂ together form an aryl.

In one embodiment, R₁₂ and R₁₃ together form an aryl.

In one embodiment, R₁₁ and R₁₂ together to form a 6-membered monocyclic heterocycle.

In one embodiment, R₁₂ and R₁₃ together to form a 6-membered monocyclic heterocycle.

In one embodiment, R₉ is OH.
The invention also relates to compounds of Formula (III): 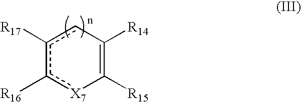
and pharmaceutically acceptable salts, hydrates, and solvates thereof,
wherein

R₁₄-R₁₇, X₇, and n are as defined above for the compounds of Formula (III) and wherein the prodrug compound is not 2,6-bis(4-(dimethylamino)phenyl)pyrylium, (E)-3-ethyl-5-((4-ethyl-3,5-dimethyl-2H-pyrrol-2-ylidene)methyl)-2,4-dimethyl-1H-pyrrole, dimethyl 3,4-dihydroxythiophene-2,5-dicarboxylate, or 5-bromo-N-phenyl-2-thiophenecarboxamide.

In one embodiment, n is 0.

In one embodiment, n is 1.

In one embodiment, X₇ is S.

In one embodiment, X₇ is O⁺.

In one embodiment, X₇ is NH.

In one embodiment, R₁₅ is

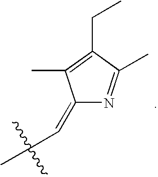

In one embodiment, R₁₅ is halogen.

In one embodiment, R₁₆ is —C(O)NHaryl.

In one embodiment, R₁₅ is aryl.

In one embodiment, R₁₄, R₁₆ and R₁₇ are alkyl.

In one embodiment, R₁₄ is OH.

In one embodiment, R₁₇ is OH.

In one embodiment, R₁₄ and R₁₇ are OH.

In one embodiment, R₁₅ is —(O)OCH3.

In one embodiment, R₁₆ is —(O)OCH3.

In one embodiment, R₁₅ and R₁₆ are —(O)OCH3.

The Analogs of Prodrug Compounds 1-19

The invention also relates to analogs of prodrug Compound 1: 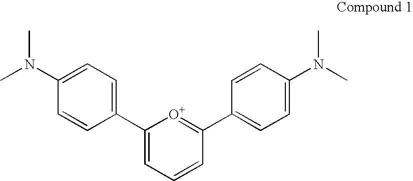
and pharmaceutically acceptable salts, hydrates, and solvates thereof,

wherein any one or more of —H, and —N(CH₃)₂ can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and wherein the analog is not 2,6-bis(4-(dimethylamino)phenyl)pyrylium.

The invention also relates to analogs of prodrug Compound 2 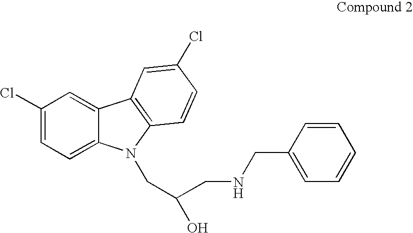
and pharmaceutically acceptable salts, hydrates, and solvates thereof,

wherein any one or more of —H, —OH, —Cl, and —CH₂CH(OH)CH₂N(H)benzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 1-(benzylamino)-3-(3,6-dichloro-9H-carbazol-9-yl)propan-2-ol.

The invention also relates to analogs of prodrug Compound 3 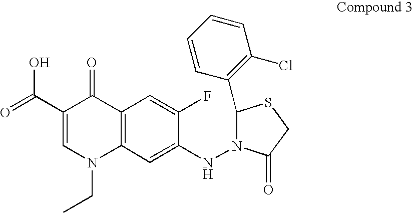
and pharmaceutically acceptable salts, hydrates, and solvates thereof,

wherein any one or more of —H, —F, —CH₂CH₃, —C(O)OH, and chlorophenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆ alkyl; C₃₋₆ cycloalkyl; C₃₋₆ cycloalkyl-C₁₋₃ alkyl; alkylaryl; aryl; arylalkyl; heteroaryl; and wherein the analog is not 7-(2-(2-chlorophenyl)-4-oxothiazolidin-3-ylamino)-1-ethyl-6-fluoro-4-oxo-1,4-dihydroquinoline-3-carboxylic acid.

The invention also relates to analogs of prodrug Compound 4 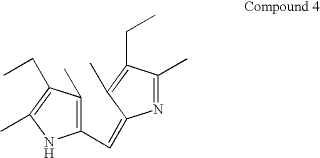
and pharmaceutically acceptable salts, hydrates, and solvates thereof,

wherein any one or more of —H, —CH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; the E configuration can be Z, and wherein the analog is not (E)-3-ethyl-5-((4-ethyl-3,5-dimethyl-2H-pyrrol-2-ylidene)methyl)-2,4-dimethyl-1H-pyrrole.

The invention also relates to analogs of prodrug Compound 5 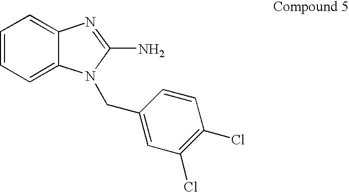
and pharmaceutically acceptable salts, hydrates, and solvates thereof,

wherein any one or more of —H, —NH₂, and dichlorobenzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 1-(3,4-dichlorobenzyl)-1H-benzo [d]imidazol-2-amine.

The invention also relates to analogs of prodrug Compound 6 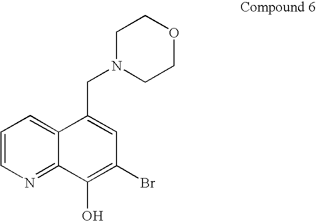
and pharmaceutically acceptable salts, hydrates, and solvates thereof,

wherein any one or more of —H, —Br, —CH₂-morpholino, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 7-bromo-5-(morpholinomethyl)quinolin-8-ol.

The invention also relates to analogs of prodrug Compound 7 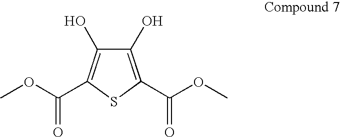
and pharmaceutically acceptable salts, hydrates, and solvates thereof,

wherein any one or more of —H, —OH, —C(O)OCH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; the thiophenyl —S— can be substituted with —O—, and wherein the analog is not dimethyl 3,4-dihydroxythiophene-2,5-dicarboxylate.

The invention also relates to analogs of prodrug Compound 8 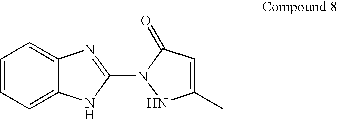
and pharmaceutically acceptable salts, hydrates, and solvates thereof,

wherein any one or more of —H, and —CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 2-(1H-benzo[d]imidazol-2-yl)-5-methyl-1H-pyrazol-3(2H)-one.

The invention also relates to analogs of prodrug Compound 9 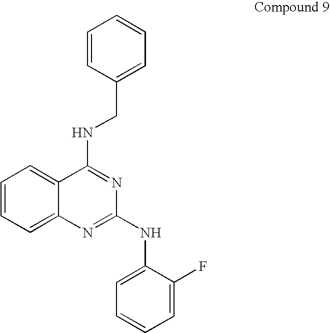
and pharmaceutically acceptable salts, hydrates, and solvates thereof,

wherein any one or more of —H, —F, benzyl, and phenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not N⁴-benzyl-N²-(2-fluorophenyl)quinazoline-2,4-diamine.

The invention also relates to analogs of prodrug Compound 10 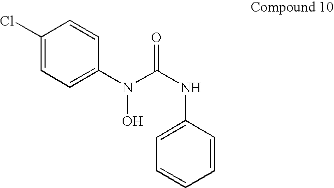
and pharmaceutically acceptable salts, hydrates, and solvates thereof,

wherein any one or more of —H, —OH, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; —C(O) can be substituted with —C(S), and wherein the analog is not 1-(4-chlorophenyl)-1-hydroxy-3-phenylurea.

The invention also relates to analogs of prodrug Compound 11 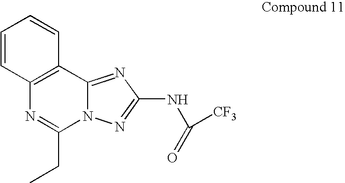
and pharmaceutically acceptable salts, hydrates, and solvates thereof,

wherein any one or more of —H, —F, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not N-(5-ethyl-[1,2,4]triazolo[1,5-c]quinazolin-2-yl)-2,2,2-trifluoroacetamide.

The invention also relates to analogs of prodrug Compound 12 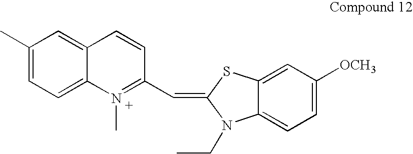
and pharmaceutically acceptable salts, hydrates, and solvates thereof,

wherein any one or more of —H, —CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; the Z configuration can be E, and wherein the analog is not (Z)-3-ethyl-6-methoxy-2-((1,6-dimethylquinolinium-2-yl)methylene)-2,3-dihydrobenzo[d]thiazole.

The invention also relates to analogs of prodrug Compound 13 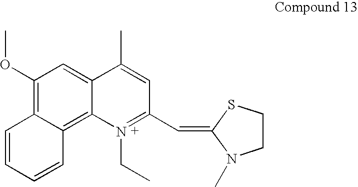
and pharmaceutically acceptable salts, hydrates, and solvates thereof,

wherein any one or more of —H, CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; the Z configuration can be E, and wherein the analog is not (Z)-2-((1-ethyl-6-methoxy-4-methylbenzo[h]quinolinium-2-yl)methylene)-3-methylthiazolidine.

The invention also relates to analogs of prodrug Compound 14 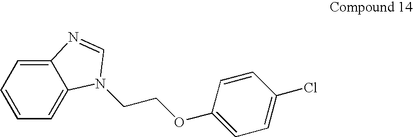
and pharmaceutically acceptable salts, hydrates, and solvates thereof,

wherein any one or more of —H, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 1-[2-(4-chlorophenoxy)ethyl]-1H-benzimidazole.

The invention also relates to analogs of prodrug Compound 15 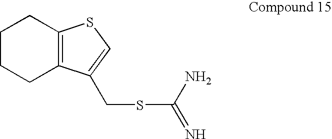
and pharmaceutically acceptable salts, hydrates, and solvates thereof,

wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not (4,5,6,7-tetrahydrobenzo[b]thiophen-3-yl)methyl carbamimidothioate.

In The invention also relates to analogs of prodrug Compound 16 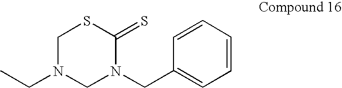
and pharmaceutically acceptable salts, hydrates, and solvates thereof,

wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; and wherein the analog is not 5-ethyltetrahydro-3-(phenylmethyl)-2H-1,3,5-thiadiazine-2-thione.

The invention also relates to analogs of prodrug Compound 17 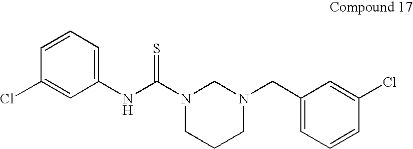
and pharmaceutically acceptable salts, hydrates, and solvates thereof,

wherein any one or more of —H, —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; chlorophenyl can be substituted with heteroaryl, chlorobenzyl can be substituted with alkyl, alkylheteroaryl, and acyl, and wherein the analog is not N-(3-chlorophenyl)-3-[(3-chlorophenyl)methyl]tetrahydro-1(2H)-pyrimidinecarbothioamide.

The invention also relates to analogs of prodrug Compound 18 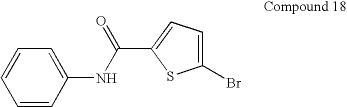
and pharmaceutically acceptable salts, hydrates, and solvates thereof,

wherein any one or more of —H, and —Br, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; —C(O) can be substituted with —C(S), S can be substituted with O, and wherein the analog is not 5-bromo-N-phenyl-2-thiophenecarboxamide.

The invention also relates to analogs of prodrug Compound 19 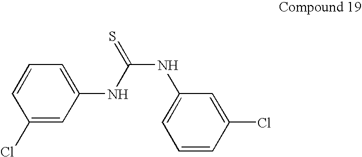
and pharmaceutically acceptable salts, hydrates, and solvates thereof,

wherein any one or more of —H, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; —C(S) can be substituted with —C(O), and wherein the analog is not N,N′-bis(3-chlorophenyl)-thiourea.

Methods for Using the Prodrug Compounds of the Invention

The prodrug compounds of the present invention exhibit the ability to kill bacteria, fungi, protozoa, and helminth and, therefore, can be utilized in order to treat or prevent infections by these organisms. Thus, the Compounds of the Invention are effective in the treatment of any disease or symptom of a disease caused by or resulting from an infection by a bacterium, a protozoan, a fungus, and/or a helminth. In particular, the Compounds of the Invention of the present invention possess excellent cell growth inhibiting effects and are effective in treating upper respiratory tract diseases; infections of catheters; infections of orthopedic prostheses; Urinary Tract Infections (UTI); gastrointestinal infections; heart valves infections; endocarditis; skin infections; chronic wounds; and cystic fibrosis.

Therapeutic Administration

When administered to an animal, the Compounds of the Invention or pharmaceutically acceptable salts of the Compounds of the Invention can be administered neat or as a component of a composition that comprises a physiologically acceptable carrier or vehicle. A composition of the invention can be prepared using a method comprising admixing the Compounds of the Invention or a pharmaceutically acceptable salt of the Compounds of the Invention and a physiologically acceptable carrier, excipient, or diluent. Admixing can be accomplished using methods well known for admixing a Compound of the Invention or a pharmaceutically acceptable salt of the Compound of the Invention and a physiologically acceptable carrier, exipient, or diluent.

The present compositions, comprising Compounds of the Invention or pharmaceutically acceptable salts of the Compounds of the Invention can be administered orally. The Compounds of the Invention or pharmaceutically acceptable salts of Compounds of the Invention can also be administered by any other convenient route, for example, by infusion or bolus injection, by absorption through epithelial or mucocutaneous linings (e.g., oral, rectal, vaginal, and intestinal mucosa) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in release of the Compound of the Invention or a pharmaceutically acceptable salt of the Compound of the Invention into the bloodstream. The mode of administration is left to the discretion of the practitioner.

In one embodiment, the Compound of the Invention or a pharmaceutically acceptable salt of the Compound of the Invention is administered orally.

In another embodiment, the Compound of the Invention or a pharmaceutically acceptable salt of the Compound of the Invention is administered intravenously.

In another embodiment, the Compound of the Invention or a pharmaceutically acceptable salt of the Compound of the Invention can be administered locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, the Compound of the Invention or a pharmaceutically acceptable salt of the Compound of the Invention can be introduced into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the Compound of the Invention or a pharmaceutically acceptable salt of the Compound of the Invention can be formulated as a suppository, with traditional binders and excipients such as triglycerides.

In another embodiment, the Compound of the Invention or a pharmaceutically acceptable salt of the Compound of the Invention can be delivered in a vesicle, in particular a liposome (see Langer, Science 249:1527-1533 (1990) and Treat et al., Liposomes in the Therapy of Infectious Disease and Cancer pp. 317-327 and pp. 353-365 (1989)).

In yet another embodiment, the Compound of the Invention or a pharmaceutically acceptable salt of the Compound of the Invention can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment, a controlled- or sustained-release system can be placed in proximity of a target of the Compound of the Invention or a pharmaceutically acceptable salt of the Compound of the Invention, e.g., the reproductive organs, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment, the physiologically acceptable excipients are sterile when administered to an animal. The physiologically acceptable excipient should be stable under the conditions of manufacture and storage and should be preserved against the contaminating action of microorganisms. Water is a particularly useful excipient when the Compound of the Invention or a pharmaceutically acceptable salt of the Compounds of the Invention is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The Compound of the Invention or pharmaceutically acceptable salt of the Compound of the Invention of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences pp. 1447-1676 (Alfonso R. Gennaro, ed., 19th ed. 1995).

In one embodiment, the Compound of the Invention or a pharmaceutically acceptable salt of the Compound of the Invention is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided Compound of the Invention or pharmaceutically acceptable salt of the Compound of the Invention. In tablets, the Compound of the Invention or pharmaceutically acceptable salt of the Compound of the Invention is mixed with a carrier having the necessary compression properties in suitable proportions and compacted in the shape and size desired. The powders and tablets can contain up to about 99% of the Compound of the Invention or pharmaceutically acceptable salt of the Compound of the Invention.

Capsules may contain mixtures of the Compounds of the Invention or pharmaceutically acceptable salts of the Compounds of the Invention with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the Compound of the Invention or a pharmaceutically acceptable salt of the Compound of the Invention can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the Compound of the Invention or a pharmaceutically acceptable salt of the Compound of the Invention is to be administered by infusion, it can be dispensed, for example, with an infusion bottle containing sterile pharmaceutical grade water or saline. Where the Compound of the Invention or a pharmaceutically acceptable salt of the Compound of the Invention is administered by injection, an ampule of sterile water for injection or saline can be provided so that the ingredients can be mixed prior to administration.

In another embodiment, the Compound of the Invention or pharmaceutically acceptable salt of the Compound of the Invention can be administered transdermally through the use of a transdermal patch. Transdermal administrations include administrations across the surface of the body and the inner linings of the bodily passages including epithelial and mucosal tissues. Such administrations can be carried out using the present Compounds of the Invention or pharmaceutically acceptable salts of the Compounds of the Invention, in lotions, creams, foams, patches, suspensions, solutions, and suppositories (e.g., rectal or vaginal).

Transdermal administration can be accomplished through the use of a transdermal patch containing the Compound of the Invention or pharmaceutically acceptable salt of the Compound of the Invention and a carrier that is inert to the Compound of the Invention or pharmaceutically acceptable salt of the Compound of the Invention, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the Compound of the Invention or pharmaceutically acceptable salt of the Compound of the Invention into the blood stream, such as a semi-permeable membrane covering a reservoir containing the Compound of the Invention or pharmaceutically acceptable salt of the Compound of the Invention with or without a carrier, or a matrix containing the active ingredient.

The Compounds of the Invention or pharmaceutically acceptable salts of the Compounds of the Invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The Compound of the Invention or a pharmaceutically acceptable salt of the Compound of the Invention can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the Compound of the Invention or a pharmaceutically acceptable salt of the Compound of the Invention, and can thus reduce the occurrence of adverse side effects.

Controlled- or sustained-release compositions can initially release an amount of the Compound of the Invention or a pharmaceutically acceptable salt of the Compound of the Invention that promptly produces the desired therapeutic or prophylactic effect, and gradually and continually release other amounts of the Compound of the Invention or a pharmaceutically acceptable salt of the Compound of the Invention to maintain this level of therapeutic or prophylactic effect over an extended period of time. To maintain a constant level of the Compound of the Invention or a pharmaceutically acceptable salt of the Compound of the Invention in the body, the Compound of the Invention or a pharmaceutically acceptable salt of the Compound of the Invention can be released from the dosage form at a rate that will replace the amount of the Compound of the Invention or a pharmaceutically acceptable salt of the Compound of the Invention being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions.

In certain embodiments, the present invention is directed to prodrugs of the Compounds of the Invention or pharmaceutically acceptable salts of Compounds of the Invention of the present invention. Various forms of prodrugs are known in the art, for example as discussed in Bundgaard (ed.), Design of prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Deilvery Systems, American Chemical Society (1975).

The amount of the Compound of the Invention or a pharmaceutically acceptable salt of the Compound of the Invention that is effective for treating an infection can be determined using standard clinical techniques. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. The typical dose will range from will typically range from about 0.001 mg/kg to about 250 mg/kg of body weight per day, in one embodiment, from about 1 mg/kg to about 250 mg/kg body weight per day, in another embodiment, from about 1 mg/kg to about 50 mg/kg body weight per day, and in another embodiment, from about 1 mg/kg to about 20 mg/kg of body weight per day. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one Compound of the Invention or a pharmaceutically acceptable salt of the Compound of the Invention is administered, the effective dosage amounts correspond to the total amount administered.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, pre-filled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The Compound of the Invention or a pharmaceutically acceptable salt of the Compound of the Invention can be assayed in vitro or in vivo for the desired therapeutic or prophylactic activity prior to use in humans. Animal model systems can be used to demonstrate safety and efficacy.

In another embodiment, the pharmaceutically acceptable carrier is suitable for oral administration and the composition comprises an oral dosage form.

The Compounds of the Invention and pharmaceutically acceptable salts of Compounds of the Invention can be prepared using a variety of methods starting from commercially available compounds, known compounds, or compounds prepared by known methods.

Compounds 1-19 are commercially available from TimTec Inc. (Newark, Del.), ChemDiv, Inc. (San Diego Calif.), Life Chemicals Inc. (formerly I.F. Lab Ltd. Burlington, Canada) and Maybridge, plc, (Cornwall, UK) and therefore their syntheses are known. Those of skill in the art employing known organic chemical synthetic methods can synthesize any of the Compounds of the Invention. For example, treatment of an alkyl or aryl group with lithium, followd by reaction with an electrophile, will substitute an —H with C₁₋₆ alkyl, C₂₋₆ alkenyl, C₂₋₆ alkynyl, —C(O)C₁₋₆alkyl, —C(O)OC₁₋₆alkyl, C₃₋₆ cycloalkyl, C₃₋₆ cycloalkyl-C₁₋₃ alkyl, alkylaryl, aryl, arylalkyl, heteroaryl, or heteroarylalkyl (see e.g. Carey F. A., and Sundberg, R. J., Advanced Organic Chemistry, 4^{th} ed., Plenum Publishers, Boston, pp. 444, 693, 714, 885 (2001). Reaction of aryl groups with strong acids such a HNO₃, HCN, or HCl will substitute an H for NO₂, CN, or Cl, respectively (See Carey and Sundberg p. 191). Further acid hydrolysis of the CN moiety will liberate a carboxylic acid moiety that can be further derivatized by esterification, reduction or amination. (see Patrick, G. Organic Chemistry, Springer Verlag, N.Y., p. 220-221 (2000) (Patrick). Alkylation and acylation of amines will change the substitution on the nitrogens of the prodrug compounds (see e.g. Patrick, p. 299).

### EXAMPLES

### Example 1

### Pilot Screens with Mutant Pools

Pilot screens were performed to identify compounds that have a lower activity against a bacterial strain deleted in an activating enzyme as compared to the wild type.

A complete, ordered E. coli K12 knockout library of 4320 genes and predicted ORFs (the Keio library) was used (Baba, et al. (2006). Mol. Systems Biol. 2:2006.0008). The knockouts were constructed using the Wanner method with a kanamycin cassette replacing the ORFs (Datsenko et al. (2000) Proc. Natl. Acad. Sci. USA 97:6640-6645). All strains of this library were combined in a mix (BacPool 1) for screening. This permitted screening of the library against all strains simultaneously, instead of screening a full industry-size library against each of the individual E. coli 4×10³ knockout strains.

The library mix was prepared by first culturing all strains overnight in LB medium, and then adding equal aliquots into a tube. This material was then mixed, dispensed in vials and stored at −80° C. For the screen, one vial was thawed, diluted to 10⁵ cells/ml in LB, and dispensed into 384 well microtiter plates. The compounds were added from DMSO stocks at a final concentration of 46 μg/ml. The same compounds were added to plates containing the wild type isogenic control. Screening was performed at the Harvard NSRB/NERCE screening facility that host a collection of 150,000 compounds (see, Table 2). All screens at NSRBINERCE were performed in duplicate, which strongly decreases the rate of false positives and negatives. For each tested molecule there are three possible scenarios when scoring for growth/no growth:

Growth in both the BacPool and K12 wells: indicates lack of antimicrobial activity;

Growth inhibition in both the BacPool and K12 wells: a possible antibiotic (direct activity) or generally toxic compound is present, but is not a prodrug; or

Growth of a BacPool well and no growth of the K12 well: a prodrug hit.

After an overnight incubation of plates at 37° C., the plates were read at OD600. Compounds that had no effect on the growth of the mix and inhibited the growth of the wild type were recorded as hits. The wells with growth indicated a prodrug hit that is not converted into a drug by a particular deletion mutant in the mix. The mutant strain is then identified in a secondary screen (see, Example 2).

TABLE 2
The NSRB/NERCE library.

| | Number of | ICCB Plate |
|---|---|---|
| Library Name | Compounds | Numbers |
| Biomol-TimTec 1 | 8,518 | 1534-1558 |
| Bionet 1 | 4,800 | 0568-0582 |
| Bionet 2 | 1,700 | 1364-1368 |
| CEREP | 4,800 | 0526-0540 |
| *Chem Bridge DiverSet E | 16,320 | 0001-0051 |
| ChemBridge Microformat | 50,000 | 0686-0842 |
| ChemDiv1 (Combilab and | 28,864 | 0587-0668 |
| International) | | |
| ChemDiv 2 | 8,560 | 1369-1393 |
| ChemDiv 3 | 16,544 | 1473-1519 |
| ChemDiv-Anti-Mitotic | 1,254 | 1157-1160 |
| Collection | | |
| *Commercial Diversity | 5,056 | 1231-1245 |
| Set 1 | | |
| Enamine 1 | 6,004 | 1394-1411 |
| I.F. Lab 1 | 6,543 | 1412-1430 |
| I.F. Lab 2 | 292 | 1459 |
| Maybridge 1 | 8,800 | 0542-0566 |
| Maybridge 2 | 704 | 1303-1304 |
| Maybridge 3 | 7,639 | 1431-1452 |
| Maybridge 4 | 4,576 | 1521-1533 |
| Peakdale 1 | 2,816 | 0518-0525 |
| Peakdale 2 | 352 | 1305 |
| Mixed Commercial Plate 1 | 352 | 0541 |
| Mixed Commercial Plate 2 | 320 | 0567 |
| Mixed Commercial Plate 3 | 251 | 0669 |
| Mixed Commercial Plate 4 | 331 | 1306 |
| Mixed Commercial Plate 5 | 268 | 1520 |

Known Bioactives Collections

| | | |
|---|---|---|
| NINDS Custom Collection | 1,040 | 0500-0503 |
| *http://iccb.med.harvard.edu/screening/compound_libraries/nci_open_collection.htmICCB | 489 | 0684-0685 |
| Bioactives 1 | | |
| SpecPlus Collection | 960 | 1081-1083 |
| BIOMOL ICCB Known Bioactives | 480 | 1361-1362 |

*currently not available for screening

The general procedure to establish the Z′-factor was tested. Since the output of the screening is a typical growth/no growth assay, this was performed by comparing growth in control wells to those containing a model antimicrobial, ciprofloxacin at 30 μg/ml. E. coli K12 were cultured in LB medium, and exponentially-growing cells were dispensed at 10⁶ /ml in 384-well microtiter plates, 30 μl/well. Six plates were used in this experiment. Ciprofioxacin was added to half of the wells (3 μl in LB, bringing the final volume to 33 μl). After an overnight incubation at 37 C, the OD₆₀₀ of the plates were read, and the values of each well were used to calculate Z′-factor: Z′=1−(3SD++3SD−)/IAve₊−Ave.I, where: SD+=positive control standard deviation; SD−=negative control standard deviation; Ave+=positive control average; and Ave−=negative control average). The following table for Z′-factor interpretation was used:

High-throughput Screening Assay Fitness:

1>Z′>0.9 An excellent assay;

0.9>Z′>0.7 A good assay;

0.7>Z′>0.5 Hit selection will benefit significantly from any improvement; and

0.5=Z′ The absolute minimum recommend for high throughput screening

The Z′-factor for the assay was 0.75, suggesting that pilot screening could be performed. It is important to note that the deviation from perfect results in this screen was due to normal variations in OD reading among wells, rather than to false positives or false negatives. There was no case of substantial growth in a well with ciprofloxacin or lack of growth in a well without an antibiotic.

A first pilot screen of 3000 compounds from the NERCE library was performed in duplicate to reduce variability (screening in duplicate is standard procedure at this facility). The controls were E. coli W3100 cells, which were compared to a pool of 4320 knockout strains from the Keio library (BacPool). The pool was prepared by growing each mutant overnight in microtiter plates in LB at 37° C. and then mixing all of them in equal amounts. The compounds were dispensed at a final concentration of 46 μg/ml in 275 nl volume. The screen produced 3 hits.

In a second pilot screen of 45,000 compounds from the NERCE library were tested and 17 prodrug hits were obtained (i.e., a frequency of 0.037%).

### Example 2

### Secondary Screen

Once the hits are obtained from the screen, they can be verified by retesting the hit compounds against the mix and the wild type. Confirmed hits are examined further. Strains containing the activating enzymes are determined with a secondary screen against individual deletion strains.

In the secondary screen, the deletion strain lacking the activating enzyme is identified by testing against the strains of the knockout library dispensed in individual wells. This will identify the resistant strain lacking an activating enzyme.

### Example 3

### Hit Validation Using Strains Overexpressing Prodrug Activating Enzymes

Next, the hits that verify and have reduced activity compared to wild type or no activity against strains deleted in a known or putative enzyme are validated. The rationale is to test the hit against a strain overexpressing the putative activating enzyme. Strains overexpressing activating enzymes have considerably higher susceptibility to prodrugs. It is important to note that conventional antibiotics that inhibit specific targets have increased activity against strains with diminished expression of the target, and decreased activity if the target is overproduced (Schmid, M. B. (2001) New targets and strategies for identification of novel classes of antibiotics. In Antibiotic Development and Resistance. Hughes, D. and Andersson, D. I. (eds). New York: Taylor and Francis, pp. 197-208; Sun, D.e.a. (2001) In 41st Interscience Conference on Antimicrobial Agents and Chemotherapy Chicago: ASM, pp. 77). A prodrug hit has higher activity against a strain overexpressing an activating enzyme, and lower activity against a strain lacking/diminished in an activating enzyme. This behavior of a prodrug hit is the exact opposite from what one expects from a specific antibiotic, and will provide for an excellent preliminary validation of prodrugs. This validation is facilitated by the availability of the ASKA library of E. coli strains overexpressing all known ORFs. The University of Nagoya freely distributes this resource (Saka et al. (2005) DNA Res. 12:63-68). The library contains 4,382 individual E. coli K12 W3110 clones, each carrying a single ORF cloned in an expression vector pCA24N under a pT5/lac promoter. The N-termini carry a his-tag linked to the ORF by a 7 amino acid spacer. The vector carries a CAM resistance marker and a lacl^{q} gene for a tight control of IPTG-inducible expression.

The ASKA strains of interest are validated for functional expression of the enzyme. If the protein is not expressed from a given expression vector the ORF will be recloned. A strain overexpressing the activating enzyme from the ASKA library is then tested with the hit compound. If the hit has greater activity against this overexpressing strain as compared to the wild type, this indicates a prodrug. The test is performed in a standard broth microdilution assay for MIC determination. Hits with the lowest wild type MIC are then focused on.

### Example 4

### Validation of Deletion and Oveexpression of Prodrug Activating Enzyme Screens

Known antimicrobial prodrugs were used in order to validate the proposed screen with E. coli strains overexpressing the activating enzymes. Most known prodrugs are specific for M. tuberculosis, and the broader-spectrum metronidazole is ineffective against E. coli. Metronidazole was reexamined because its activity may be within a measurable range with an E. coli strain overexpressing an activating enzyme. A number of E. coli activating enzymes that have been developed to activate prodrugs used in cancer chemotherapy were also utilized (Table 3). The approach, known as Gene-Directed Enzyme-Prodrug Therapy (GDEPT), is based on delivering a gene coding for the bacterial activating enzyme into cancer tissue, and then adding a prodrug that converts into a cytotoxic compound in the cell. Dinitroaziridinylbenzamide and dinitrobenzamide convert into active antiseptic-like molecules, and in this regard match the type of compounds that are sought in the screen. Fludarabine and 5-fluorocytosine are analogs of nucleotides that stop cells from growing when incorporated into DNA. Whether these compounds are active against bacteria is unknown.

TABLE 3
Prodrugs used in Cancer Gene-Directed Enzyme-Prodrug Therapy.

| Molecule | Structure | Activating enzyme |
|---|---|---|
| 5-Fluorocytosine | 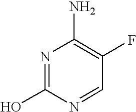 | Cytosine deaminase (CodA)(Mullen et al., 1992; Tiraby et al., 1998) |
| Fludarabine | 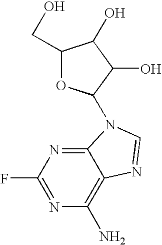 | Purine nucleoside phosphorylase (DeoD)(Huang and Plunkett, 1987) |
| Dinitroaziridinylbenzamide (CB1954) | 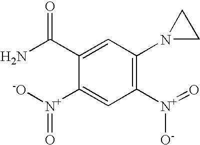 | Nitroreductase (NfnB/NfsA)(Knox et al., 1988; Knox et al., 1992) |
| 3,5-Dinitrobenzamide | 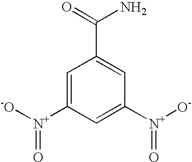 | Nitroreductase (NfnB/NfsA)(Denny, 2003) |

The prodrugs are converted into drugs by the cancer cells expressing the corresponding bacterial enzyme.

Metronidazole is converted into an active drug by the nitrate reductase of H. pylon (van der Wouden et al., 2001) and other bacteria. E. coli has two nitrate reductases −NfnB and NfsA. As expected, metronidazole was essentially ineffective against the wild type, with an MIC >500 μg/ml (Table 4). However, metronidazole appeared to be an effective antimicrobial against the strain overexpressing NfnB and NfsA (MIC 8.8 μg/ml). Importantly, strains deleted in the enzymes showed even greater resistance than the wild type. This confirms our proposed use of deletion strains to validate prodrug candidates. Indeed, opposing susceptibilities of an overexpressing versus a deleted strain strongly points to the prodrug nature of a hit compound.

Similarly, significantly increased susceptibilities were observed with dinitroaziridinylbenzamide and dinitrobenzamide against strains overexpressing the corresponding activating enzymes. 5-fluorocytosine showed little activity against any strains tested. Taken together, the results suggest that differential expression of an activation enzyme can be used to develop a specific screen for prodrugs.

TABLE 4
Effect of Overexpression and Deletion of an Activating Enzyme

| | | E. coli K12 | | | |
|---|---|---|---|---|---|
| | | overexpressing | | E. coli K12 | |
| | E. coli | activating | | Δactivating | |
| Compound | K12 wt | enzyme | | enzyme | |
| Metronidazole | 563 | nfnB⁺ | 8.8 | nfnB⁻ | 1125 |
| | | nfsA⁺ | 8.8 | nfsA⁻ | 2250 |
| Dinitroaziridinylbenzamide | >200 | nfnB⁺ | 3.3 | nfnB⁻ | >200 |
| (CB1954) | | nfsA⁺ | 6.3 | nfsA⁻ | >200 |
| 3,5-Dinitrobenzamide | 250 | nfnB⁺ | 15.6 | nfnB⁻ | >500 |
| | | nfsA⁺ | 7.8 | nfsA⁻ | >500 |
| Fludarabine | >500 | deoD⁺ | 125 | deoD⁻ | >500 |
| 5-fluorocytosine | >2500 | codA⁺ | 625 | codA⁻ | >2500 |
| Erythromycin | 200 | nfnB⁺ | 200 | | |
| | | nfsA⁺ | 200 | | |
| | | deoD⁺ | 200 | | |
| | | codA⁺ | 200 | | |

### Example 5

### Prodrug Screening Based on Essential Protein Overexpression

A different modality of the prodrug screen is examined based on the increased sensitivity to prodrugs of a strain overexpressing an activating enzyme as compared to the wild type. For this screen, strains overexpressing enzymes from the ASKA library described above are used. Since each strain has to be screened individually, the number of strains is limited to those that express enzymes that are essential and conserved.

There are approximately 300 essential genes in E. coli (Gerdes et al. (2003) J. Bacteriol. 185:5673-5684), and from this list essential known and putative enzymes were identified (Table 5). Apart from the annotation of an essential protein as an enzyme, two additional significant criteria are used—absence of an obvious homolog in humans; and presence of a homolog in M. tuberculosis. Lack of human homologs increases the chances of finding non-toxic compounds. Conservation among E. coli and M. tuberculosis indicates a generally conserved nature of potential prodrug-activating enzymes, and treatment of tuberculosis is one of the important applications for sterilizing antimicrobial compounds. Using these criteria, a select set of 50 potential prodrug-activating enzymes is obtained.

TABLE 5
Essential Candidate Prodrug Activating Genes.

| | | Mtub | Human | | | | |
|---|---|---|---|---|---|---|---|
| Gene | Essential | homolog | homolog | Length | SwissProt | B-name | Annotation |
| AckA | Y | Y | N | 400 | P15046 | b2296 | Acetate kinase (EC |
| | | | | | | | 2.7.2.1) |
| ArgC | Y | Y | N | 334 | P11446 | b3958 | N-acetyl-gamma- |
| | | | | | | | glutamyl-phosphate |
| | | | | | | | reductase (EC |
| | | | | | | | 1.2.1.38) |
| asd | Y | Y | N | 367 | P00353 | b3433 | Aspartate- |
| | | | | | | | semialdehyde |
| | | | | | | | dehydrogenase (EC |
| | | | | | | | 1.2.1.11) |
| BtuR | Y | Y | N | 196 | P13040 | b1270 | COB(I)alamin |
| | | | | | | | adenosyltransferase |
| | | | | | | | (EC 2.5.1.17) |
| CoaD | Y | Y | N | 159 | P23875 | b3634 | Phosphopantetheine |
| | | | | | | | adenylyltransferase |
| | | | | | | | (EC 2.7.7.3) |
| CysE | Y | Y | N | 273 | P05796 | b3607 | Serine |
| | | | | | | | acetyltransferase (EC |
| | | | | | | | 2.3.1.30) |
| DapA | Y | Y | Y/N | 292 | P05640 | b2478 | Dihydrodipicolinate |
| | | | | | | | synthase (EC |
| | | | | | | | 4.2.1.52) |
| DapB | Y | Y | N | 273 | P04036 | b0031 | Dihydrodipicolinate |
| | | | | | | | reductase (EC |
| | | | | | | | 1.3.1.26) |
| DapD | Y | Y | N | 274 | P03948 | b0166 | Tetrahydrodipicolinate |
| | | | | | | | N- |
| | | | | | | | succinyltransferase |
| | | | | | | | (EC 2.3.1.117) |
| DapF | Y | Y | N | 275 | P08885 | b3809 | Diaminopimelate |
| | | | | | | | epimerase (EC |
| | | | | | | | 5.1.1.7) |
| DdlB | Y | Y | N | 306 | P07862 | b0092 | D-alanine--D-alanine |
| | | | | | | | ligase B (EC 6.3.2.4) |
| Dxr | Y | Y | N | 398 | P45568 | b0173 | 1-deoxy-D-xylulose |
| | | | | | | | 5-phosphate |
| | | | | | | | reductoisomerase (EC |
| | | | | | | | 1.1.1.267) |
| ElaA | Y | Y | N | 153 | P52077 | b2267 | GTP-binding protein |
| | | | | | | | ElaA |
| FbaA | Y | Y | N | 359 | P11604 | b2925 | Fructose- |
| | | | | | | | bisphosphate aldolase |
| | | | | | | | class II (EC 4.1.2.13) |
| FrlD | Y | Y | N | 261 | P45543 | b3374 | Fructoselysine kinase |
| FtsI | Y | Y | N | 588 | P04286 | b0084 | Peptidoglycan |
| | | | | | | | synthetase |
| HemD | Y | Y | N | 246 | P09126 | b3804 | Uroporphyrinogen-III |
| | | | | | | | synthase (EC |
| | | | | | | | 4.2.1.75) |
| IspE | Y | Y | N | 283 | P24209 | b1208 | 4-diphosphocytidyl-2- |
| | | | | | | | C-methyl-D-erythritol |
| | | | | | | | kinase (EC 2.7.1.148) |
| IspF | Y | Y | N | 159 | P36663 | b2746 | 2-C-methyl-D- |
| | | | | | | | erythritol 2,4- |
| | | | | | | | cyclodiphosphate |
| | | | | | | | synthase (EC |
| | | | | | | | 4.6.1.12) |
| IspG | Y | Y | N | 372 | P27433 | b2515 | 1-hydroxy-2-methyl- |
| | | | | | | | 2-(E)-butenyl 4- |
| | | | | | | | diphosphate synthase |
| | | | | | | | (gcpE) |
| IspH | Y | Y | N | 316 | P22565 | b0029 | 4-hydroxy-3- |
| | | | | | | | methylbut-2-enyl |
| | | | | | | | diphosphate reductase |
| Lgt | Y | Y | N | 291 | P37149 | b2828 | Prolipoprotein |
| | | | | | | | diacylglyceryl |
| | | | | | | | transferase (EC |
| | | | | | | | 2.4.99.—) |
| MenE | Y | Y | Y/N | 451 | P37353 | b2260 | O-succinylbenzoic |
| | | | | | | | acid--CoA ligase (EC |
| | | | | | | | 6.2.1.26) |
| MesJ | Y | Y | N | 432 | P52097 | b0188 | tRNA(Ile)-lysidine |
| | | | | | | | synthetase |
| MrdA | Y | Y | N | 633 | P08150 | b0635 | Penicillin-binding |
| | | | | | | | protein 2, |
| | | | | | | | transglycosylase/trans |
| | | | | | | | peptidase |
| Mtn | Y | Y | N | 232 | P24247 | b0159 | MTA/SAH |
| | | | | | | | nucleosidase |
| MurC | Y | Y | N | 491 | P17952 | b0091 | UDP-N- |
| | | | | | | | acetylmuramate-- |
| | | | | | | | alanine ligase (EC |
| | | | | | | | 6.3.2.8) |
| MurD | Y | Y | N | 438 | P14900 | b0088 | UDP-N- |
| | | | | | | | acetylmuramoylalanine-- |
| | | | | | | | D-glutamate ligase |
| | | | | | | | (EC 6.3.2.9) |
| murE | Y | Y | N | 495 | P22188 | b0085 | UDP-N- |
| | | | | | | | acetylmuramoylalanyl- |
| | | | | | | | D-glutamate--2,6- |
| | | | | | | | diaminopimelate |
| | | | | | | | ligase (EC 6.3.2.13) |
| MurG | Y | Y | N | 355 | P17443 | b0090 | UDP-N- |
| | | | | | | | acetylglucosamine-- |
| | | | | | | | N-acetylmuramyl- |
| | | | | | | | (Pentapeptide) |
| | | | | | | | pyrophosphoryl- |
| | | | | | | | undecaprenol N- |
| | | | | | | | acetylglucosamine |
| | | | | | | | transferase (EC |
| | | | | | | | 2.4.1.—) |
| MurI | Y | Y | N | 289 | P22634 | b3967 | Glutamate racemase |
| | | | | | | | (EC 5.1.1.3) |
| Pay | Y | Y | N | 196 | P77181 | b1400 | Phenylacetic acid |
| | | | | | | | degradation protein, |
| | | | | | | | predicted |
| | | | | | | | acyltansferase |
| PspE | Y | Y | N | 104 | P23857 | b1308 | Rhodanese-related |
| | | | | | | | sulfurtransferase |
| PyrH | Y | Y | N | 241 | P29464 | b0171 | Uridylate kinase (EC |
| | | | | | | | 2.7.4.—) |
| Rib | Y | Y | N | 217 | P24199 | b3041 | 3,4-dihydroxy-2- |
| | | | | | | | butanone 4-phosphate |
| | | | | | | | synthase |
| RibD | Y | Y | N | 367 | P25539 | b0414 | Riboflavin-specific |
| | | | | | | | deaminase/HTP |
| | | | | | | | reductase (EC |
| | | | | | | | 3.5.4.26/EC |
| | | | | | | | 1.1.1.193) |
| TdcG | Y | Y | N | 140 | P42630 | b3112 | L-serine dehydratase |
| | | | | | | | (EC 4.2.1.13) |
| ThiL | Y | Y | N | 325 | P77785 | b0417 | Thiamine- |
| | | | | | | | monophosphate |
| | | | | | | | kinase (EC 2.7.4.16) |
| YagS | Y | Y | N | 318 | P77324 | b0285 | Putative xanthine |
| | | | | | | | dehydrogenase yagS, |
| | | | | | | | FAD binding subunit |
| | | | | | | | (EC 1.1.1.204) |
| YahF | Y | Y | N | 515 | P77187 | b0320 | Predicted acyl-CoA |
| | | | | | | | synthetase subunit |
| YbeY | Y | Y | N | 155 | P77385 | b0659 | Predicted metal- |
| | | | | | | | dependent hydrolase |
| YbhA | Y | Y | N | 272 | P21829 | b0766 | Predicted HAD |
| | | | | | | | family hydrolase |
| YcdX | Y | Y | N | 245 | P75914 | b1034 | Predicted PHP family |
| | | | | | | | hydrolase |
| YciL | Y | Y | N | 291 | P37765 | b1269 | Predicted RluB-like |
| | | | | | | | pseudouridylate |
| | | | | | | | synthase |
| YdjQ | Y | Y | N | 295 | P76213 | b1741 | Predicted nuclease |
| YeaZ | Y | Y | N | 231 | P76256 | b1807 | Predicted protease |
| YfcH | Y | Y | N | 297 | P77775 | b2304 | Predicted nucleoside- |
| | | | | | | | diphosphate sugar |
| | | | | | | | epimerase |
| yfgE | Y | Y | N | 248 | P76570 | b2496 | DnaA paralog, |
| | | | | | | | predicted DNA |
| | | | | | | | replication initiation |
| | | | | | | | ATPase |
| YjeE | Y | Y | N | 153 | P31805 | b4168 | Predicted |
| | | | | | | | ATP/GTPase |

Strains in the ASKA library have not been functionally validated. A straightforward genetic approach is used to validate the functionality of the recombinant enzymes. The rationale is to create a disruption in the chromosomal copy of the cognate gene in the presence of IPTG. The disruptions are made by the Wanner method, which replaces an ORF with a kan resistance cassette. In parallel, disruptions are made in the wild type control. The disruption is verified by PCR amplification of the expected flanking region and verify its size. The ability to make a disruption in the ASKA strain expressing a recombinant enzyme, but not in the wild type control, indicates functional expression of the protein.

This set of 50 enzymes is smaller than the full set of strains carrying in vitro non-essential enzymes. Therefore, it is screened with a large, industry-size compound library to increase the probability of obtaining hits (e.g., a 500,000 compound library). There is no need to screen the entire library against strains differentially expressing an activating enzyme. Prodrugs are found only among compounds that have antimicrobial activity. Therefore, the 500,000 compound library is first screened against wild type E. coli W3100, and the hits are reformatted into an active sublibrary.

In order to arrive at a realistic number of operations, the equivalent of 2 full library screens are performed. (or 10⁶). The size of the active sublibrary is then 10⁶/50=20,000 compounds. A pilot screen is run with ˜10,000 compounds of the original library to determine the hit rate at several concentrations (5, 10, 20 and 40 μg/ml), and then one can choose the one that produces an ˜4% hit rate that will result in a 20,000 compound active sub-library. In order to differentiate between the control strain and one overexpressing an activating enzyme, the test compounds are applied at a concentration less than used to identify compounds active against the control E. coli. Therefore, the active sub-library of 20,000 compounds are retested against the wild type at 4 additional concentrations −10, 5, 2.5, and 1 μg/ml. In this manner, the approximate minimal active concentration for each compound is established. For testing prodrug candidates with strains overexpressing potential activating enzymes, compounds are tested at ⅕ minimal concentration obtained with the isogenic control strain. For this, compounds are grouped according to the concentration at which they are tested, in order to permit a uniform delivery of library compounds with pin dispensers.

Compounds that show activity against strains overexpressing the activating enzyme but not the wild type at the same test concentration are hits. The hits obtained are verified by retesting their activity against the overexpression and wild type strain. Verified hits are then validated by testing their activity against strains with diminished expression of the essential activating enzyme. Such strains are constructed using an antisense approach. High activity against the strain overexpressing the enzyme and low activity against a strain with diminished expression points to a candidate prodrug. Further validation includes measuring the MIC, MBC, sterilization activity against stationary cells and ability to avoid TolC-dependent MDR efflux.

### Example 6

### Screening for Compounds Having Reduced Mutltidrug Pump Efflux

An ability of a prodrug to avoid MDR efflux is an additional indicator of the prodrug mode of action, and a predictor of a broad action spectrum. A simple test is used to ascertain this property of the prodrug hits. The rationale is to test the effect of a tolC mutation on drug susceptibility. TolC is an outer membrane porin used by the major E. coli transenvelope MDRs such as AcrAB for docking, and tolC mutants are very sensitive to antibiotics. A tolC::cam mutation is moved from an E. coli K12 tolC::cam into a strain deleted in the activating enzyme and the wild type, selecting for chloramphenicol resistance. The overexpression strain carries cam resistance of the plasmid. Therefore, in order to construct an overexpression strain deleted in tolC, a tolC::kan disruption cassette is moved from E. coli W3110 tolC::kan into it by P1 transduction. Comparable activity of each strain +/− the tolC mutation strongly indicates the insensitivity to efflux. Note that such a result does not prove that the compound will be impervious to efflux in some other organisms. However, having a number of compounds that can bypass MDR efflux in E. coli will indicate that the screen is useful for discovering a broad-spectrum antiinfective.

### Example 7

### Inhibition of Multidrug Pump Efflux

Prodrugs are activated by activating enzymes into reactive molecules that bind to their targets, creating an irreversible sink. This may lead to insensitivity of the overall process to MDR efflux. In order to test this possibility, an E. coli K12 with a deletion in tolC coding for the outer membrane porin that is a component of the transenvelope MDRs was utilized (Li, X. Z et al. (2004) Drugs 64:159-204). This strain is highly sensitive to antibiotics (Tegos et al. (2002) Antimicrob. Agents Chemother. 46:3133-3141). Among the several E. coli MDRs that dock to TolC, AcrAB is significantly expressed and is primarily responsible for the intrinsic resistance of this bacterium to antibiotics. The substrate specificity of AcrAB is remarkably broad, and includes essentially all small molecular weight amphipathic compounds. The AcrAB substrates include anions (SDS, fatty acids, bile acids), neutral compounds (macrolides, chloramphenicol, tetracyclines) and cations or compounds that can form cations (acridine, quaternary compound antiseptics, fluoroquinolones). AcrAB can extrude the best natural broad spectrum antibiotics that evolved for good penetration —chloramphenicol and tetracycline; and extrude the best synthetic broad-spectrums, the fluoroquinolones. Indeed, amphipathic antimicrobials that are not an AcrAB substrate are not known. The reason compounds like fluoroquinolones or tetracycline are active against E. coli is that they are used at levels that can overwhelm this and other MDRs. Knocking out tolC increases susceptibility at least 4 fold for the best penetrating compounds, and up to 1,000 fold for those that are good substrates of the pump (Tegos et al. (2002) Antimicrob. Agents Chemother. 46:3133-3141).

Cells were tested in a standard broth microdilution assay, inoculating 10⁵ cells per well of a microtiter plate in LB broth. Overexpression and deletion strains were compared to the appropriate isogenic wild type. IPTG was added at 1 mM to growth medium. Experiments involving nfnB and nfsA strains were performed under anaerobic conditions.

Amphipathic cations are useful substrates for all classes of MDRs, including the RND AcrAB pump (Hsieh et al. (1998) Proc. Natl. Acad. Sci. USA 95:6602-6606; Stermitz et al. (2000a) Proc. Natl. Acad. Sci. USA 97:1433-1437; Lewis, K. (2001b) J. Mol. Microbiol. Biotechnol. 3:247-254; Tegos et al. (2002) Antimicrob. Agents Chemother. 46:3133-3141). The prodrugs that have been tested, metronidazole, dinitroaziridinylbenzamide and dinitrobenzamide, are all amphipathic cations and should be effectively extruded from E. coli.

Remarkably, there was no effect of tolC deletion on susceptibility of E. coli to prodrugs (Table 6). A control with erythromycin shows the typical decrease in MIC from 200 μg/ml in the wild type to 1.56 μg/ml in the ΔtolC strain, a 128 fold change. This observation suggests that prodrugs may counter efflux by activating into products that become trapped inside the cell by covalent attachment to their targets.

TABLE 6
The MIC (in μg/ml) of Prodrugs with E. coli Strains
Overexpressing and Deleted in the Activating Enzyme.

| | Strain | | | | | | |
|---|---|---|---|---|---|---|---|
| | | E. coli K12 | | | | | |
| | | ΔtolC | | E. coli K12 | | | |
| | | overexpressing | | overexpressing | | E. coli K12 | |
| | E. coli | activating | | activating | | Δactivating | |
| Compound | K12 wt | enzyme | | enzyme | | enzyme | |
| Metronidazole | 563 | nfnB⁺ | 8.8 | nfnB⁺ | 8.8 | nfnB⁻ | 1125 |
| | | nfsA⁺ | 8.8 | nfsA⁺ | 8.8 | nfsA⁻ | 2250 |
| Dinitroaziridinylbenzamide | >200 | nfnB⁺ | 3.3 | nfnB⁺ | 3.3 | nfnB⁻ | >200 |
| (CB1954) | | nfsA⁺ | 6.3 | nfsA⁺ | 6.3 | nfsA⁻ | >200 |
| 3,5-Dinitrobenzamide | 250 | nfnB⁺ | 15.6 | nfnB⁺ | 15.6 | nfnB⁻ | >500 |
| | | nfsA⁺ | 7.8 | nfsA⁺ | 7.8 | nfsA⁻ | >500 |
| Fludarabine | >500 | deoD⁺ | 125 | deoD⁺ | 125 | deoD⁻ | >500 |
| 5-fluorocytosine | >2500 | codA⁺ | 625 | codA⁺ | 625 | codA⁻ | >2500 |
| Erythromycin | 200 | nfnB⁺ | 1.56 | nfnB⁺ | 200 | | |
| | | nfsA⁺ | 1.56 | nfsA⁺ | 200 | | |
| | | deoD⁺ | 1.56 | deoD⁺ | 200 | | |
| | | codA⁺ | 1.56 | codA⁺ | 200 | | |

These results are especially encouraging, given that all tested compounds are amphipathic compounds containing cationic groups, which are substrates of MDRs (Hsieh et al. (1998) Proc. Natl. Acad. Sci. USA 95:6602-6606; Lewis, K. (1999b) Curr. Biol. 9:R403-R407; Stermitz et al. (2000a) Proc. Natl. Acad. Sci. USA 97:1433-1437) (Lewis et al. (2002) Drug Efflux, in Bacterial Resistance to Antimicrobials: Mechanisms, Genetics, Medical Practice and Public Health. New York, Marcel Dekker, pp. 61-90). This is the first observation demonstrating an insensitivity of an amphipathic compound to MDR efflux in a Gram negative species, and suggests that the screen developed will lead to broad-spectrum compounds. It appears that the prodrug approach may be able to cut through the presently insurmountable obstacles of multidrug tolerance and MDR efflux.

### Example 8

### Screening for Sterilizing Prodrug Compounds

The bactericidal ability of the hits is then examined. This test probes the potential power of the screen to discover compounds capable of sterilizing an infection. While this is not a necessary property for an antibiotic, an ability to sterilize an infection is clearly advantageous, and especially important in treating persistent biofilm infections and in biodefense applications.

The currently accepted measure of a killing ability of an antibiotic is MBC, the concentration of a drug capable of decreasing the level of cells in a logarithmically-growing population by ≧3 orders of magnitude. The experiment is performed as a usual MIC broth microdilution assay in microtiter plates, and the first, and two subsequent wells that show no visible growth are plated for colony counts to determine the MBC. The definition of MBC, while arbitrary, is useful in gauging the bactericidal ability of an antimicrobial compound. However, this test completely misses the persister cells present in all populations, and is inapplicable to stationary and biofilm cultures Lewis, K. (2001a) Chemother. 45:999-1007; Coates et al. (2002) Nat. Rev. Drug Discov. 1:895-910). Most bactericidal antibiotics currently in use only act against rapidly growing cells. The FDA does not require testing developmental agents against stationary cultures, and the Industry does not perform such tests for pragmatic reasons—most compounds would likely fail. One of the results of this practice is the lack of compounds that are effective against biofilms, or other persistent infections.

For determining killing activity of hit compounds, the MBC is first measured by the standard broth microdilution method. Compounds that show considerable activity will be examined in detail, with the aim of evaluating their ability to kill non-growing populations and persister cells. For this, dose-dependent killing experiments will be performed with both log and stationary cultures of the wild type, and the strain overexpressing the cognate activating enzyme. Killing of non-growing cells is monitored by measuring the decline of viable cells of a stationary state population. The characteristic biphasic death observed with conventional antibiotics results from surviving persisters (Moyed et al. (1983) J. Bacteriol. 155:768-775; Spoering et al. (2001) J. Bacteriol. 183:6746-6751), and complete eradication of the culture indicates a sterilizing agent. Compounds that are able to kill non-growing cells and persisters in a wild type stationary population are of particular interest. Sterilization of an overexpression mutant, but not the wild type, will also indicate a promising prodrug whose properties (primarily affinity to the activating enzyme) could be further improved through medicinal chemistry.

### Example 9

### Killing Ability of Metronidazole

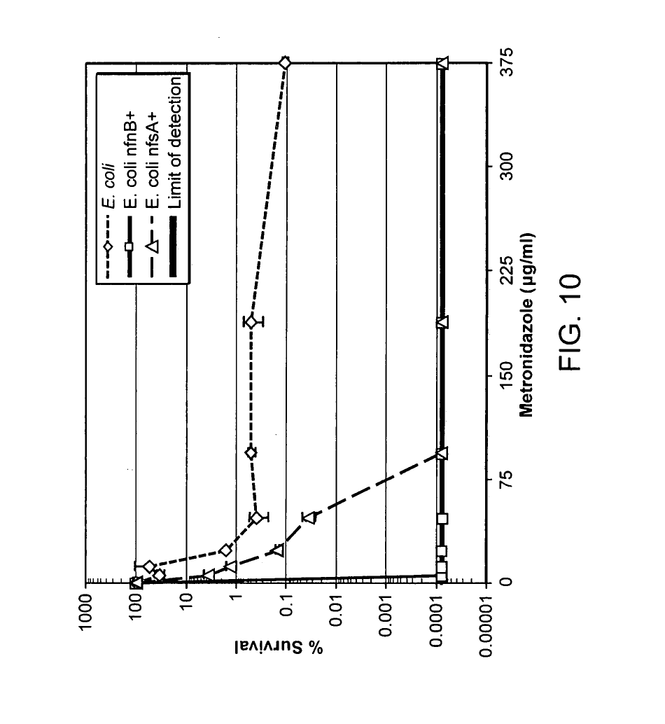

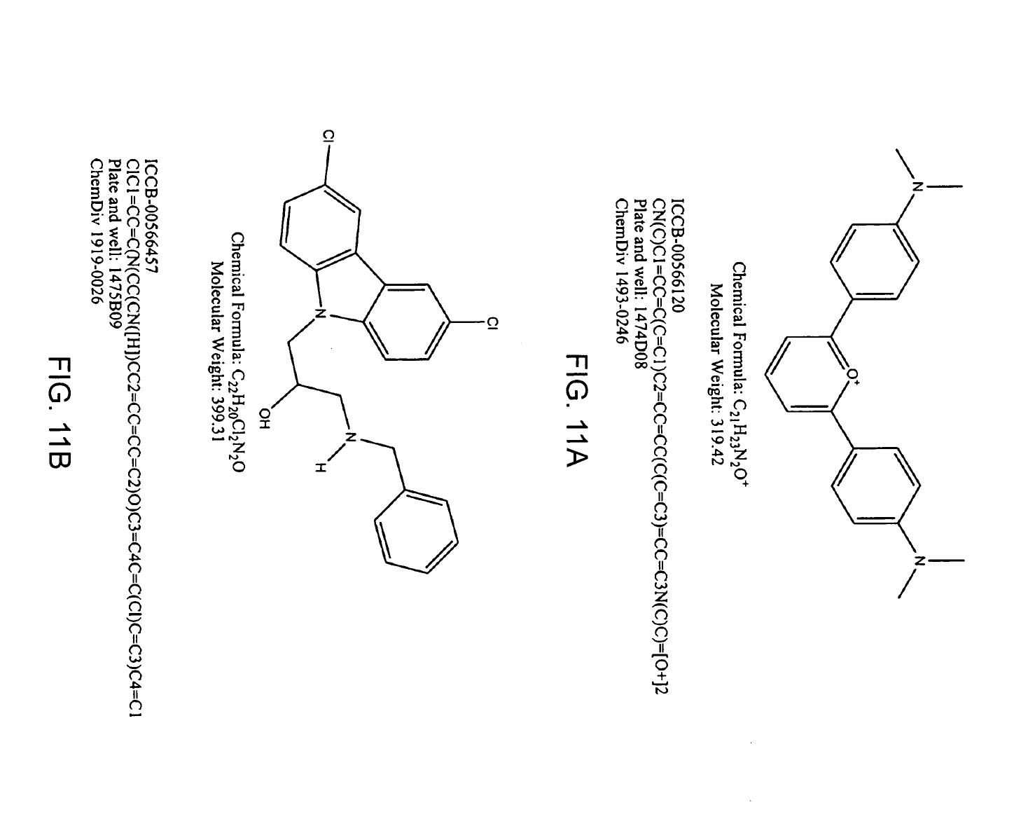

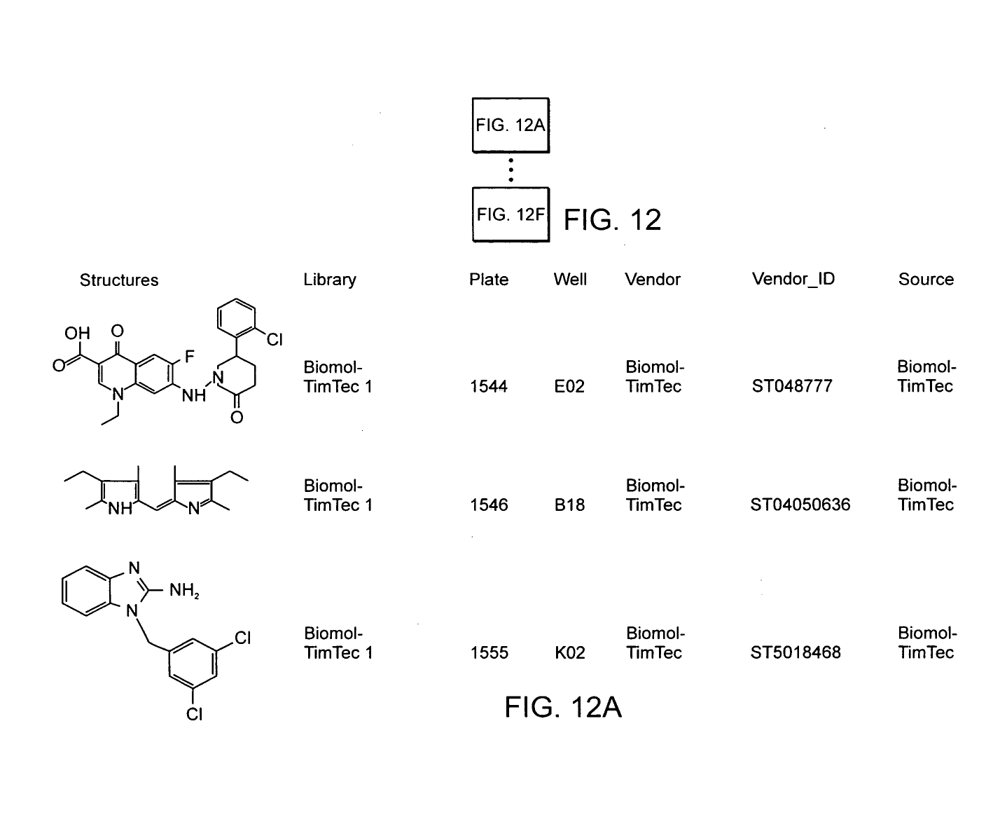

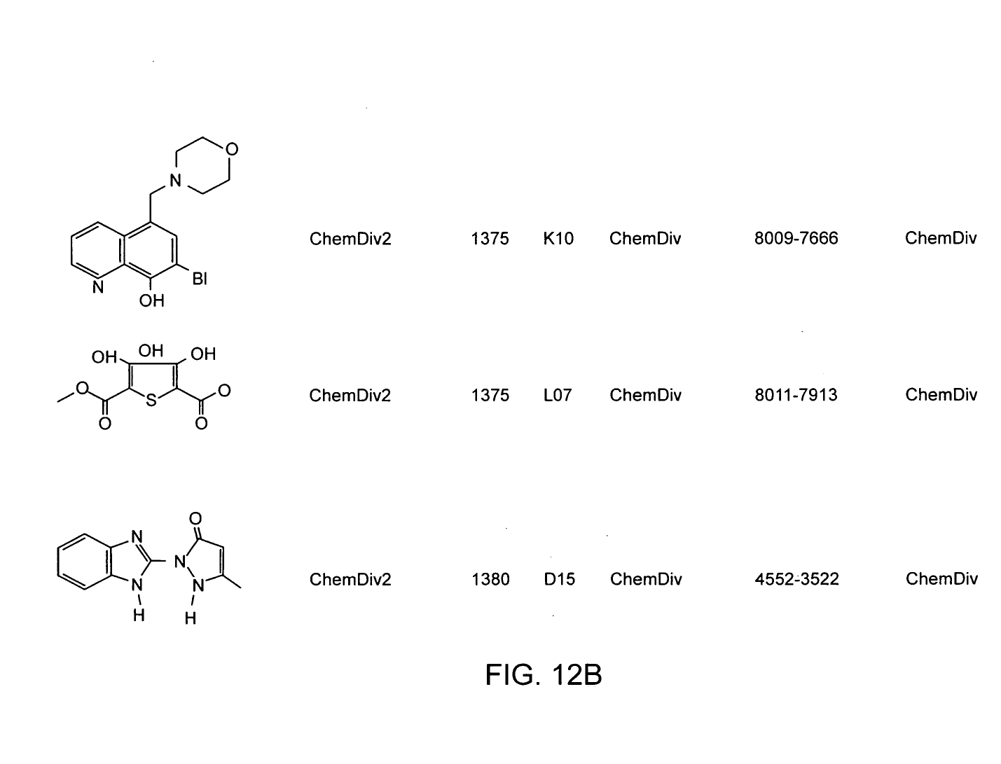

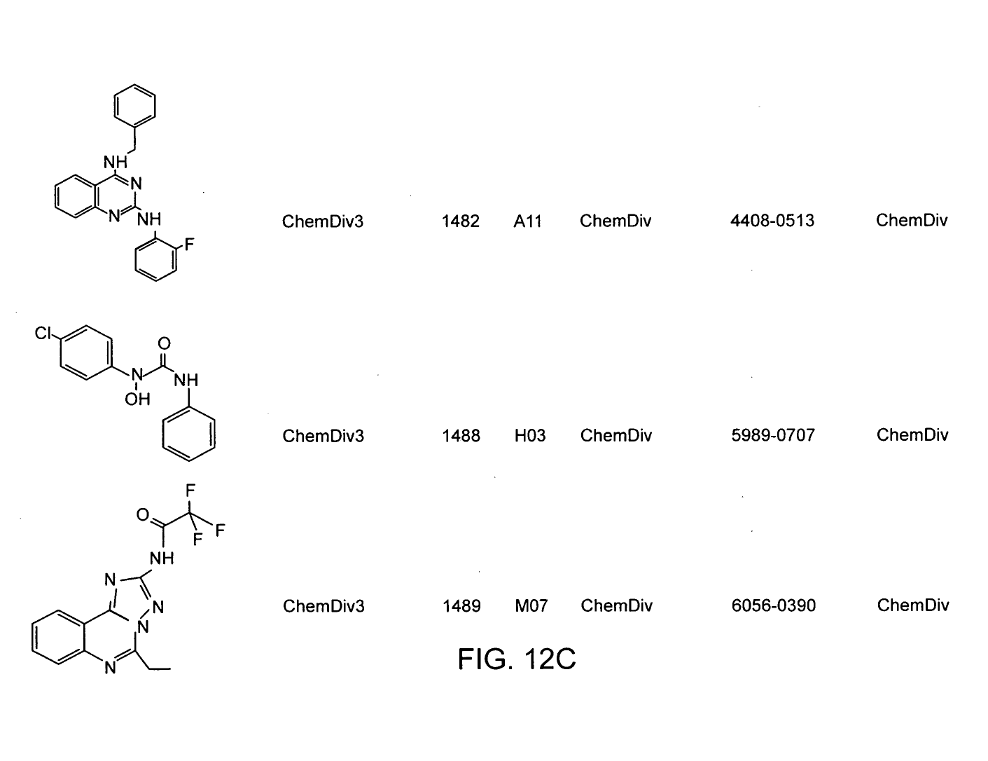

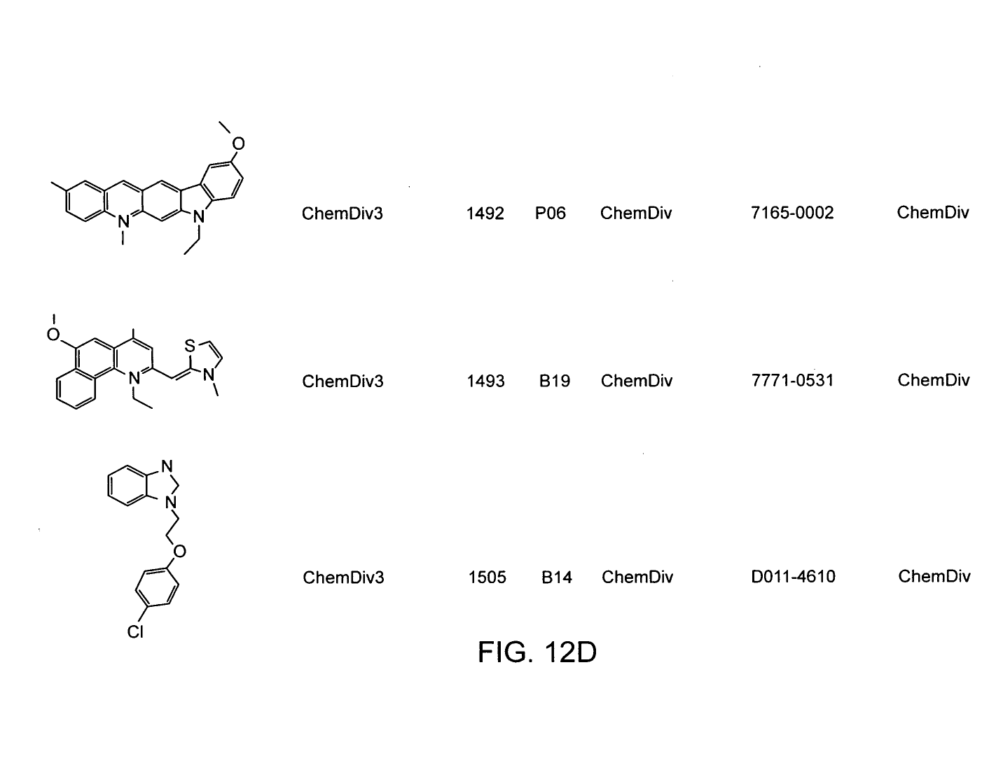

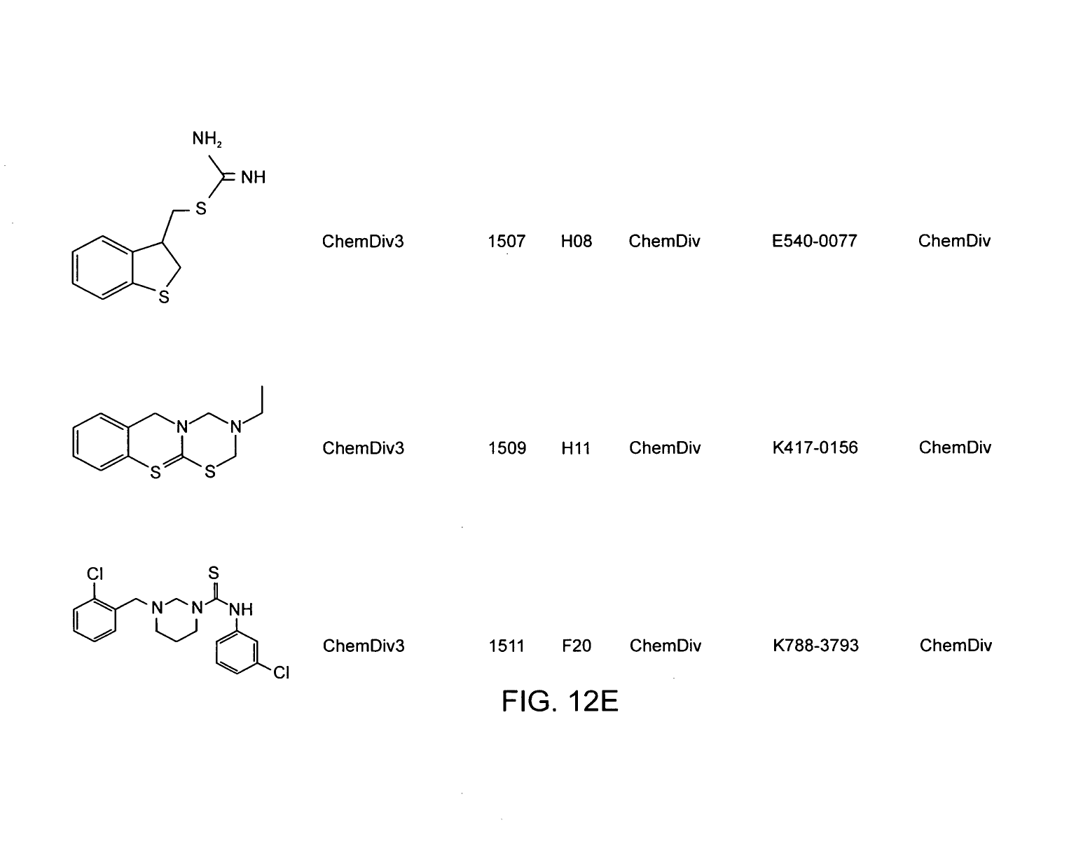

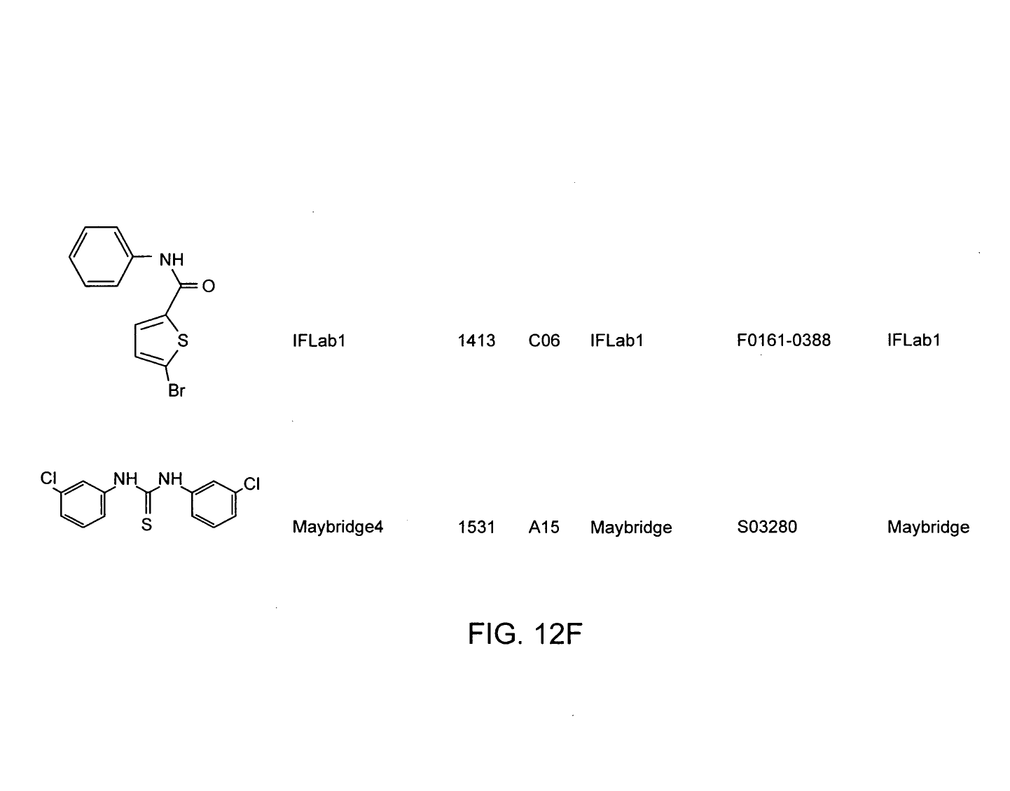

The killing ability of metronidazole was examined. The wild type bacterial strain was grown to stationary state under anaerobic conditions, and dose-dependent killing after incubation with metronidazole for 6 hours was detected by plating and colony count (FIG. 10). The wild type strain showed a typical biphasic killing, with ˜1% of tolerant persister cells. Interestingly, no surviving persisters were detected in a strain overexpressing the activating enzyme, NfnB or NfsA. A complete sterilization of the population was observed with metronidazole tested against the nfnB⁺ strain (the line corresponding to 6 logs of killing is the limit of detection, <10 cells/ml).

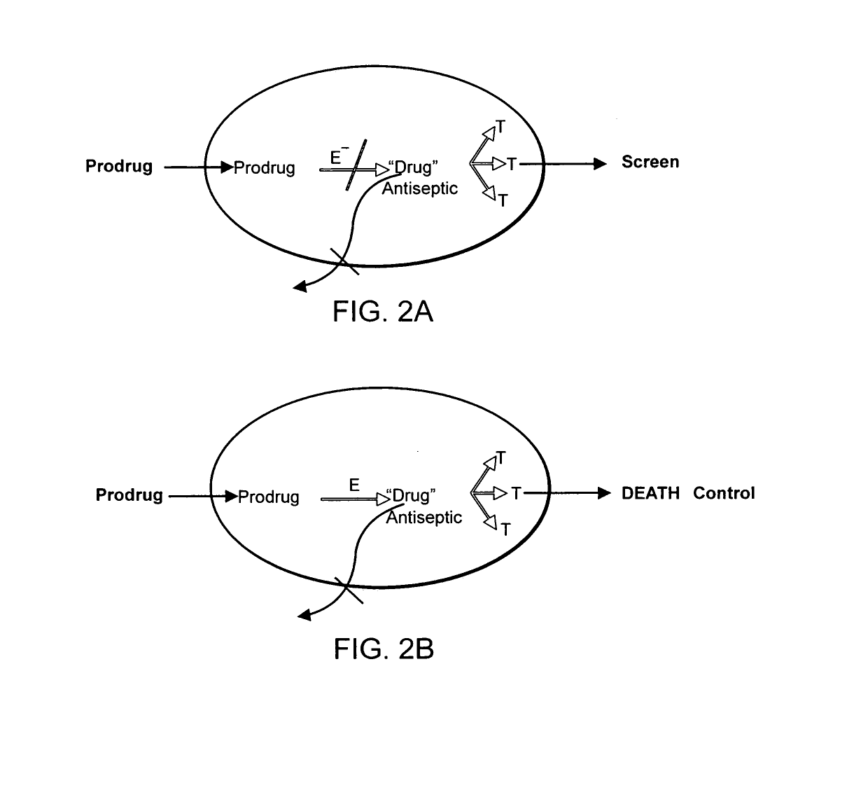

This to our knowledge is the first observation of a sterilizing activity of an antibiotic against a stationary state bacterial population. Previously, it was only possible to kill persisters and sterilize an infection with peracetic acid (FIG. 2); (Spoering et al. (2001) J. Bacteriol. 183:6746-6751). This experiment suggests that finding a prodrug with a better fit to its activating enzyme than metronidazole will produce an outstanding therapeutic.

This striking result demonstrates the potential power of prodrugs to sterilize infections in immunocompromised patients, and to solve the intractable problem of multidrug tolerant infections such as biofilms. Candidate compounds that come out of the proposed screen will be developed into drugs capable of sterilizing a broad range of pathogen infections.

### Example 10

### Persister Cells

In this example, multidrug tolerance of persister cells, which exemplifies the limitations of existing antibiotics, is characterized.

Persister cells in planktonic and biofilm populations are characterized for their antibiotic sensitivity in this example. Several bactericidal antimicrobials were chosen to test the resistance of P. aeruginosa to killing—ofloxacin, a fluoroquinolone; tobramycin, an aminoglycoside; carbenicillin, a β-lactam; and peracetic acid, a disinfectant oxidant (Spoering et al. (2001) J. Bacteriol. 183:6746-6751). Biofilms were grown essentially by the method of Ceri (Ceri et al. (1999) J. Clin. Microbiol. 37:1771-1776) and as described in Brooun et al. (2000) Antimicrob. Agents Chemother. 44:640-646. The device used for biofilm formation is a platform carrying 96 polystyrene pegs that fit in a microtiter plate. Preformed biofilms were incubated in the presence of an antimicrobial agent, and survival was measured after disrupting the biofilms by colony count.

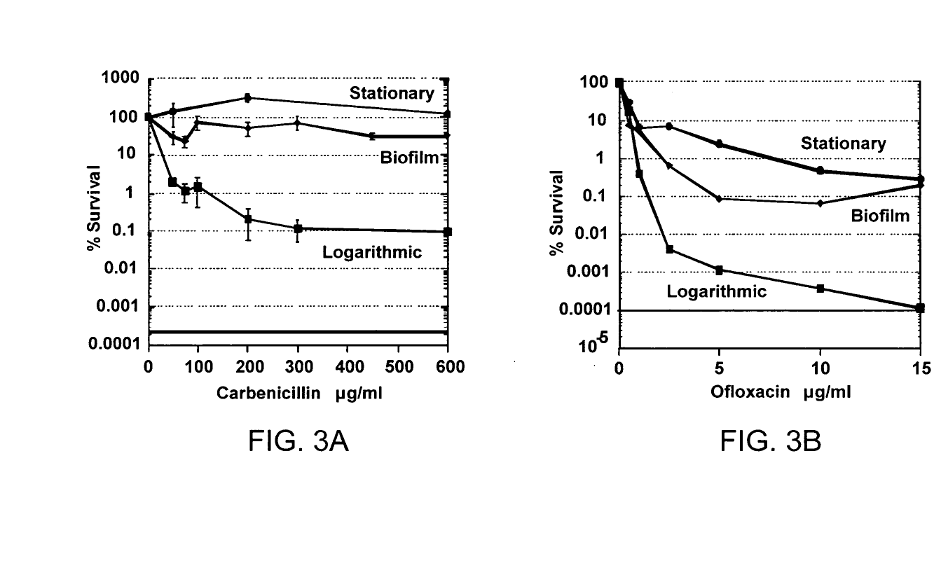

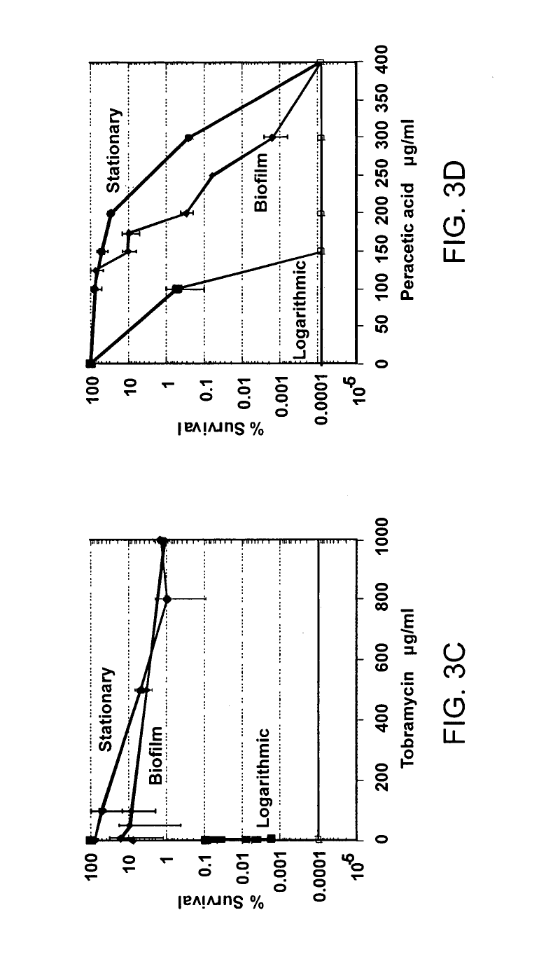

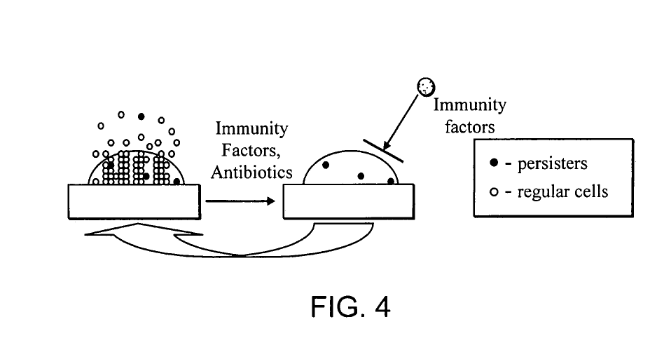

Carbenicillin is a bactericidal antibiotic that, similarly to most β-lactams, only kills rapidly growing cells (Tuomanen et al. (1986) Scand. J. Gastroenterol. Suppl:10-14). Logarithmic state, stationary and biofilm cultures were challenged with carbenicillin. As expected, carbenicillin produced little killing in stationary cells, while the majority of logarithmic cells were killed at 1.67×MIC (FIG. 3a).

The amount of killing of logarithmic cells approached a plateau at concentrations above 1.67×MIC, indicating the presence of a persister subpopulation. These 0.1% cells in the rapidly growing logarithmic culture were invulnerable to killing by carbenicillin at 600 μg/ml. Biofilm cells were resistant to killing by carbenicillin. This indicates that biofilms are made of slow growing cells.

Unlike carbenicillin, ofloxacin can kill non-growing cells (Brooun et al. (2000) Antimicrob. Agents Chemother. 44:640-646). Logarithmic, stationary and biofilm cultures were challenged with ofloxacin over a wide range of concentrations, from 1×MIC (0.5 μg/ml) to 30×MIC (15 μg/ml). After a 6 hours incubation time with the antibiotic, viability was determined by colony count. The majority of cells in the three populations examined were killed at low concentrations of ofloxacin (FIG. 3b). The killing in all three cultures was distinctly biphasic, indicating the presence of persister cells. The levels of persisters were dramatically higher in the dense stationary planktonic and biofilm cultures as compared to logarithmic cells. The plateau at increasing concentrations of antibiotic shows that persisters are essentially invulnerable to killing by a fluoroquinolone. At 5 μg/ml ofloxacin, which is the clinically achievable concentration (Schulz, M et al. (1997) Pharmazie 52:895-911), the percentage of live cells was 0.001% in the logarithmic population, 0.1% in the biofilm and 2.5% in the stationary culture (FIG. 3b).

Logarithmic phase, stationary phase and biofilm cultures were challenged with tobramycin over a wide range of concentrations, from 1×MIC (1 μg/ml) to 1500×MIC (1500 μg/ml). Tobramycin was exceptionally effective in killing logarithmic cells and no logarithmic persisters were detected (FIG. 3c). Tobramycin at 50 μg/ml (the maximal clinically achievable concentration is 10 μg/ml), (Schulz, M et al. (1997) Pharmazie 52:895-911) eliminated 90% of the biofilm cells, but the remaining population declined very gradually with increasing amounts of the antibiotic, and at high concentrations surviving persisters became apparent. Tobramycin was ineffective against stationary planktonic cells, apparently due to the dependence of killing of growth.

Peracetic acid, an oxidizing disinfectant, was used in order to test the ability of a sterilizing agent to act against persisters. Biphasic killing was not observed for this antimicrobial agent (FIG. 3d), and all cultures were sterilized. It appears that antibiotics acting against specific targets are inactive against persisters, and their elimination requires a general disinfectant/antiseptic compound. Similar results showing biphasic killing, and high level of persisters in stationary and biofilm populations, were obtained with S. aureus and E. coli as well (Keren et al. (2004) FEMS Microbiol. Lett. 230:13-18).

The data described above provide an insight into the recalcitrant nature of biofilm infections. Antibiotics like ofloxacin eliminate most of the cells of an infecting biofilm but leave persisters intact (FIG. 3). The immune system is likely to eliminate the remaining planktonic persisters (similarly to eliminating live planktonic cells after treatment with a static antibiotic). However, biofilm cells are physically protected from the components of the immune system by the exopolymer matrix (Hoyle et al. (1990) J. Antimicrob. Chemother. 26:1-5; von Eiff et al. (1999) Eur. J. Clin. Microbiol. Infect. Dis. 18:843-846). Leukocytes that partially penetrate the biofilm do not do much damage to bacterial cells (Leid et al. (2002) Infect. Immun. 70:6339-6345). Eradication of planktonic cells eliminates the symptoms of disease, and antibiotic treatment is discontinued. Once the antibiotic level drops, persisters reform the biofilm, which sheds off new planktonic cells. This model explains the relapsing nature of biofilm infections, and the need for a lengthy antibiotic therapy (Lewis, K. (2000) Microbiol. Mol. Biol. Rev. 64:503-514, Lewis, K. (2001a) Antimicrob. Agents Chemother. 45:999-1007). Other persistent infections, for example, non-biofilm infectious diseases in immunocompromised individuals, are likely to follow a similar pattern of population regrowth stemming from surviving persister cells.

### Example 11

### Multidrug Tolerance Genes in E. coli

Persisters are apparently dormant cells (Balaban et al. (2004) Science 305:1622-1625; Keren et al. (2004) FEMS Microbiol. Lett. 230:13-18), and this was tested directly by examining their capability for protein synthesis.

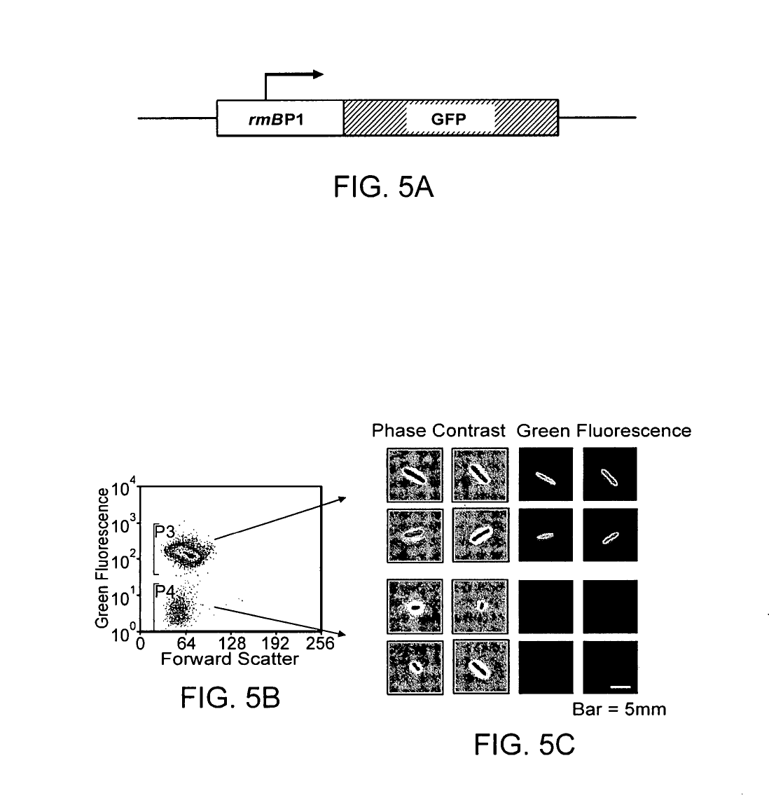

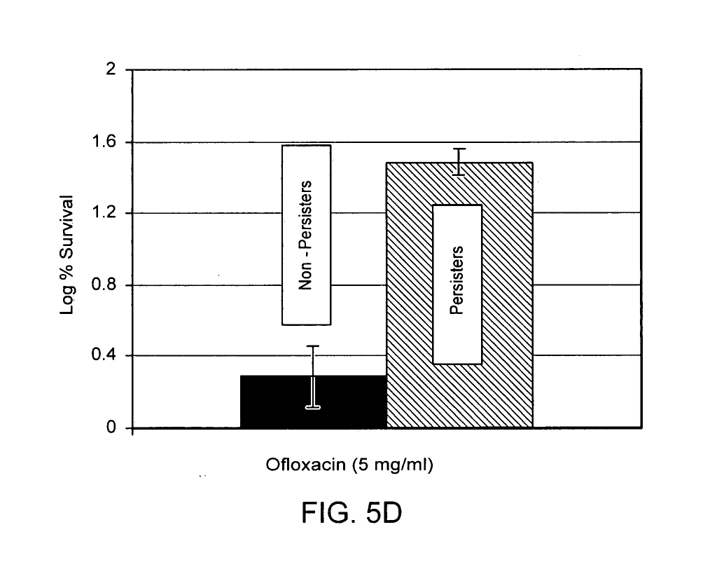

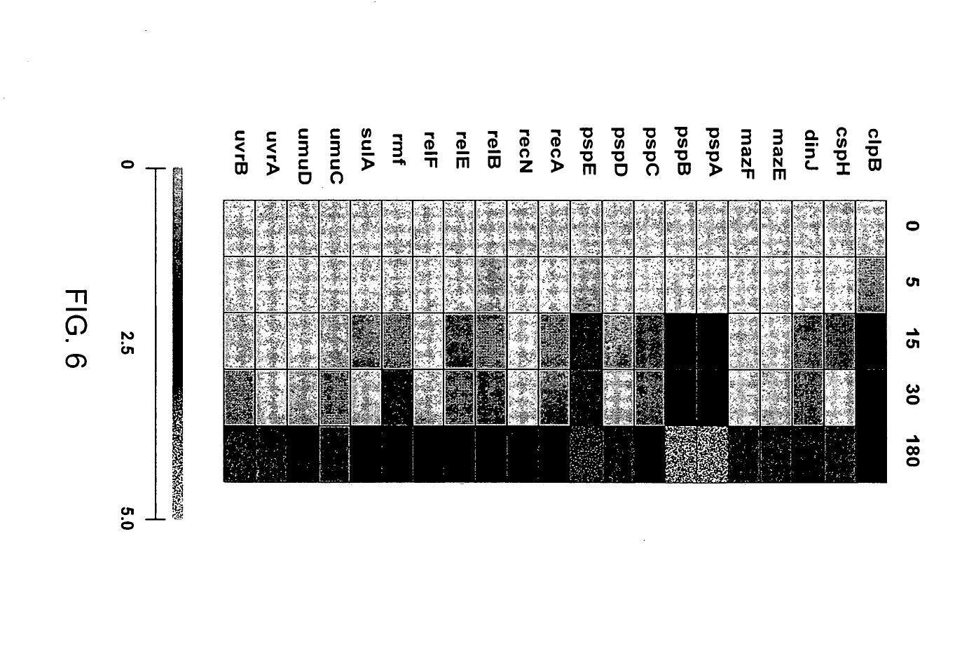

In E. coli ASV, a degradable GFP is inserted into the chromosome in the λ attachment site and expressed from the ribosomal rrnBP 1 promoter, the activity of which is proportional to the rate of cell growth (FIG. 5A). The half-life of degradable GFP is greater than 1 hour, and it should be effectively cleared from dormant cells. This would then enable sorting of dim persister cells.

A logarithmically-growing population of E. coli ASV was sorted with a MoFlo cell-sorter using forward light scatter, which allows detection of particles based on size. This enabled detection of cells irrespective of their level of fluorescence. Fluorescence of GFP in individual cells was recorded simultaneously using laser excitation and light detection. FACS analysis showed that the population consisted of two strikingly different types of cells—a bright majority, and a small subpopulation of cells with no detectable fluorescence (FIG. 5B). The two populations were sorted based on fluorescent intensity and collected in phosphate buffer. Epifluorescent microscopy confirmed that the sorted bright cells were indeed bright green, while the dim ones had no detectable fluorescence (FIG. 5C). The dim cells were also smaller than the fluorescent cells, and in this regard resembled stationary state cells. Sorted dim cells were exposed to a high level of ofloxacin that rapidly kills both growing and non-growing normal cells, but has no effect on persisters (Spoering et al. (2001) J. Bacteriol. 183:6746-6751; Keren et al. (2004) FEMS Microbiol. Lett. 230:13-18). The majority of this subpopulation survived, as compared to a drastic drop in viability of the sorted bright cells (FIG. 5D). This experiment shows that the sorted dim cells are dormant persisters.

To identify candidate persister genes, an expression profile from persister cells was identified. This was done both from sorted cells (in analysis) and from persisters collected after lysis of a growing population with ampicillin (Keren et al. (2004) J. Bacteriol. 186:8172-8180). Genes expressed in persisters that could create a dormant state were sought. The profile indicated several candidates—RMF inhibits translation by forming ribosome dimers (Wada, A. (1998) Genes Cells 3:203-208); UmuDC has been reported to inhibit replication (Opperman et al. (1999) Proc. Natl. Acad. Sci. USA 96:9218-9223); and SulA is an inhibitor of septation (Walker, G. C. (1996) Cell Mol. Biol. Neidhardt, F. C. (ed). Washington, DC:ASM Press, pp. 1400-1416) (FIG. 6).

E. coli HM22 hipA7 cells were grown in LB medium to mid-exponential phase (about 5×10⁷ cells/ml) at 37° C. with aeration and treated with 50 μg/ml ampicillin. After the culture lysed, remaining persisters were sedimented and the isolated RNA was used for microarray analysis. The heatmap of expressed genes was generated with Spotfire Decisionsite 7.2.

Most striking however was the overexpression of well-characterized chromosomal toxin-antitoxin (TA) modules RelBE, MazEF, and DinJ/YafQ, a homolog of RelBE. Homologs of these genes are found on plasmids where they constitute a maintenance mechanism (Hayes, F. (2003) Science 301:1496-1499). The ability of “toxin” modules to reversibly block translation (Pedersen et al. (2002) Mol. Microbiol. 45:501-510; Christensen et al. (2003) J. Mol. Biol. 332:809-819) and replication (Gerdes et al. (2005) Nat. Rev. Microbiol. 3:371-382) makes them excellent candidates for multidrug tolerant (MDT) genes. By shutting down antibiotic targets, toxins could produce multidrug tolerant cells.

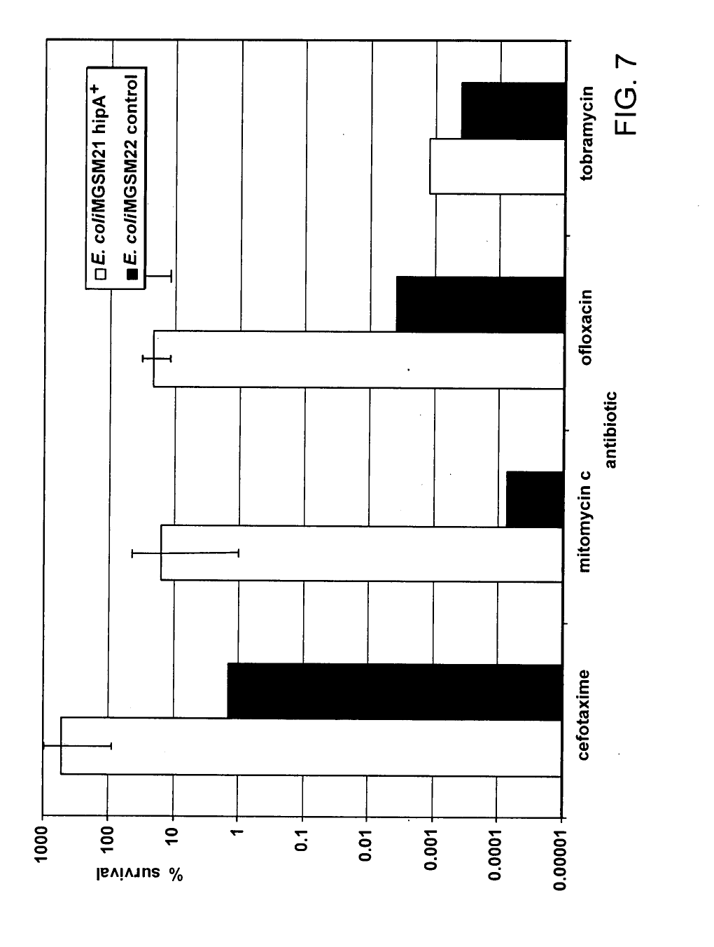

Overexpression of recombinant RelE increased the level of persisters surviving treatment with cefotaxime, ofloxacin and tobramycin 10-10,000 fold (not shown). Expression of another toxin, HipA, strongly protected cells from killing by antibiotics as well (FIG. 7).

### Example 12

### Multidrug Tolerance of E. coli Expressing HipA

Strains MGSM21(pBAD33::hipA) and MGSM22(pBAD33) were grown to OD600=0.3, at which point L-arabinose was added to induce expression of HipA from pBAD33. After 30 min a 1.0 ml aliquot of each strain was challenged with either cefotaxime (100 μg/ml), Mitomycin c (10 μg/ml), ofloxacin (5 μg/ml), or tobramycin (25 μg/ml) for 3 hours, at 37° C. with aeration. Cells were collected, washed once, diluted, and spot plated to determine CFU's.

After treatment with antibiotic, cells were plated on media without inducer and were allowed to recover. It is clear that persisters reverted to regular cells, overcoming the action of the toxins. How persisters resuscitate remains unknown.

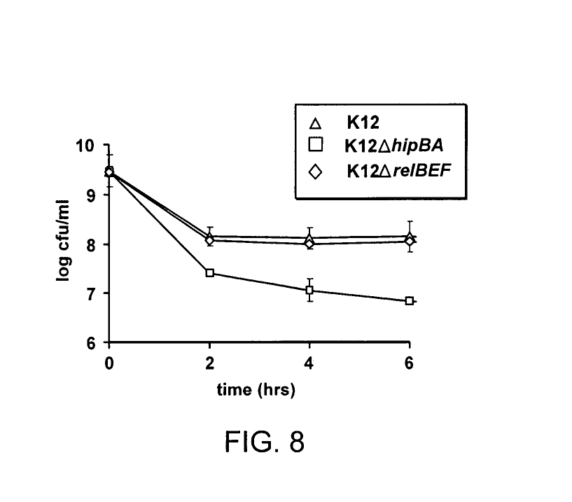

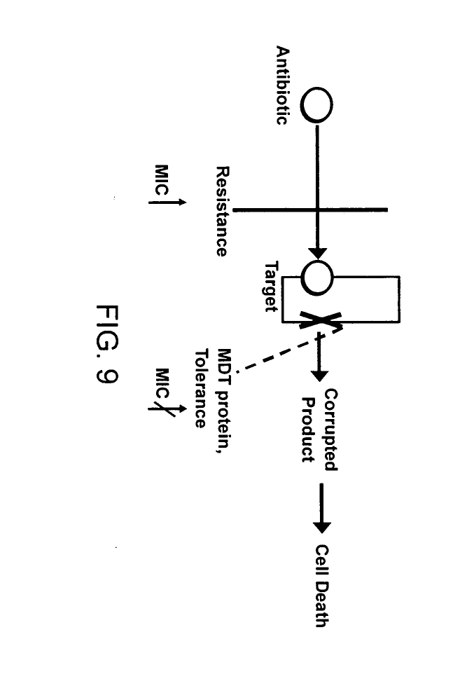

Strains deleted in relBE; mazEF; or hipBA were created Datsenko et al. (2000) Proc. Natl. Acad. Sci. USA 97:6640-6645) and tested for persister production in both growing and stationary cultures. Antibiotics exhibiting lethal action in stationary cultures are essentially limited to the fluoroquinolones and mitomycin C. Both antibiotics produced a sharp (10-100 fold) decrease in stationary cell persisters in the ΔhipBA strain (FIG. 8) and in a biofilm (not shown).

Deletion of hipBA had no effect on the MIC of antibiotics. When tested in a logarithmic culture, or in stationary state minimal medium, cells deleted in the hipBA locus did not show a lower level of persisters as compared to the isogenic parent strain (not shown). This agrees with a previous study, in which a hipBA null mutant was reported to have no phenotype when experiments were only performed with a growing culture (Black et al. (1991) J. Bacteriol. 173:5732-5739). Apparently, other MDT genes play a leading role in persister formation under those conditions. Deletion of either relBE (FIG. 8), mazEF, dinJ/yafQ, or rmf did not affect persister production (not shown). TA modules are highly redundant, and creating a multiply deleted strain will probably reveal the identity of additional MDT genes that play a role in logarithmic state cells.

Based on these findings, the following model of persister production and antibiotic tolerance is proposed (FIG. 9). The ratio of a toxin/antitoxin (such as HipA/HipB) in a population fluctuates, and rare cells express relatively high levels of a toxin. Bactericidal antibiotics bind to a target protein and corrupt its function, generating a lethal product (for example, aminoglycosides interrupt translation, resulting in misfolded peptides that damage the cell). A toxin binds to the target and inhibits the function, leading to tolerance. The antibiotic can bind to the blocked target, but can no longer corrupt its function. Inhibition of translation by a toxin further causes a relative increase in the stable toxin (due to antitoxin degradation) of this and other TA modules, which might have an autocatalytic effect on inhibition of translation, leading to a shutdown of other cellular functions, and to dormant, tolerant persister cells.

### Example 13

### E. coli .0157:H7 as an Exemplary Target Organism

E. coli O157:H7 is a gram negative human enterohemorragic (EHEC) pathogen that attaches to the surface of intestinal epithelium and results in a disease characterized by watery diarrhea, hemorrhagic colitis and hemolytic uremic syndrome (HUS) (Nataro et al. (1998) Emerg. Infect. Dis. 4:251-261; Paton et al. (1998) Clin. Microbiol. Rev. 1 1:450-479). E. coli O157:H7 is a Category B biodefense agent due to its exceptionally high virulence (100-200 cells) and water- and food-borne nature of the disease. The organism is easily transmitted from person to person and has been difficult to control, especially in child day-care centers. The two best known cases of E. coli O157:H7 outbreak occurred in USA between 1992 and 1993, affected more than 700 persons, 4 of which died (Bell et al. (1994) JAMA 272:1349-1353).

Pathogenicity involves a characteristic type III secretion system with attaching and effacing (A/E) lesions on intestinal epithelial cells, a microenvironment considered to be microaerophilic (Altier, C. (2005) J. Microbiol. 43:85-92), production of Shiga-like toxins, the third most deadly bacterial toxin in the world, and hemolysins. A 60 MDa plasmid present in E. coli O157:H7 is also responsible for production of an adhesin for Henle 407 intestinal cells, along with an hemolysin responsible for the hemorrhagic diarrhea (Karch et al. (1987) Infect. Immun. 55:455-461; Wadolkowski et al. (1990) Infect. Immun. 58:2438-2445). Symptoms of an E. coli infection usually appear 12 to 60 hours after consumption of the contaminated food(s) and normally last 5 to 10 days after onset. The most serious side effect of EHEC colonization is the Hemolytic and Uremic Syndrome (HUS) which is characterized by microangiopathic hemolytic anemia, capillary thrombosis and consequent ischemic necrosis. This complication seems to be due to EHEC Shiga-like toxins which most severely strike kidneys, leading to renal failure (Nataro et al. (1998) Emerg. Infect. Dis. 4:251-261) and ultimately death or chronic renal diseases which usually need artificial dialyses. In elderly the rate of mortality is estimated to be as high as 50%. As reported by the CDC, there are currently no licensed vaccines available for E. coli O157:H7, and the increasing antibiotic resistant strains of these food- and water-borne pathogens are emerging as a serious public health issue. The complete genome sequence of EHEC O157:H7 (Perna et al. (2001) Nature 409:529-533) revealed a large number of new genes likely to be involved in infection and disease.

### Example 14

### Animal Studies to Determine Pathogen Prodrug Activating Gene Function in a Host

The activating enzymes identified in the above screens are then examined for in vivo essentiality in a host. E. coli has a number of enzymes well conserved among bacteria, and it is likely that some of them are essential in the challenging environment of the host. An example of 30 well-conserved enzymes that do not have close human homologs, but are non-essential in vitro is given below (Table 7).

The in vivo essentiality of the activating enzymes that are identified is tested following the rate of clearance of E. coli O157:H7 in a mouse model of gastrointestinal infection. Disruptions of these genes in E. coli O157:H are made by the Wanner procedure (Keren et al. (2004) FEMS Microbiol. Lett. 230:13-18).

TABLE 7
Conserved Non-Essential E. coli Enzymes.

| E. coli | Mtub | Essential | Human | Annotation |
|---|---|---|---|---|
| Alr | Rv3423c | No | No | Alanine racemase |
| AroA | Rv3227 | No | No | 5-enolpyruvylshikimate-3-phosphate |
| | | | | synthase |
| AroB | Rv2538c | No | No | 3-dehydroquinate synthase |
| AroE | Rv2552c | No | No | Shikimate dehydrogenase |
| HisB | Rv1601 | No | No | Imidazoleglycerol-phosphate dehydratase |
| HisD | Rv1599 | No | No | Histidinol dehydrogenase |
| HisF | Rv1605 | No | No | Imidazoleglycerol-phosphate synthase |
| HisG | Rv2121c | No | No | ATP phosphoribosyltransferase |
| HisH | Rv1602 | No | No | Glutamine amidotransferase |
| MazG | Rv1021 | No | No | Predicted NTP pyrophosphatase |
| Mqo | Rv2852c | No | No | Malate: quinone oxidoreductase |
| PaaD | Rv1466 | No | No | Predicted component of oxygenase/ring |
| | | | | hydroxylase |
| Pta | Rv0408 | No | No | Phosphotransacetylase |
| TldD | Rv2315c | No | No | Microcin B17 maturation protease |
| WbbL | Rv3265c | No | No | Rhamnosyl transferase |
| YaeI | Rv3683 | No | No | Predicted phosphodiesterase |
| YcdH | Rv3567c | No | No | Flavin reductase |
| YedY | Rv0218 | No | No | Sulfite oxidase-relate molybdoenzyme |
| YfgB | Rv2880c | No | No | Pyruvate formate lyase activating enzyme |
| YgfJ | Rv0371c | No | No | MobA paralog, predicted molybdopterin- |
| | | | | guanine dinucleotide biosynthesis enzyme |
| YgiN | Rv2749 | No | No | Predicted monooxygenase |
| YhbJ | Rv1421 | No | No | Predicted P-loop-containing kinase |
| YhgH | Rv3242c | No | No | Predicted amidophosphoribosyltransferase |
| YidA | Rv3813c | No | No | Predicted phosphatase |
| YjbQ | Rv2556c | No | No | Predicted His,Asp-dependent enzyme |
| YjeQ | Rv3228 | No | No | Predicted GTPase |
| YjfR | Rv0906 | No | No | Predicted metallohydrolase (beta-lactamase |
| | | | | superfamily) |
| YjgR | Rv2510c | No | No | Predicted ATPase |
| YpfJ | Rv2575 | No | No | Predicted metalloprotease |
| YraL | Rv1003 | No | No | Predicted methyltransferase |
| ElaA | Rv2851c | Y/N | No | Predicted acyltransferase |
| YidD | Rv3922c | Y/N | No | Predicted Cys-dependent enzymes |
| HisI | Rv1606 | No | No | Phosphoribosyl-AMP cyclohydrolase |
| AbgA | Rv3305c | No | Y/N | Metal-dependent amidase/aminoacylase |
| AbgB | Rv3306c | No | Y/N | Metal-dependent amidase/aminoacylase |
| AroL | Rv2539c | No | Y/N | Shikimate kinase |
| CaiE | Rv3525c | No | Y/N | Carbonic anhydrase/acetyltransferase |
| MazG | Rv1021 | No | Y/N | NTP pyrophosphatase |
| MhpC | Rv3569c | No | Y/N | Predicted hydrolase/acyltransferase |
| YbaW | Rv2475c | No | Y/N | Predicted thioesterase |
| YcdJ | Rv0554 | No | Y/N | Predicted hydrolase/acyltransferase |

The animals are infected with the wild type, and the course of the infection is followed. As a control for essentiality, a strain with a chromosomal knockout of an essential dihydrodipicolinate reductase (dapB), expressing the enzyme from a regulated promoter on a recombinant vector is used. The plasmid is moved by transformation from E. coli K12 of the ASKA library into E. coli O157:H7. A knockout of the chromosomal copy is then made as described above. This strain is dependent on IPTG for growth, and is expected to be unviable in vivo. It is possible that leakage from the promoter is sufficient for this strain to grow in the absence of added inducer. This strain is not expected to cause disease, and should rapidly clear from the animals. Clearance is followed by plating samples on kanamycin medium (kan resistance is carried by the disruption cassette). In order to follow clearance of the test strains lacking activating enzymes, these are similarly plated on kanamycin medium. The rate of clearance of this strain serves as a benchmark to evaluate possible in vivo essentiality of the test strains. Those that fail to cause disease and exhibit clearance comparable to the dapB control will signify in vivo essentiality of the cognate gene. The hits that are converted by these in vivo essential enzymes are then entered into the drug development process. The strains overexpressing these in vivo essential enzymes are used together with strains overexpressing in vitro essential enzymes in a different modality of the screen.

Animals

6 to 8 week old female ICR (CD-1) mice are used for in vivo studies.

Bacterial Strains

Enterohemorragic E. coli O157:H7 strain EDL 933 (ATCC 700927) which produces both Shiga-like cytotoxins (SLT-I and SLT-II), are used in the study and serve as a positive control. In order to test in vivo essentiality, a set of 30 strains deleted in conserved non-essential enzymes is prepared (Table 7) as described above. A strain carrying a disruption in an essential gene dapB and expressing it from a plasmid in response to IPTG serves as a negative control. Knockout strains (CAT^{R}) is grown in LB broth at 37 C for 16 to 18 hr, diluted 1/1000 in fresh LB broth, cultured to mid-log phase, harvested by centrifugation, washed twice in phosphate-buffered saline (PBS, pH 7.4) and resuspended in PBS. Chloramphenicol will be added to media at a final concentration of 25 μg/ml.

Gene Disruption

Chromosomal genes are disrupted using linear DNA fragments with short (˜50 bp) terminal homologies to the targeted gene(s) and phage λ Red recombinase. A gene cassette encoding kanamycin resistance is amplified by PCR using primers with 5′-extensions that are homologous to regions adjacent to the targeted gene, and the PCR fragment will be electroporated into cells expressing Red recombinase encoded by a helper plasmid. Kanamycin resistant colonies with the resistance cassette integrated into chromosome are isolated and verified by PCR. The temperature sensitive helper plasmid is then cured.

Procedures

Animals are allotted one per cage and allowed to acclimate (free access to water and food) for at least three days upon arrival at the experimental facility. Animals are then starved for water and food for 18 hr, infected the next morning by intragastric gavage with 10⁸ cells of the desired E. coli O157:H7 strain and then allowed access to food and water ad libitum. Each strain will be tested in triplicate. The control strain expressing an essential IPTG-inducible DapB is expected to rapidly disappear from the animals. As a negative control, a group of animals receive only sterile PBS, while the positive control animals receive 10⁸ cells of the wild type E. coli O157:H7 EDL933 strain.

For the following 15 days animals are controlled daily for feed and water intake, weight, and general health status. Feces are collected for E. coli O157:H7 counts, dry-matter measurements, and fecal occult blood detection.

At days 5, 10 and 15 three animals per each group are euthanized via CO₂ inhalation and samples of: mucosa from proximal, middle and distal small intestine, cecum, and proximal, and distal colon, feces, urine and blood will be collected for E. coli O157:H7 counts. At the same time each mouse undergo a full-necroscopy examination and specimens of: proximal, middle and distal small intestine, cecum, colon, liver, spleen and kidneys are collected, fixed in 10% buffered neutral formalin and processed for further histological examinations.

Expected Results

The in vivo experiments point out possible in vivo essentiality of “in vitro non essential genes,” by tracking clearance (survival) of knockout strains of E. coli O157:H7 EDL933. At the same time the sampling and analysis procedures allows a determination of the role of tested genes in: time-course of infection establishment, severity of disease, ability of E. coli O157:H7 EDL933 to colonize intestinal mucosa and degree of intestinal lesions, fecal shedding of the pathogen, as well as systemic lesion in more sensitive organs (kidneys, liver and spleen) and possible septicemia occurrence. Overtime observations of animals' behavior relative to feed and water intake, general health status and detection of occult blood in feces reliably allow to recognize occurrence of a sub-clinical status of infection.

### Example 15

### Prodrug Antibiotic Screens Using Essential Genes

Strains Diminished in Expression of Essential Enzymes

In order to focus specifically on the essential activating enzymes, a set of strains with diminished expression is constructed. As mentioned above, decreased expression of an essential target leads to an increase in activity of a conventional antibiotic, and this property has been used by the Industry for whole-cell screening for compounds hitting this target (Schmid, M. B. (2001) New targets and strategies for identification of novel classes of antibiotics. In Antibiotic Development and Resistance. Hughes, D. and Andersson, D. I. (eds). New York:Taylor and Francis, pp. 197-208). Diminished expression will lead to decreased activity of a prodrug, and this will serve to specifically identify these compounds, similarly to screening a knockout library described in (a).

There are ˜300 essential genes in E. coli (Gerdes et al. (2003) J. Bacteriol. 185:5673-5684), 250 of which are suitable enzymes (M. Galperin, unpublished). An antisense RNA approach is used to construct a set of E. coli strains with diminished expression of 250 essential enzymes. A large-scale construction of antisense strains has been successfully employed before to identify essential enzymes in S. aureus (Ji et al. (2001) Science 293:2266-2269). A similar strategy is utilized, and follows the specific protocols for antisense suppression of target genes developed for E. coli (Chen et al. (2003) Antimicrob. Agents Chemother. 47:3485-3493; Stefan et al. (2003) FEBS Lett. 546:295-299; Wang et al. (2003) FEMS Microbiol. Lett. 220:171-176). DNA fragments coding for a given enzyme are amplified by PCR. Primers carrying restriction sites are used, enabling cloning of the amplified DNA into pCA24N vector in the antisense orientation relative to the pT5lac promoter. Resulting constructs enable controlled expression of antisense RNA through induction with IPTG (see Methods for details). Strain construction is streamlined and cloning procedures performed in parallel in microtiter plates.

Addition of IPTG at a saturating concentration (3 μM) leads to a high level of antisense RNA expression. All recombinant strains are therefore be cultured in plates with 3 μM IPTG, and lack of growth signifies successful construction. Next, the level of IPTG is determined for each strain that decreases growth rate. This is performed in 384 well microtiter plates under conditions similar to screening. Each strain is inoculated into a well with one of 5 IPTG concentrations and a control. This results in a total of 1250 wells, or 4 plates. The experiment establishes the level of expression that still gives good growth (˜20% inhibition), suitable for screening, but a decreased level of an activating enzyme sufficient to provide increased resistance to a prodrug. This criterion of expression was used successfully by Microcide/Essential Therapeutics who employed strains with diminished expression of a target to specifically screen for compounds hitting that target by detecting increased activity (Schmid, M. B. (2001) New targets and strategies for identification of novel classes of antibiotics. In Antibiotic Development and Resistance. Hughes, D. and Andersson, D. I. (eds). New York:Taylor and Francis, pp. 197-208). The difference is that Microcide was interested in specific antibiotics, while this screen only selects prodrug hits that have decreased activity in this screen as compared to the wild type.

One of the 5 tested concentrations of IPTG will result in a suitable level of essential enzyme expression for most if not all of the strains. Given that different IPTG levels are required for optimal expression of different strains, a single mix cannot be used for screening, as was done in a pilot with the deletion set. Accordingly, the 250 strains expressing antisense RNA are grouped into 5 distinct sets, and each is screened separately. This means 5 separate screens of a 150,000 compound library. A more efficient format for this screen is also available. Prodrugs are found among compounds that have direct antimicrobial activity. Therefore, the 150,000 compound NERCE library is screened against wild type E. coli W3110. Based on previous experience, the hit rate for antimicrobials in a compound library against E. coli is ≦5%. Taking the higher estimate of 5%, this will result in 7,500 compounds with direct activity. These are then picked by a “cherry-picking” robot and reformatted into a sublibrary. Screening this sublibrary against 5 pools of strains and a wild type control will then be equivalent to a screen of 45,000 compounds.

Subsequent steps of verifying and validating the hits are similar to those described above. Briefly, the hits that allow growth in a pool of strains with diminished enzyme expression signify possible prodrug compounds. A secondary screen involves testing the hit compound against individual strains of the mix which identifies the strain resistant to a particular compound. Next, validation is performed with a strain from the ASKA library overexpressing the enzyme. An increased susceptibility against a strain overproducing an enzyme as compared to a strain with diminished expression serves as an initial validation for a prodrug. The wild type is incorporated in this test as well. This experiment is performed with a range of hit concentrations and will produce an MIC for the strains, including the wild type. Hits with the lowest wild type MIC are then focused on. The hits are then examined for their ability to avoid MDR efflux by testing their activity in a tolC background; and for their ability to sterilize a stationary state population, as described above.

Creating Strains with Controlled Gene Expression using an Antisense Approach

DNA fragments coding for the chosen enzymes are amplified by PCR using corresponding clones from the ASKA overexpression library as template and a pair of primers complementary to the vector sequence flanking the cloned ORFs. Primers also carry restriction sites enabling cloning the amplified enzyme coding inserts into pCA24N vector (GeneBank accession number AB052891) in the antisense orientation relative to the pT5lac promoter in the vector. Resulting constructs enable controlled expression of antisense RNA through the induction with IPTG. Tight repression before induction is ensured by the expression of the lacl^{q} gene cis-encoded on the same vector (Chen et al. (2003) Antimicrob. Agents Chemother. 47:3485-3493; Stefan et al. (2003) FEBS Lett. 546:295-299; Wang et al. (2003) FEMS Microbiol. Lett. 220:171-176).

Equivalents

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A method of inhibiting the growth of, or killing, a pathogen, comprising contacting the pathogen with one or more prodrug compounds of Formulae I, II, and III, 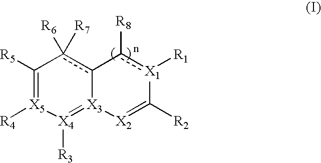
or a pharmaceutically acceptable salt, hydrate, or solvate thereof,
wherein
R¹ is null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups;
R₂ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl, 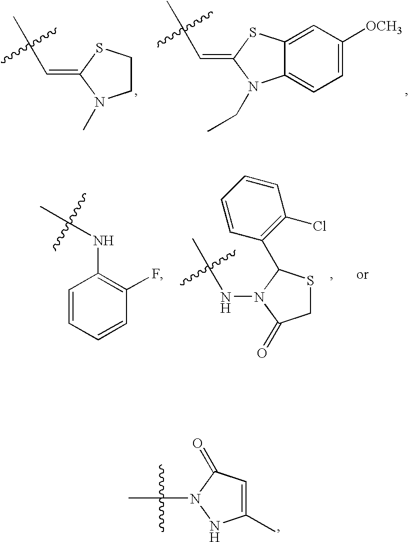
wherein all except —H can be substituted with 0-5 Rₐ groups; or R₁ and R₂ can be taken together to form a 6-membered aryl moiety that can be substituted with 0-4 Rₐ groups;
R₃ and R₄ are each independently null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups; or R₃ and R₄ can be taken together to form a 6-membered aryl moiety that can be substituted with 0-4 Rₐ groups;
R₅ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups; or R4 and R₅ can be taken together to form a 6-membered aryl moiety that can be substituted with 0-4 Rₐ groups;
R₆ and R₇ are each —H, or both R₆ and R₇ can be taken together to form a carbonyl;
R₈ is null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, heteroarylalkyl, 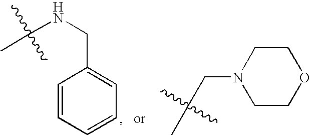
wherein all except H can be substituted with 0-5 Rₐ groups;
Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;
X₁, X₂, X₃, X₄, and X₅ are each independently —N—, —N⁺—, —C(R₁)—, or —C(H)—; [symbol]
denotes a single or double bond;
n is 0 or 1;
x is 0, 1, or 2; 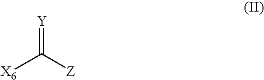
or a pharmaceutically acceptable salt, hydrate, or solvate thereof,
wherein
X₆ is NR₉R₁₀, or SR₁₁;
Y is NH, O, or S;
Z is NR₁₂R₁₃;
R₉, R₁₀, R₁₂, and R₁₃, are each independently —H, —OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, or C₃₋₆ cycloalkyl-C₁₋₃ alkyl, wherein all except H can be substituted with 0-5 Rₐ groups; or R₁₂ and R₁₃ can be taken together with the nitrogen to which they are attached to form a nitrogen containing 5- or 6-membered monocyclic heterocycle that can be substituted with 0-5 Rₐ groups;
R₁₁ is —H, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, or 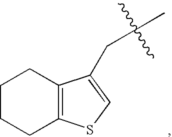
wherein all except H can be substituted with 0-5 Rₐ groups; or R₁₁ and R₁₂ can be taken together to form a 5- or 6-membered monocyclic heterocycle that can be substituted with 0-5 Rₐ groups;
Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;
x is 0, 1, or 2; 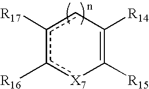
or a pharmaceutically acceptable salt, hydrate, or solvate thereof,
wherein
R₁₄, R₁₅, R₁₆ and R₁₇ are each independently —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl, or 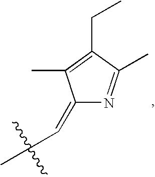
wherein all except H can be substituted with 0-5 Rₐ groups;
Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;
X₇ is NH, S, O, or O⁺; [symbol]
denotes a single or double bond whereby no more than two of [symbol] can be a double bond;
n is 0 or 1; and
x is 0, 1, or 2;
wherein contacting the pathogen with one or more prodrug compounds of Formulae I, II, and III inhibits or kills the pathogen.

2. The method of claim 1, wherein the prodrug compound is of Formula I.

3. The method of claim 1, wherein the prodrug compound is of Formula II.

4. The method of claim 1, wherein the prodrug compound is of Formula III.

5. The method of claim 1, wherein the pathogen is selected from the group consisting of a bacterium, a fungus, a protozoan, a helminth, and a combination thereof.

6. The method of claim 1, wherein the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

7. A method of inhibiting the growth of, or killing, a pathogen, comprising contacting the pathogen with one or more analogs of prodrug Compounds 1-19: 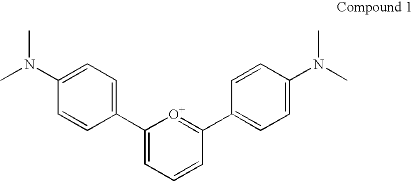
or a pharmaceutically acceptable salt, hydrate, or solvate thereof,
wherein any one or more of —H, and —N(CH₃)₂ can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 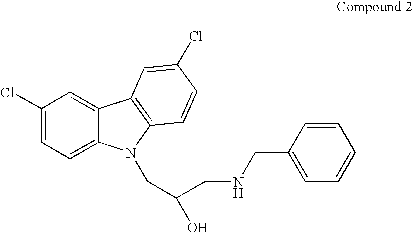
or a pharmaceutically acceptable salt, hydrate, or solvate thereof,
wherein any one or more of —H, —OH, —Cl, and —CH₂CH(OH)CH₂N(H)benzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 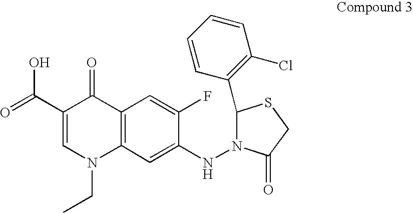
or a pharmaceutically acceptable salt, hydrate, or solvate thereof,
wherein any one or more of —H, —F, —CH2CH3, —C(O)OH, and chlorophenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 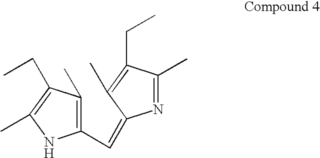
or a pharmaceutically acceptable salt, hydrate, or solvate thereof,
wherein any one or more of —H, —CH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the E configuration can be Z; 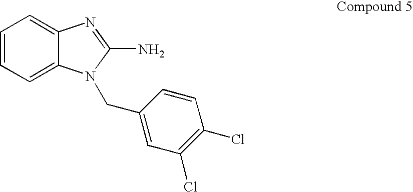
or a pharmaceutically acceptable salt, hydrate, or solvate thereof,
wherein any one or more of —H, —NH₂, and dichlorobenzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 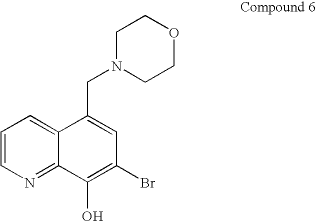
or a pharmaceutically acceptable salt, hydrate, or solvate thereof,
wherein any one or more of —H, —Br, —CH₂-morpholino, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 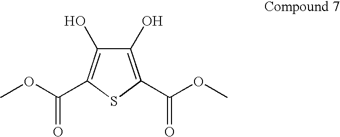
or a pharmaceutically acceptable salt, hydrate, or solvate thereof,
wherein any one or more of —H, —OH, —C(O)OCH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the thiophenyl —S— can be substituted with —O; 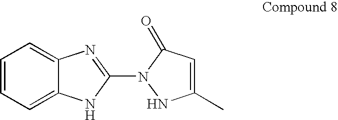
or a pharmaceutically acceptable salt, hydrate, or solvate thereof,
wherein any one or more of —H, and —CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 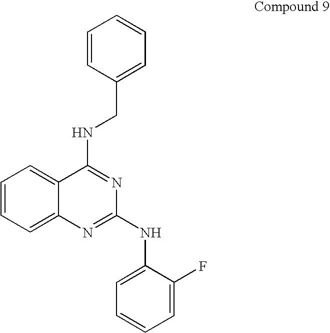
or a pharmaceutically acceptable salt, hydrate, or solvate thereof,
wherein any one or more of —H, —F, benzyl, and phenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 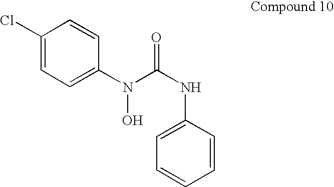
or a pharmaceutically acceptable salt, hydrate, or solvate thereof,
wherein any one or more of —H, —OH, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and —C(O) can be substituted with —C(S); 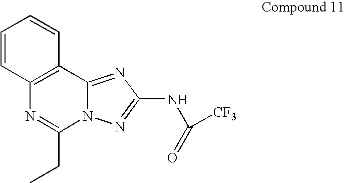
or a pharmaceutically acceptable salt, hydrate, or solvate thereof,
wherein any one or more of —H, —F, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 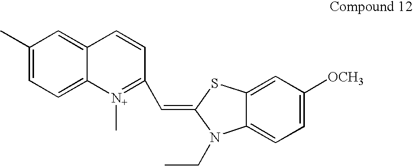
or a pharmaceutically acceptable salt, hydrate, or solvate thereof,
wherein any one or more of —H, —CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the Z configuration can be E; 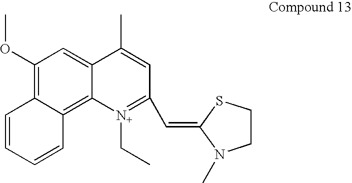
or a pharmaceutically acceptable salt, hydrate, or solvate thereof,
wherein any one or more of —H, CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the Z configuration can be E; 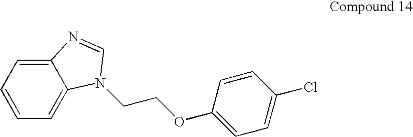
or a pharmaceutically acceptable salt, hydrate, or solvate thereof,
wherein any one or more of —H, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 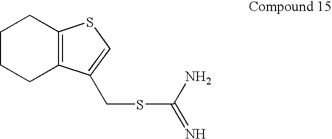
or a pharmaceutically acceptable salt, hydrate, or solvate thereof,
wherein any one or more of the following substituents, —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 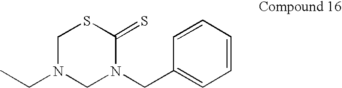
or a pharmaceutically acceptable salt, hydrate, or solvate thereof,
wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 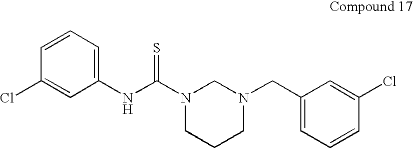
or a pharmaceutically acceptable salt, hydrate, or solvate thereof,
wherein any one or more of —H, —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; chlorophenyl can be substituted with heteroaryl, chlorobenzyl can be substituted with alkyl, alkylheteroaryl, and acyl; 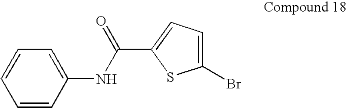
or a pharmaceutically acceptable salt, hydrate, or solvate thereof,
wherein any one or more of —H, and —Br, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; —C(O) can be substituted with —C(S), and S can be substituted with O; and 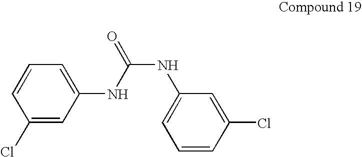
or a pharmaceutically acceptable salt, hydrate, or solvate thereof,
wherein any one or more of —H, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and —C(S) can be substituted with —C(O);
wherein contacting the pathogen with one or more analogs of prodrug Compounds 1-19 inhibits or kills the pathogen.

8. The method of claim 7, wherein the pathogen is selected from the group consisting of a bacterium, a fungus, a protozoan, a helminth, and a combination thereof.

9. The method of claim 7, wherein the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoebafragilis, Giardia lamblia, Cryptosporidium parvum, Naegleriafowleri, Acanthomeaba SPP, Isospora belli, Microsporidia, flatworms, and roundworms.

10. A method of inhibiting the growth of, or killing, a pathogen, comprising contacting the pathogen with one or more prodrug compounds of one or more of prodrug Compounds 1-19: 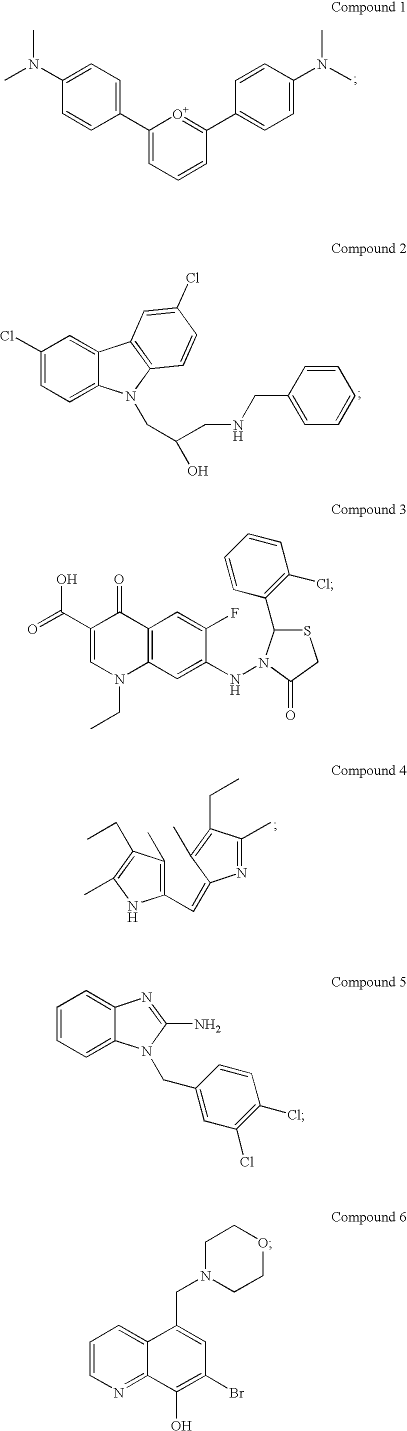 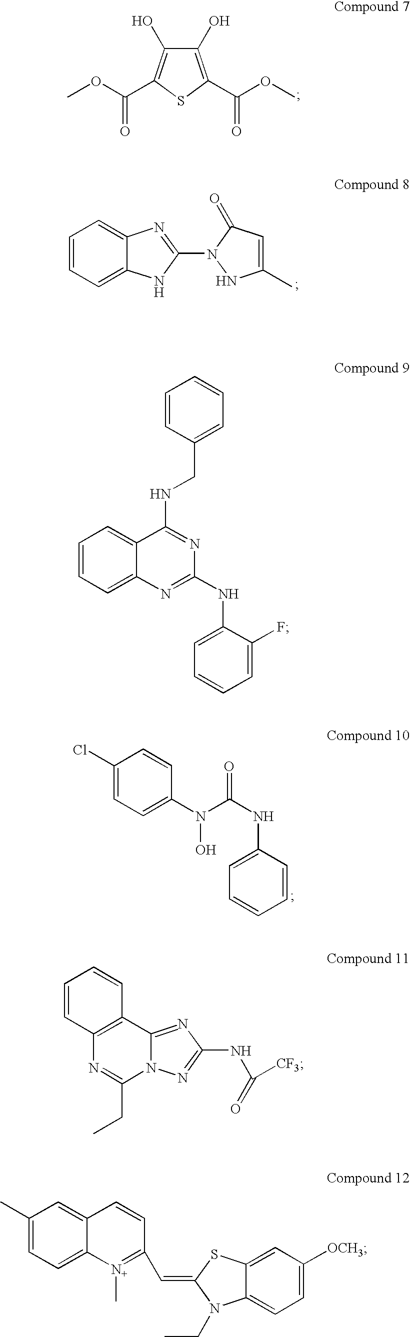 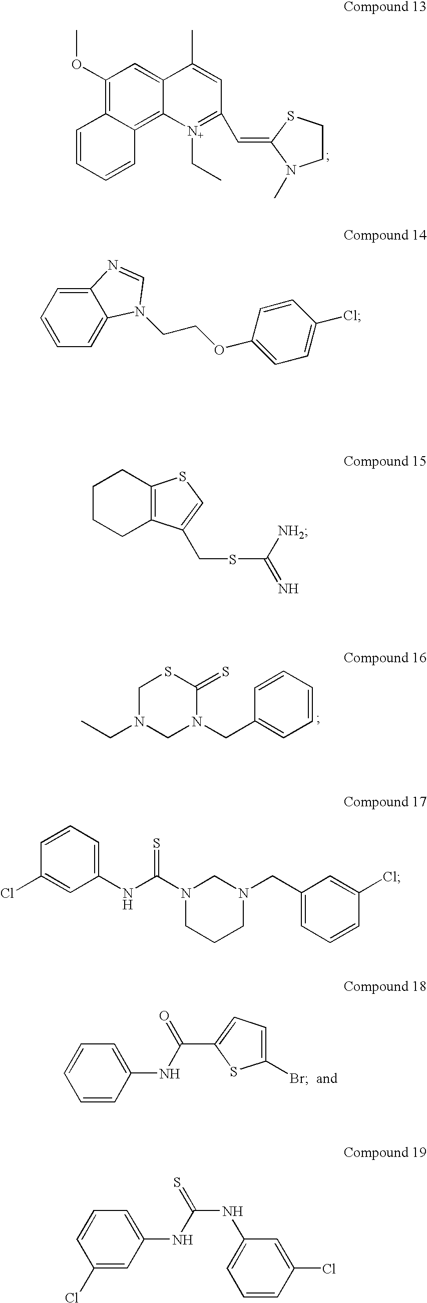
or a pharmaceutically acceptable salt, hydrate, or solvate of Compounds 1-19, wherein contacting the pathogen with one or more of prodrug Compounds 1-19 inhibits or kills the pathogen.

11. The method of claim 10, wherein the pathogen is selected from the group consisting of a bacterium, a fungus, a protozoan, a helminth, and a combination thereof.

12. The method of claim 10, wherein the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

13. A method of treating an infection by a pathogen in a patient in need thereof, the method comprising administering to the patient an effective amount of one or more prodrug compounds of Formulae I, II, and III, 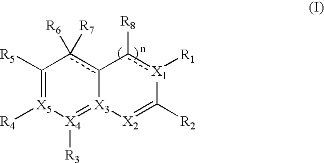
or a pharmaceutically acceptable salt, hydrate, or solvate thereof,
wherein
R¹ is null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups;
R₂ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —NHC(O)—C₁-C₆ alkyl, aryl, arylalkyl, heteroaryl, heteroarylalkyl, 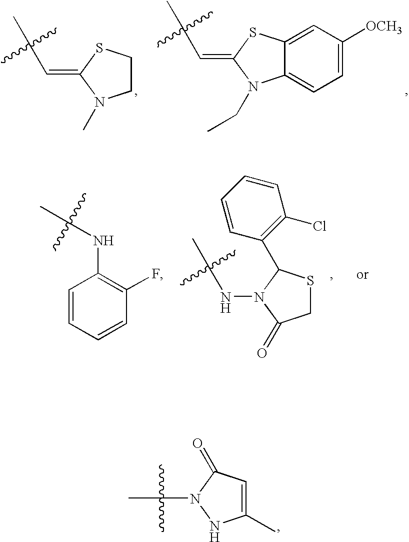
wherein all except —H can be substituted with 0-5 Rₐ groups; or R₁ and R₂ can be taken together to form a 6-membered aryl moiety that can be substituted with 0-4 Rₐ groups;
R₃ and R4 are each independently null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups; or R₃ and R₄ can be taken together to form a 6-membered aryl moiety that can be substituted with 0-4 Rₐ groups;
R₅ is —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, or heteroarylalkyl, wherein all except H can be substituted with 0-5 Rₐ groups; or R₄ and R₅ can be taken together to form a 6-membered aryl moiety that can be substituted with 0-4 Rₐ groups;
R₆ and R₇ are each —H, or both R₆ and R₇ can be taken together to form a carbonyl;
R₈ is null, —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, aryl, arylalkyl, heteroaryl, heteroarylalkyl, 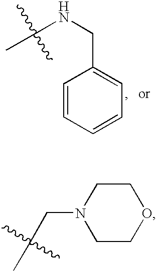
wherein all except H can be substituted with 0-5 Rₐ groups;
Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl; C₁₋₃ alkyl;
X₁, X₂, X₃, X₄, and X₅ are each independently —N—, —N⁺—, —C(R₁)—, or —C(H)—; [symbol]
denotes a single or double bond;
n is 0 or 1;
x is 0, 1, or 2; 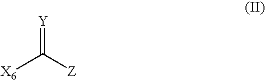
or a pharmaceutically acceptable salt, hydrate, or solvate thereof,
wherein
X₆ is NR₉R₁₀, or SR₁₁;
Y is NH, O, or S;
Z is NR₁₂R₁₃;
R₉, R₁₀, R₁₂, and R₁₃, are each independently —H, —OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, or C₃₋₆ cycloalkyl-C₁₋₃ alkyl, wherein all except H can be substituted with 0-5 Rₐ groups; or R₁₂ and R₁₃ can be taken together with the nitrogen to which they are attached to form a nitrogen containing 5- or 6-membered monocyclic heterocycle that can be substituted with 0-5 Rₐ groups;
R₁₁ is —H, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl, or 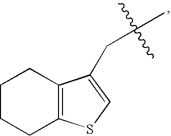
wherein all except H can be substituted with 0-5 Rₐ groups; or R₁₁ and R₁₂ can be taken together to form a 5- or 6-membered monocyclic heterocycle that can be substituted with 0-5 Rₐ groups;
Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl; NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl—C₁₋₃ alkyl, SH, SOₓC₁₋₃ alkyl, C₃₋₆ cycloalkyl, or SOₓC₃₋₆ cycloalkyl-C₁₋₃ alkyl;
x is 0, 1, or 2; 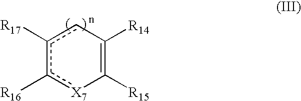
or a pharmaceutically acceptable salt, hydrate, or solvate thereof,
wherein
R₁₄, R₁₅, R₁₆ and R₁₇ are each independently —H, halogen, amino, hydroxyl, cyano, C₁₋₆ alkyl, C₂₋₆ alkenyl, C₂₋₆ alkynyl, C₁₋₆ alkoxy, C₃₋₆ cycloalkyl, C₃₋₆ cycloalkyl-C₁₋₃ alkyl, —C(O)OC₁₋₆ alkyl, —C(O)NHaryl, —C(O)NHC₁₋₆, alkyl, aryl, arylalkyl, heteroaryl, heteroarylalkyl, or 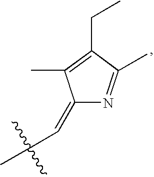
wherein all except H can be substituted with 0-5 Rₐ groups;
Rₐ is —H, halogen, CN, OH, alkylaryl, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆ cycloalkyl-C₁₋₃ alkyl; NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl;
X₇ is NH, S, O, or O⁺; [symbol]
denotes a single or double bond whereby no more than two of [symbol] can be a double bond;
n is 0 or 1; and
x is 0, 1, or 2;
whereby administration of one or more prodrug compounds of Formulae I, II, and III treats the infection by the pathogen.

14. The method of claim 13, wherein the pathogen is selected from the group consisting of a bacterium, a fungus, a protozoan, a helminth, and a combination thereof.

15. The method of claim 13, wherein the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacterpylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

16. The method of claim 13, wherein the infection is selected from the group consisting of an upper respiratory tract disease, an infection of a catheter, an infection of an orthopedic prostheses, a urinary tract infection, a gastrointestinal infection, a heart valve infection, endocarditis, a skin infection, a chronic wound, and cystic fibrosis.

17. The method of claim 13, wherein the prodrug compound is of Formula I.

18. The method of claim 13, wherein the prodrug compound is of Formula II.

19. The method of claim 13, wherein the prodrug compound is of Formula III.

20. A method of treating an infection by a pathogen in a patient in need thereof, the method comprising administering to the patient an effective amount of one or more analogs of prodrug Compounds 1-19: 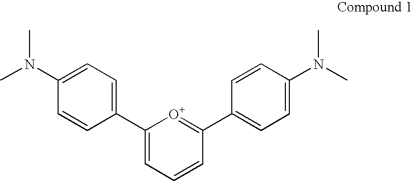
or a pharmaceutically acceptable salt, hydrate, or solvate thereof,
wherein any one or more of —H, and —N(CH₃)₂ can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 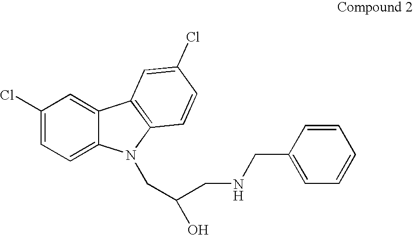
or a pharmaceutically acceptable salt, hydrate, or solvate thereof,
wherein any one or more of —H, —OH, —Cl, and —CH₂CH(OH)CH₂N(H)benzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆ alkyl; C₃₋₆ cycloalkyl; C₃₋₆ cycloalkyl-C₁₋₃ alkyl; alkylaryl; aryl; arylalkyl; heteroaryl; or heteroarylalkyl; 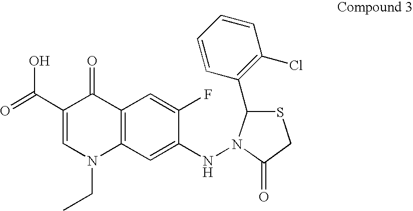
or a pharmaceutically acceptable salt, hydrate, or solvate thereof,
wherein any one or more of —H, —F, —CH2CH3,—C(O)OH, and chlorophenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆ alkyl; C₃₋₆ cycloalkyl; C₃₋₆ cycloalkyl-C₁₋₃ alkyl; alkylaryl; aryl; arylalkyl; heteroaryl; or heteroarylalkyl; 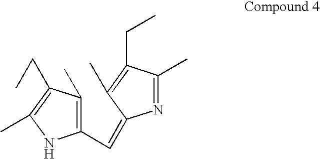
or a pharmaceutically acceptable salt, hydrate, or solvate thereof,
wherein any one or more of —H, —CH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the E configuration can be Z; 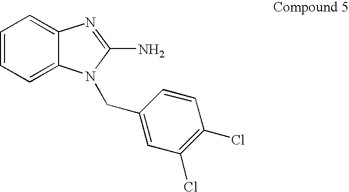
or a pharmaceutically acceptable salt, hydrate, or solvate thereof,
wherein any one or more of —H, —NH₂, and dichlorobenzyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 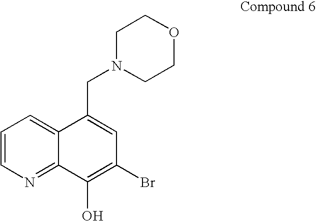
or a pharmaceutically acceptable salt, hydrate, or solvate thereof,
wherein any one or more of —H, —Br, —CH₂-morpholino, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 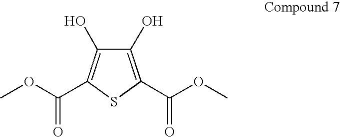
or a pharmaceutically acceptable salt, hydrate, or solvate thereof,
wherein any one or more of —H, —OH, —C(O)OCH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the thiophenyl —S— can be substituted with —O; 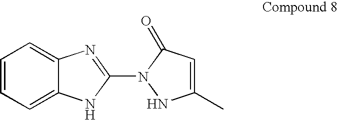
or a pharmaceutically acceptable salt, hydrate, or solvate thereof,
wherein any one or more of —H, and —CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 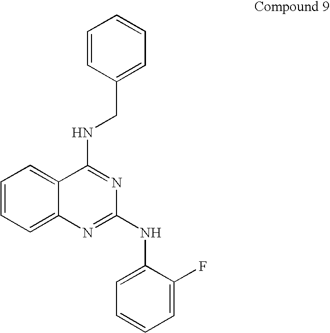
or a pharmaceutically acceptable salt, hydrate, or solvate thereof,
wherein any one or more of —H, —F, benzyl, and phenyl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 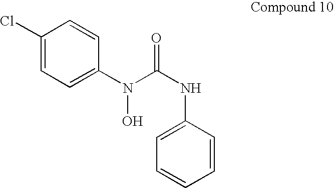
or a pharmaceutically acceptable salt, hydrate, or solvate thereof,
wherein any one or more of —H, —OH, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and —C(O) can be substituted with —C(S); 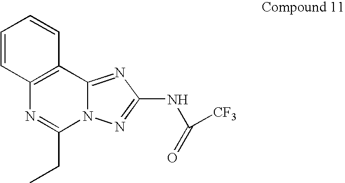
or a pharmaceutically acceptable salt, hydrate, or solvate thereof,
wherein any one or more of —H, —F, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 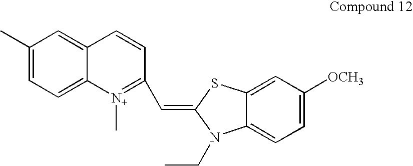
or a pharmaceutically acceptable salt, hydrate, or solvate thereof,
wherein any one or more of —H, —CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the Z configuration can be E; 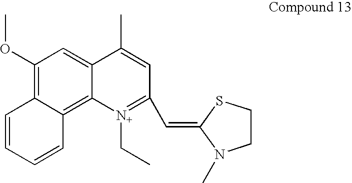
or a pharmaceutically acceptable salt, hydrate, or solvate thereof,
wherein any one or more of —H, CH₃, —OCH₃, —CH₂CH₃, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and the Z configuration can be E; 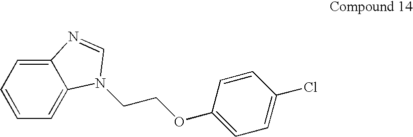
or a pharmaceutically acceptable salt, hydrate, or solvate thereof,
wherein any one or more of —H, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 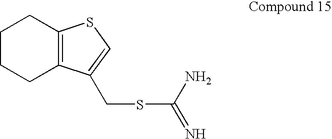
or a pharmaceutically acceptable salt, hydrate, or solvate thereof,
wherein any one or more of the following substituents, —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 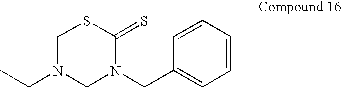
or a pharmaceutically acceptable salt, hydrate, or solvate thereof,
wherein any one or more of —H, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; 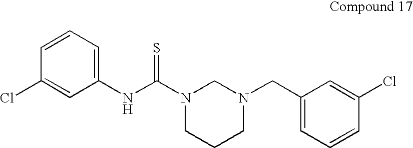
or a pharmaceutically acceptable salt, hydrate, or solvate thereof,
wherein any one or more of —H, —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; chlorophenyl can be substituted with heteroaryl, chlorobenzyl can be substituted with alkyl, alkylheteroaryl, and acyl; 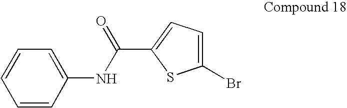
or a pharmaceutically acceptable salt, hydrate, or solvate thereof,
wherein any one or more of —H, and —Br, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; —C(O) can be substituted with —C(S), and S can be substituted with O; 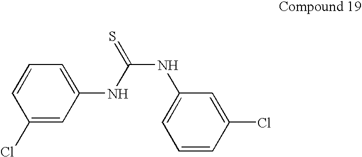
or a pharmaceutically acceptable salt, hydrate, or solvate thereof,
wherein any one or more of —H, and —Cl, can be substituted with any one of the following substituents: —H; halogen; —NO₂; —NH₂; hydroxyl; cyano; C₁₋₆ alkyl; C₂₋₆ alkenyl; C₂₋₆ alkynyl; C₁₋₆ alkoxy; —C(O)C₁₋₆alkyl; —C(O)OC₁₋₆alkyl; C₃₋₆ cycloalkyl; C₃₋₆ cycloalkyl-C₁₋₃ alkyl; alkylaryl; aryl; arylalkyl; heteroaryl; or heteroarylalkyl; and —C(S) can be substituted with —C(O);
whereby administration of one or more analogs of prodrug Compounds 1-19 treats the infection by the pathogen.

21. The method of claim 20, wherein the pathogen is selected from the group consisting of a bacterium, a fungus, a protozoan, a helminth, and a combination thereof.

22. The method of claim 20, wherein the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacterpylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

23. The method of claim 20, wherein the infection is selected from the group consisting of an upper respiratory tract disease, an infection of a catheter, an infection of an orthopedic prostheses, a urinary tract infection, a gastrointestinal infection, a heart valve infection, endocarditis, a skin infection, a chronic wound, and cystic fibrosis.

24. A method of treating an infection by a pathogen in a patient in need thereof, the method comprising administering to the patient an effective amount of one or more prodrug compounds of one or more of prodrug Compounds 1-19: 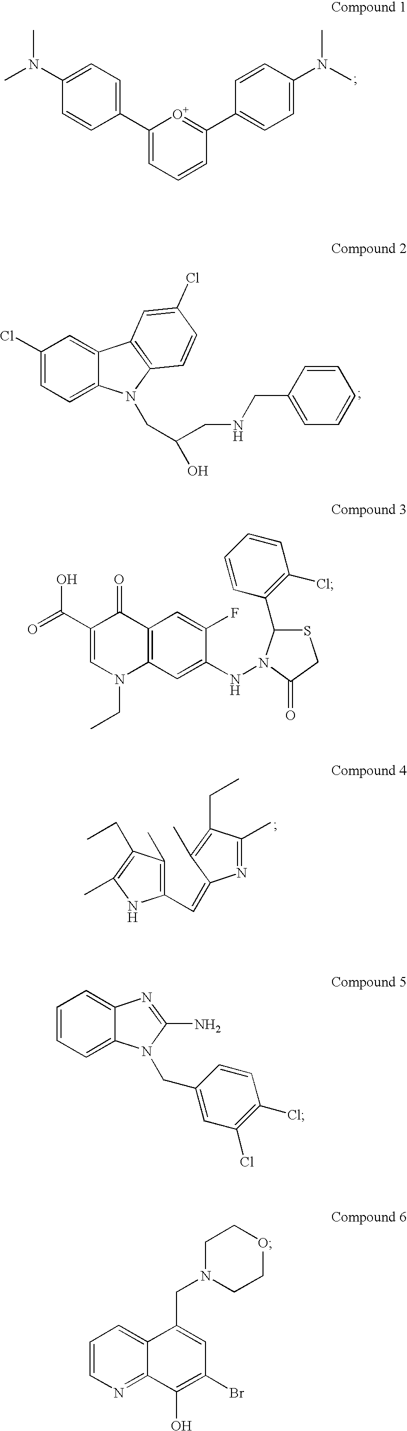 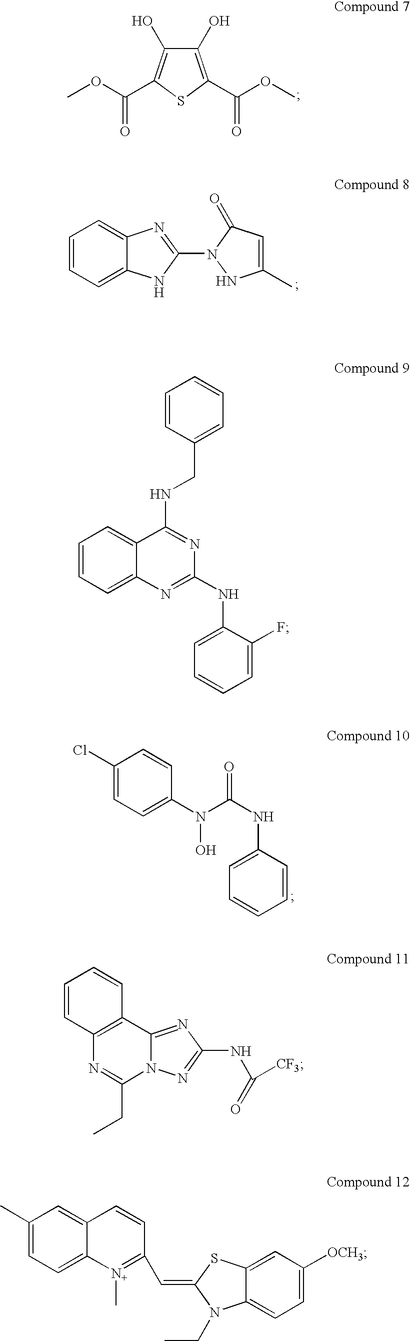 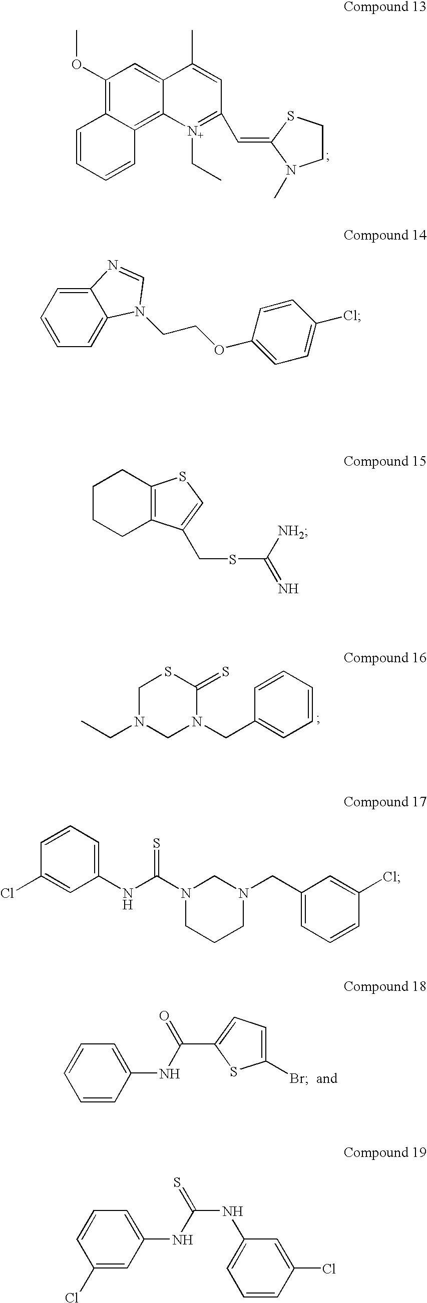
or a pharmaceutically acceptable salt, hydrate, or solvate of Compounds 1-19,
whereby administration of one or more of prodrug Compounds 1-19 treats the infection by the pathogen.

25. The method of claim 24, wherein the pathogen is selected from the group consisting of a bacterium, a fungus, a protozoan, a helminth, and a combination thereof.

26. The method of claim 24, wherein the pathogen is selected from the group consisting of Escherichia coli, Escherichia coli O157:H7, Escherichia coli UTI, Clostridium difficile, Campylobacter jejuni, Salmonella typhimurium, Staphylococcus aureus, Staphylococcus epidermidis, Listeria monocytogenes, Klebsiella pneumoniae, Haemophilus influenza, Helicobacter pylori, Pseudomonas aeruginosa, Burkholderia pseudomallei, Acinetobacter baumannii, Streptococcus pneumoniae, Streptococcus mutans, Enterococcus faecalis, Enterococcus faecium, Mycobacterium tuberculosis, Neisseria meningitidis, Bacillus anthracis, Bacillus brevis, Bacillus licheniformis, Bacillus megaterium, Bacillus pumilus, Bacillus subtilis, Bacillus vollum, Bacillus cepacia, Bacillus mallei, Bacillus thailandensis, Malleomyces mallei, Francisella tularensis, Yersinia pestis, Candida albicans, Candida glabrata, Aspergillus niger, Aspergillus fumigatus, Cryptococcus neoformans, Pneumocystis carinii, Plasmodium falciparum, Plasmodium vivax, Trypanosoma cruzeii, Entameoba histolytica, Entamoeba hartmanii, Dientamoeba fragilis, Giardia lamblia, Cryptosporidium parvum, Naegleria fowleri, Acanthomeaba SPP, Isospora belli, Microsporidia, flatworms, and roundworms.

27. The method of claim 24, wherein the infection is selected from the group consisting of an upper respiratory tract disease, an infection of a catheter, an infection of an orthopedic prostheses, a urinary tract infection, a gastrointestinal infection, a heart valve infection, endocarditis, a skin infection, a chronic wound, and cystic fibrosis.
